(19) United States
(12) Patent Application Publication
(10) Pub. No.: US 2023/0096380 A1
(43) Pub. Date: Mar. 30, 2023

(54) COMPOUNDS, COMPOSITIONS, METHODS FOR TREATING DISEASES AND NERVE DAMAGE, AND METHODS FOR PREPARING COMPOUNDS

(71) Applicant: CHILDREN'S HOSPITAL MEDICAL CENTER, Cincinnati, OH (US)

(72) Inventors: Qing LU, Cincinnati, OH (US); William SEIBEL, Liberty Township, OH (US)

(73) Assignee: CHILDREN'S HOSPITAL MEDICAL CENTER, Cincinnati, OH (US)

(21) Appl. No.: 17/759,724

(22) PCT Filed: Feb. 5, 2021
(86) PCT No.: PCT/US2021/016745

§ 371 Date: Jul. 29, 2022

Related U.S. Application Data
(60) Provisional application No. 62/970,765, filed on Feb. 6, 2020.

(51) Int. Cl. C07D 241/28; C07C 237/40; C07D 239/42; C07D 401/12; C07D 401/14; C07D 413/12; C07D 403/12; C07D 409/12

(52) U.S. Cl. CPC C07D 241/28; C07C 237/40; C07D 239/42; C07D 401/12; C07D 401/14; C07D 413/12; C07D 403/12; C07D 409/12

(57) ABSTRACT

Some embodiments of the invention include inventive compounds (e.g., compounds of Formula (I) or (Ia)). Other embodiments include compositions (e.g., pharmaceutical compositions) comprising the inventive compound. Still other embodiments of the invention include compositions (e.g., pharmaceutical compositions) for treating, for example, certain diseases or nerve injury using the inventive compounds. Some embodiments include methods of using the inventive compound (e.g., in compositions or in pharmaceutical compositions) for administering and treating (e.g., for treating disease, such as multiple sclerosis (MS), or for treating nerve damage). Further embodiments include methods for making the inventive compounds. Additional embodiments of the invention are also discussed herein.

### CROSS-REFERENCE TO RELATED APPLICATIONS

This application claims the benefit of U.S. Provisional Application No. 62/970,765, filed Feb. 6, 2020 entitled “COMPOUNDS, COMPOSITIONS, METHODS FOR TREATING DISEASES AND NERVE DAMAGE, AND METHODS FOR PREPARING COMPOUNDS” which is herein incorporated by reference in its entirety.

### BACKGROUND

Diseases (e.g., related to nerve function) and nerve injury are not uncommon. However, effective treatments for diseases or nerve injury remain a challenge. For example, current treatments for nerve injuries (e.g., spinal or PNS injury), such as by transection or crushing, are limited or ineffective. There is a need to develop effective treatments for nerve injuries. In another example, current treatments to treat (e.g., cure, halt, or slow) the progression of multiple sclerosis (MS) are limited or ineffective. MS is a prevalent demyelinating disease in the central nervous system (CNS) of both children and adults. There is a need for novel therapies to treat diseases (e.g., related to nerve function, such as MS) and nerve injury.

Certain embodiments of the invention address one or more of the deficiencies described above. Some embodiments of the invention include inventive compounds (e.g., compounds of Formula (I) or (Ia)). Other embodiments include compositions (e.g., pharmaceutical compositions) comprising the inventive compound. Still other embodiments of the invention include compositions (e.g., pharmaceutical compositions) for treating, for example, certain diseases or nerve injury using the inventive compounds. Some embodiments include methods of using the inventive compound (e.g., in compositions or in pharmaceutical compositions) for administering and treating (e.g., for treating disease, such as multiple sclerosis (MS), or for treating nerve damage). Further embodiments include methods for making the inventive compounds. Additional embodiments of the invention are also discussed herein.

### SUMMARY

Some embodiments of the present invention include a compound selected from Formula (I)

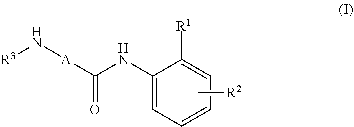

and salts, optical isomers, geometric isomers, salts of isomers, and derivatives thereof. In other embodiments, R¹ is —NH₂, hydroxy (—OH), —SH, —CN, methanoyl (—COH), or carboxy (—CO₂H). In still other embodiments, R² is monovalent H, halogen, hydroxy (—OH), methanoyl (—COH), carboxy (—CO₂H), nitro (—NO₂), —NH₂, —N(CH₃)₂, cyano (—CN), ethynyl (—CCH), propynyl, sulfo (—SO₃H), —CONH₂, —CON(CH₃)₂, C₁-C₃ alkyl, C₁-C₃ perfluorinated alkyl, —CF₃, —OCF₃, or C₁-C₃ alkoxy. In yet other embodiments, A is a cycloalkyl, heterocyclyl, aryl, or heteroaryl, which cycloalkyl, heterocyclyl, aryl, or heteroaryl is optionally substituted with one or more of halogen, oxo (═O), hydroxy (—OH), methanoyl (—COH), carboxy (—CO₂H), nitro (—NO₂), —NH₂, —N(CH₃)₂, cyano (—CN), ethynyl (—CCH), propynyl, sulfo (—SO₃H), morpholinyl, —CO-morpholin-4-yl, phenyl, —CONH₂, —CON(CH₃)₂, C₁-C₃ alkyl, C₁-C₃ perfluorinated alkyl, —CF₃, —OCF₃, or C₁-C₃ alkoxy. In certain embodiments, R³ can be methanoyl (—COH), carboxy (—CO₂H), C₁-C₁₀ alkyl, C₂-C₁₀ alkenyl, C₂-C₁₀ alkynyl, C₁-C₉ alkoxy, cycloalkyl, heterocyclyl, aryl, heteroaryl, or R^{Pa}, which methanoyl (—COH), carboxy (—CO₂H), C₁-C₁₀ alkyl, C₂-C₁₀ alkenyl, C₂-C₁₀ alkynyl, C₁-C₉ alkoxy, cycloalkyl, heterocyclyl, aryl, or heteroaryl is optionally substituted with one or more of halogen, oxo (═O), hydroxy (—OH), methanoyl (—COH), carboxy (—CO₂H), nitro (—NO₂), —NH₂, —N(CH₃)₂, cyano (—CN), ethynyl (—CCH), propynyl, sulfo (—SO₃H), morpholinyl, —CO-morpholin-4-yl, —O—CH₂-heteroaryl, —O—CH₂-heterocyclyl, —O—CH₂-phenyl, —O—CH₂-pyridinyl, —O—CH₂-pyrimidinyl, —O—CH₂-pyrazinyl, —CONH₂, —CON(CH₃)₂, C₁-C₅ alkyl, C₁-C₃ alkyl, methyl, ethyl, propyl, C₁-C₃ perfluorinated alkyl, —CF₃, —OCF₃, C₁-C₅ alkoxy, C₁-C₃ alkoxy, —O-phenyl, cycloalkyl, heterocyclyl, aryl, heteroaryl, pyridinyl, pyrimidinyl, pyrazinyl, —O(CO)H, —O(CO)(C₁-C₅ alkyl), —NH(CO)(C₁-C₅ alkyl), —N(C₁-C₅ alkyl)₂, —NH(C₁-C₅ alkyl), —O(CO)(C₁-C₃ alkyl), —NH(CO)(C₁-C₃ alkyl), —N(C₁-C₃ alkyl)₂, —NH(C₁-C₃ alkyl), —O(CO)(phenyl), —NH(CO)(phenyl), —N(C₁-C₃ alkyl)(phenyl), —NH(phenyl), phenyl, or R^{P}. In some embodiments, R^{P} is a phenyl substituted with one or more of halogen, hydroxy (—OH), methanoyl (—COH), carboxy (—CO₂H), nitro (—NO₂), —NH₂, —N(CH₃)₂, cyano (—CN), ethynyl (—CCH), propynyl, sulfo (—SO₃H), morpholinyl, —CO-morpholin-4-yl, —O—CH₂-heteroaryl, —O—CH₂-heterocyclyl, —O—CH₂-phenyl, —O—CH₂-pyridinyl, —O—CH₂-pyrimidinyl, —O—CH₂-pyrazinyl, —CONH₂, —CON(CH₃)₂, C₁-C₅ alkyl, C₁-C₃ alkyl, C₁-C₃ perfluorinated alkyl, —CF₃, —OCF₃, C₁-C₅ alkoxy, C₁-C₃ alkoxy, —O-phenyl, methyl, ethyl, propyl, cycloalkyl, heterocyclyl, aryl, heteroaryl, phenyl, pyridinyl, pyrimidinyl, pyrazinyl, —O(CO)H, —O(CO)(C₁-C₅ alkyl), —NH(CO)(C₁-C₅ alkyl), —N(C₁-C₅ alkyl)₂, —NH(C₁-C₅ alkyl), —O(CO)(C₁-C₃ alkyl), —NH(CO)(C₁-C₃ alkyl), —N(C₁-C₃ alkyl)₂, —NH(C₁-C₃ alkyl), —O(CO)(phenyl), —NH(CO)(phenyl), —N(C₁-C₃ alkyl)(phenyl), or —NH(phenyl). In other embodiments, R^{Pa} is a phenyl optionally substituted with one or more of halogen, hydroxy (—OH), methanoyl (—COH), carboxy (—CO₂H), nitro (—NO₂), —NH₂, —N(CH₃)₂, cyano (—CN), ethynyl (—CCH), propynyl, sulfo (—SO₃H), morpholinyl, —CO-morpholin-4-yl, —O—CH₂-heteroaryl, —O—CH₂-heterocyclyl, —O—CH₂-phenyl, —O—CH₂-pyridinyl, —O—CH₂-pyrimidinyl, —O—CH₂-pyrazinyl, —CONH₂, —CON(CH₃)₂, C₁-C₅ alkyl, C₁-C₃ alkyl, C₁-C₃ perfluorinated alkyl, —CF₃, —OCF₃, C₁-C₅ alkoxy, C₁-C₃ alkoxy, —O-phenyl, methyl, ethyl, propyl, cycloalkyl, heterocyclyl, aryl, heteroaryl, phenyl, pyridinyl, pyrimidinyl, pyrazinyl, —O(CO)H, —O(CO)(C₁-C₅ alkyl), —NH(CO)(C₁-C₅ alkyl), —N(C₁-C₅ alkyl)₂, —NH(C₁-C₅ alkyl), —O(CO)(C₁-C₃ alkyl), —NH(CO)(C₁-C₃ alkyl), —N(C₁-C₃ alkyl)₂, —NH(C₁-C₃ alkyl), —O(CO)(phenyl), —NH(CO)(phenyl), —N(C₁-C₃ alkyl)(phenyl), —NH(phenyl), or R^{P}. In certain embodiments, R^{P} and R^{Pa} can be the same or different.

In some embodiments, R¹ is —NH₂, R^{Pa} is a substituted phenyl, or both. In some embodiments, R¹ is —NH₂. In some embodiments, R^{Pa} is a substituted phenyl. In other embodiments, R² is (a) (i) meta to R¹ and para to the amide or (ii) para to R¹ and meta to the amide, (b) monovalent H, halogen, hydroxy (—OH), cyano (—CN), methyl, ethyl, or methoxy, or (c) both (a) and (b). In still other embodiments, A is a substituted or unsubstituted

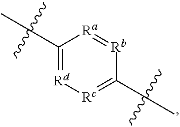

where R^{a}, R^{b}, R^{c}, and R^{d} is CH or N; R^{a}, R^{b}, R^{c}, and R^{d} can be the same or different from each other; the number of Ns in A is 0, 1, 2, or 3; and if A is substituted, it is substituted with one or more of halogen, hydroxy (—OH), methanoyl (—COH), carboxy (—CO₂H), nitro (—NO₂), —NH₂, —N(CH₃)₂, cyano (—CN), ethynyl (—CCH), propynyl, sulfo (—SO₃H), morpholinyl, —CO-morpholin-4-yl, phenyl, —CONH₂, —CON(CH₃)₂, C₁-C₃ alkyl, C₁-C₃ perfluorinated alkyl, —CF₃, —OCF₃, or C₁-C₃ alkoxy. In yet other embodiments, A is

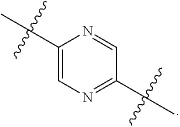 [SMILES: *c1cnc(*)cn1]

In still other embodiments, R^{P} is

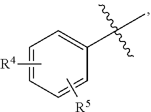

where R⁴ and R⁵ can be the same or different, can be ortho, para, or meta to each other, can each independently be ortho, para, or meta to the attachment point, and is H, halogen, hydroxy (—OH), methanoyl (—COH), carboxy (—CO₂H), nitro (—NO₂), —NH₂, —N(CH₃)₂, cyano (—CN), ethynyl (—CCH), propynyl, sulfo (—SO₃H), morpholinyl, —CO-morpholin-4-yl, —O—CH₂-heteroaryl, —O—CH₂-heterocyclyl, —O—CH₂-phenyl, —O—CH₂-pyridinyl, —O—CH₂-pyrimidinyl, —O—CH₂-pyrazinyl, —CONH₂, —CON(CH₃)₂, C₁-C₅ alkyl, C₁-C₃ alkyl, C₁-C₃ perfluorinated alkyl, —CF₃, —OCF₃, C₁-C₅ alkoxy, C₁-C₃ alkoxy, —O-phenyl, methyl, ethyl, propyl, cycloalkyl, heterocyclyl, aryl, heteroaryl, phenyl, pyridinyl, pyrimidinyl, pyrazinyl, —O(CO)H, —O(CO)(C₁-C₅ alkyl), —NH(CO)(C₁-C₅ alkyl), —N(C₁-C₅ alkyl)₂, —NH(C₁-C₅ alkyl), —O(CO)(C₁-C₃ alkyl), —NH(CO)(C₁-C₃ alkyl), —N(C₁-C₃ alkyl)₂, —NH(C₁-C₃ alkyl), —O(CO)(phenyl), —NH(CO)(phenyl), —N(C₁-C₃ alkyl)(phenyl), or —NH(phenyl). In yet other embodiments, R⁴ and R⁵ can be the same or different, can be ortho, para, or meta to each other, can each independently be ortho, para, or meta to the attachment point, and is H, F, Cl, Br, hydroxy (—OH), cyano (—CN), —CF₃, methyl, ethyl, methoxy, ethoxy, —O(CO)CH₃, or —NH—(CO)—CH₃.

In some embodiments, R^{Pa} is

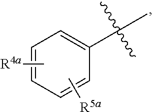

where R^{4a} and R^{5a} can be the same or different, can be ortho, para, or meta to each other, can each independently be ortho, para, or meta to the attachment point, and is H, halogen, hydroxy (—OH), methanoyl (—COH), carboxy (—CO₂H), nitro (—NO₂), —NH₂, —N(CH₃)₂, cyano (—CN), ethynyl (—CCH), propynyl, sulfo (—SO₃H), morpholinyl, —CO-morpholin-4-yl, —O—CH₂-heteroaryl, —O—CH₂-heterocyclyl, —O—CH₂-phenyl, —O—CH₂-pyridinyl, —O—CH₂-pyrimidinyl, —O—CH₂-pyrazinyl, —CONH₂, —CON(CH₃)₂, C₁-C₅ alkyl, C₁-C₃ alkyl, C₁-C₃ perfluorinated alkyl, —CF₃, —OCF₃, C₁-C₅ alkoxy, C₁-C₃ alkoxy, —O— phenyl, methyl, ethyl, propyl, cycloalkyl, heterocyclyl, aryl, heteroaryl, phenyl, pyridinyl, pyrimidinyl, pyrazinyl, —O(CO)H, —O(CO)(C₁-C₅ alkyl), —NH(CO)(C₁-C₅ alkyl), —N(C₁-C₅ alkyl)₂, —NH(C₁-C₅ alkyl), —O(CO)(C₁-C₃ alkyl), —NH(CO)(C₁-C₃ alkyl), —N(C₁-C₃ alkyl)₂, —NH(C₁-C₃ alkyl), —O(CO)(phenyl), —NH(CO)(phenyl), —N(C₁-C₃ alkyl)(phenyl), or —NH(phenyl). In certain embodiments, R^{4a} and R^{5a} can be the same or different, can be ortho, para, or meta to each other, can each independently be ortho, para, or meta to the attachment point, and is H, F, Cl, Br, hydroxy (—OH), cyano (—CN), —CF₃, methyl, ethyl, methoxy, ethoxy, —O(CO)CH₃, or —NH—(CO)—CH₃.

In other embodiments, R³ is

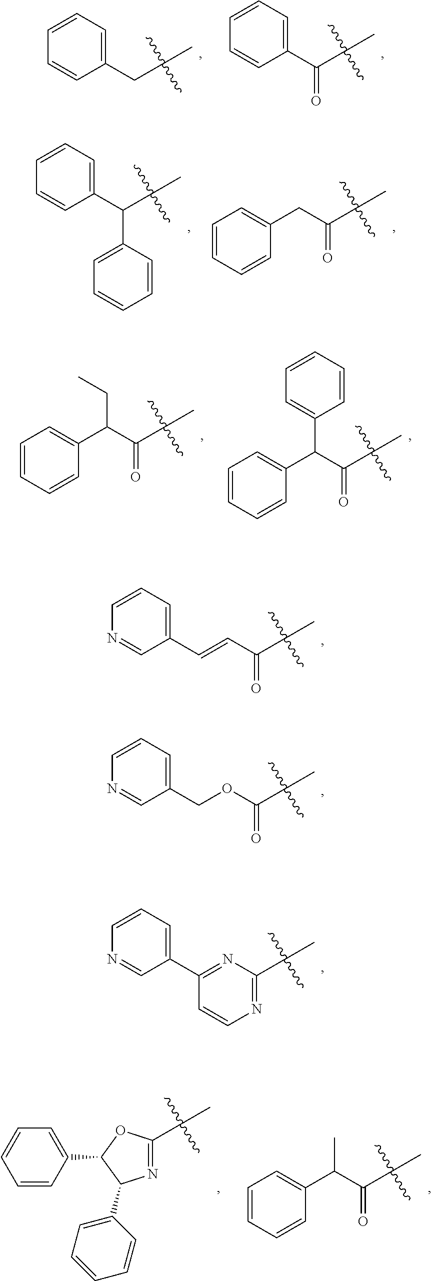

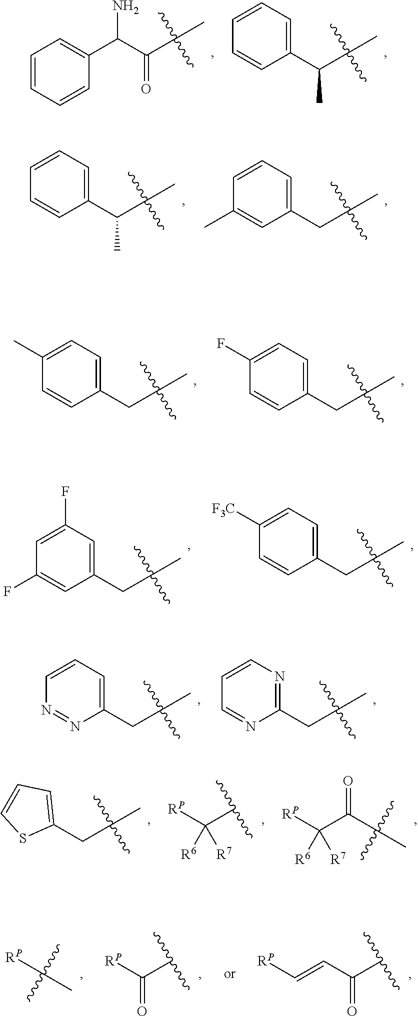

where R⁶ and R⁷ can be the same or different and is H, methyl, ethyl, phenyl, R^{P}, or pyridinyl.

In certain embodiments, R³ is

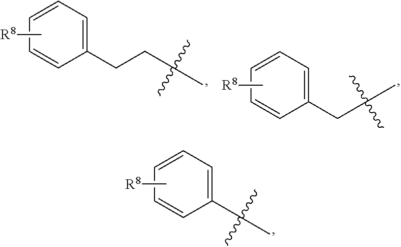

where R⁸ is ortho, para or meta to the attachment point or to the carbon in the phenyl associated with the attachment point, and is H, halogen (e.g., F, Cl, Br, or I), hydroxy (—OH), —CF₃, —CF₂CF₃, methanoyl (—COH), carboxy (—CO₂H), nitro (—NO₂), —NH₂, —N(CH₃)₂, cyano (—CN), sulfo (—SO₃H), —CONH₂, —CON(CH₃)₂, C₁-C₅ alkyl, C₁-C₃ alkyl, C₁-C₅ alkoxy, C₁-C₃ alkoxy, —O-phenyl, methyl, ethyl, propyl, phenyl, pyridinyl, pyrimidinyl, pyrazinyl, pyridazinyl, thienyl, —O(CO)H, —O(CO)(C₁-C₅ alkyl), —NH(CO)(C₁-C₅ alkyl), —N(C₁-C₅ alkyl)₂, —NH(C₁-C₅ alkyl), —O(CO)(C₁-C₃ alkyl), —NH(CO)(C₁-C₃ alkyl), —N(C₁-C₃ alkyl)₂, —NH(C₁-C₃ alkyl), —O(CO)(phenyl), —NH(CO)(phenyl), —N(C₁-C₃ alkyl)(phenyl), or —NH(phenyl).

In yet other embodiments, R³ does not comprise an oxo adjacent to the attachment point.

In some embodiments, the compound is selected from Formula (Ia)

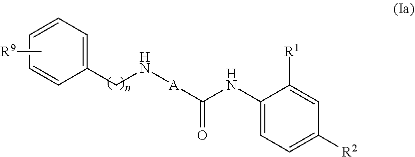

and salts, optical isomers, geometric isomers, salts of isomers, and derivatives thereof. In other embodiments, n is 0, 1, 2, or 3, and R⁹ can be ortho, para, or meta to the other connecting carbon on the phenyl, and is H, halogen, hydroxy (—OH), methanoyl (—COH), carboxy (—CO₂H), nitro (—NO₂), —NH₂, —N(CH₃)₂, cyano (—CN), ethynyl (—CCH), propynyl, sulfo (—SO₃H), morpholinyl, —CO-morpholin-4-yl, —O—CH₂-heteroaryl, —O—CH₂-heterocyclyl, —O—CH₂-phenyl, —O—CH₂-pyridinyl, —O—CH₂-pyrimidinyl, —O—CH₂-pyrazinyl, —CONH₂, —CON(CH₃)₂, C₁-C₅ alkyl, C₁-C₃ alkyl, C₁-C₃ perfluorinated alkyl, —CF₃, —OCF₃, C₁-C₅ alkoxy, C₁-C₃ alkoxy, —O-phenyl, methyl, ethyl, propyl, cycloalkyl, heterocyclyl, aryl, heteroaryl, phenyl, pyridinyl, pyrimidinyl, pyrazinyl, —O(CO)H, —O(CO)(C₁-C₅ alkyl), —NH(CO)(C₁-C₅ alkyl), —N(C₁-C₅ alkyl)₂, —NH(C₁-C₅ alkyl), —O(CO)(C₁-C₃ alkyl), —NH(CO)(C₁-C₃ alkyl), —N(C₁-C₃ alkyl)₂, —NH(C₁-C₃ alkyl), —O(CO)(phenyl), —NH(CO)(phenyl), —N(C₁-C₃ alkyl)(phenyl), or —NH(phenyl).

In certain embodiments, R⁹ can be ortho, para, or meta to the other connecting carbon on the phenyl, and is H, F, Cl, Br, hydroxy (—OH), cyano (—CN), methyl, ethyl, methoxy, ethoxy, —CF₃, —O(CO)CH₃, or —NH—(CO)—CH₃.

In other embodiments, the compound of Formula (I) is I-1, I-2, I-3, I-4, I-5, I-6, I-7, I-8, I-9, I-10, I-11, I-12, I-13, I-14, I-15, I-16, I-17, I-18, I-19, I-20, I-21, I-22, I-23, I-24, I-25, I-26, or I-27.

Some embodiments of the invention include a composition comprising a compound, as disclosed herein (e.g., Formula (I)). In certain embodiments, the amount of the compound is from about 0.0001% (by weight total composition) to about 99%. In other embodiments, the composition further comprises a formulary ingredient, an adjuvant, or a carrier.

Some embodiments of the invention include a pharmaceutical composition comprising a compound, as disclosed herein (e.g., Formula (I)). In some embodiments, the amount of the compound is from about 0.0001% (by weight total composition) to about 50%. In other embodiments, the pharmaceutical composition further comprises a formulary ingredient, an adjuvant, or a carrier.

Some embodiments of the invention include a method for providing an animal with a compound comprising one or more administrations of one or more compositions comprising a compound as disclosed herein (e.g., Formula (I)), wherein the compositions may be the same or different if there is more than one administration. In other embodiments, at least one of the one or more compositions further comprises a formulary ingredient. In still other embodiments, at least one of the one or more compositions comprises a composition (e.g., as disclosed herein) or a pharmaceutical composition (e.g., as disclosed herein). In certain embodiments, at least one of the one or more administrations comprises parenteral administration, a mucosal administration, intravenous administration, subcutaneous administration, topical administration, intradermal administration, oral administration, sublingual administration, intranasal administration, or intramuscular administration. In other embodiments, if there is more than one administration at least one composition used for at least one administration is different from the composition of at least one other administration. In still other embodiments, the compound of at least one of the one or more compositions is administered to the animal in an amount of from about 0.01 mg/kg animal body weight to about 15 mg/kg animal body weight. In yet other embodiments, the animal is a human, a rodent, or a primate.

Some embodiments of the invention include a method for treating an animal for a disease or a nerve injury, comprising one or more administrations of one or more compositions comprising a compound as disclosed herein (e.g., Formula (I)), wherein the compositions may be the same or different if there is more than one administration. In other embodiments, the composition further comprises a formulary ingredient. In still other embodiments, at least one of the one or more compositions comprises a composition (e.g., as disclosed herein) or a pharmaceutical composition (e.g., as disclosed herein). In certain embodiments, the composition is a pharmaceutical composition. In some embodiments, the administration comprises parenteral administration, mucosal administration, intravenous administration, depot injection, subcutaneous administration, topical administration, intradermal administration, oral administration, sublingual administration, intranasal administration, or intramuscular administration. In other embodiments, the administration comprises a depot injection or an oral administration. In still other embodiments, if there is more than one administration at least one composition used for at least one administration is different from the composition of at least one other administration. In yet other embodiments, the amount of the compound is from about 0.0001% (by weight total composition) to about 99%. In certain embodiments, the compound of the composition is administered to the animal in an amount of from about 0.005 mg/kg animal body weight to about 100 mg/kg animal body weight. In some embodiments, the animal is a human, a rodent, or a primate. In other embodiments, the animal is in need of treatment of a disease or a nerve injury. In still other embodiments, the method is for treating myelopathy, spinal cord injury, myelitis, vascular myelopathy, cervical spondylotic myelopathy, spondylosis, spinal stenosis, demyelinating disease, any disease of the nervous system where the myelin sheath of a neuron is damaged, CNS demyelinating disease, PNS demyelinating disease, genetic demyelinating disease, infectious demyelinating disease, autoimmune demyelinating disease, demyelinating myelinoclastic disease, demyelinating leukodystrophic disease, Devic's disease, CNS neuropathies, diseases resulting in vitamin B12 deficiency, central pontine myelinolysis, myelopathies, tabes dorsalis, leukoencephalopathies, progressive multifocal leukoencephalopathy, leukodystrophies, optic neuritis, transverse myelitis, neuromyelitis optica, Guillain-Barré syndrome, chronic inflammatory demyelinating polyneuropathy, anti-MAG peripheral neuropathy, Charcot-Marie-Tooth disease, Hereditary neuropathy with liability to pressure palsy, copper deficiency associated conditions, peripheral neuropathy, myelopathy, optic neuropathy, progressive inflammatory neuropathy, multiple sclerosis (MS), MS-type clinically isolated syndrome, relapsing-remitting MS, primary progressive MS, secondary progressive MS, Alzheimer's Disease, amyotrophic lateral sclerosis (ALS), and Huntington's Disease, traumatic brain injury, acquired brain injury, hypoxic ischemic brain injury, strokes, periventricular leukomalacia (PVL), white-matter brain injury, CNS nerve injury, PNS nerve injury, crush nerve injury, or transection nerve injury. In some embodiments, the method is for treating MS, MS-type clinically isolated syndrome, relapsing-remitting MS, primary progressive MS, or secondary progressive MS. In certain embodiments, the method is for treating inflammation, remyelination, or both in MS, MS-type clinically isolated syndrome, relapsing-remitting MS, primary progressive MS, or secondary progressive MS. In other embodiments, the method is for treating inflammation and remyelination in MS, MS-type clinically isolated syndrome, relapsing-remitting MS, primary progressive MS, or secondary progressive MS. In still other embodiments, the method is for treating CNS demyelinating disease, PNS demyelinating disease, MS, Alzheimer's Disease, amyotrophic lateral sclerosis (ALS), and Huntington's Disease, traumatic brain injury, acquired brain injury, hypoxic ischemic brain injury, strokes, periventricular leukomalacia (PVL), white-matter brain injury, CNS nerve injury, PNS nerve injury, crush nerve injury, or transection nerve injury. In yet other embodiments, the method is for treating CNS demyelinating disease, PNS demyelinating disease, MS, Alzheimer's Disease, amyotrophic lateral sclerosis (ALS), and Huntington's Disease, CNS nerve injury, PNS nerve injury, crush nerve injury, or transection nerve injury. In certain embodiments, the method is for treating CNS nerve injury, PNS nerve injury, crush nerve injury, or transection nerve injury. In some embodiments, the method further comprises one or more other treatments.

Some embodiments of the invention include a method for treating an animal for MS or nerve injury, comprising administration to the animal of a composition comprising a compound selected from Formula (Ia) and salts, optical isomers, geometric isomers, salts of isomers, and derivatives thereof.

Some embodiments of the invention include a method for treating an animal for MS or nerve injury, comprising administration to the animal of a composition comprising a compound selected from compound I-1 and salts, optical isomers, geometric isomers, salts of isomers, and derivatives thereof.

Some embodiments of the invention include a method for preparing a compound as disclosed herein (e.g., Formula (I)) comprising, (a) reacting a compound of Formula (II) with a compound of Formula (III) to result in a mixture comprising a compound of Formula (IV), (b) reacting a compound of Formula (IV) to result in a mixture comprising a compound of Formula (V), (c) reacting a compound of Formula (V) with a compound of Formula (VI), and (d) recovering a compound of Formula (I). In other embodiments, Formula (II) is

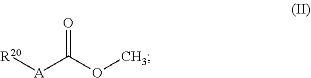

### Formula (III) is

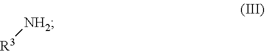

### Formula (IV) is

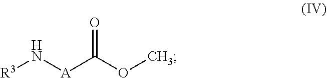

### Formula (V) is

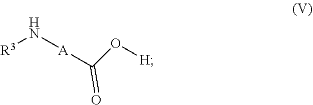

### Formula (VI) is

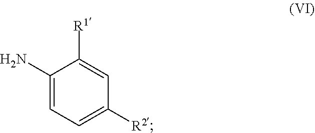

R²⁰ is a halogen; R^{1′} is R¹ or R¹ with a protecting group; and R^{2′} is R² or R² with a protecting group. In other embodiments, the compound is selected from Formula (Ia). In still other embodiments, in (b) Formula (IV) is reacted with a base. In yet other embodiments, in (b) Formula (IV) is reacted with a base, and the base is LiOH. In certain embodiments, one or both of R^{1′} or R^{2′} is a protected R¹ or a protected R². In other embodiments, (i) one or both of R^{1′} or R^{2′} is a protected R¹ or a protected R² and (ii) one or both protecting groups are a carbamate, t-butoxycarbonyl (Boc), benzyloxycarbonyl (Cbz), or 9-fluorenylmethoxycarbonyl (Fmoc). In some embodiments, (i) one or both of R^{1′} or R^{2′} is a protected R¹ or a protected R² and (ii) after (c), the protecting group(s) are removed from one or both of R^{1′} or R^{2′}.

Other embodiments of the invention are also discussed herein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following drawings form part of the present specification and are included to further demonstrate certain aspects of the present invention. The invention may be better understood by reference to one or more of these drawings in combination with the description of specific embodiments presented herein.

FIG. 1: Compound I-1 promotes oligodendrocyte progenitor cell (OPC) differentiation.

FIG. 2: A485 (a histone acetyltransferase inhibitor; CAS #1889279-16-6) inhibits p300 activity. Yet, compound I-1 appears to overcome A485 inhibition to enhance myelin formation.

FIG. 3: Compound I-1 promotes OPC differentiation.

FIG. 4: Compound I-1 promotes OPC differentiation.

FIG. 5: Compound I-1 enhances myelin MBP+ cell formation.

FIG. 6: Compound I-1 enhances myelin MBP+ cell formation 3 days after treatment.

FIG. 7: Treatment with compound I-1 promotes functional regeneration and reduced EAE disease severity in mice.

FIG. 8: Mouse bodyweight analysis after treatment with compound I-1 and RGFP966.

FIG. 9: Organ coefficient to brain weight after treatment with compound I-1 and RGFP966.

FIG. 10: Mouse food consumption after treatment with compound I-1 and RGFP966.

FIG. 11: Biochemistry examination analysis after treatment with compound I-1 and RGFP966.

FIG. 12: Hematological examination analysis after treatment with compound I-1 and RGFP966.

FIG. 13: Hemagglutination examination analysis after treatment with compound I-1 and RGFP966.

FIG. 14: Urine examination analysis after treatment with compound I-1 and RGFP966.

FIG. 15: Urine examination analysis after treatment with compound I-1 and RGFP966.

FIG. 16: Organ absolute weight after treatment with compound I-1 and RGFP966.

FIG. 17: Example data for EAE-VEP study.

FIG. 18: Compounds I-1, I-16, I-15, I-13, I-8, and I-17 promote OPC differentiation. Immunostaining for MBP (green) and Olig2 (red) in rat OPCs treated with DMSO, at the indicated concentration of the compounds.

### DETAILED DESCRIPTION

While embodiments encompassing the general inventive concepts may take diverse forms, various embodiments will be described herein, with the understanding that the present disclosure is to be considered merely exemplary, and the general inventive concepts are not intended to be limited to the disclosed embodiments.

Some embodiments of the invention include inventive compounds (e.g., compounds of Formula (I) or (Ia)). Other embodiments include compositions (e.g., pharmaceutical compositions) comprising the inventive compound. Still other embodiments of the invention include compositions (e.g., pharmaceutical compositions) for treating, for example, certain diseases or nerve injury using the inventive compounds. Some embodiments include methods of using the inventive compound (e.g., in compositions or in pharmaceutical compositions) for administering and treating (e.g., for treating disease, such as multiple sclerosis (MS), or for treating nerve damage). Further embodiments include methods for making the inventive compounds. Additional embodiments of the invention are also discussed herein.

As used herein (unless otherwise specified), the term “alkyl” means a monovalent, straight or branched hydrocarbon chain. For example, the terms “C₁-C₇ alkyl” or “C₁-C₄ alkyl” refer to straight- or branched-chain saturated hydrocarbon groups having from 1 to 7 (e.g., 1, 2, 3, 4, 5, 6, or 7), or 1 to 4 (e.g., 1, 2, 3, or 4), carbon atoms, respectively. Examples of C₁-C₇ alkyl groups include, but are not limited to, methyl, ethyl, n-propyl, i-propyl, n-butyl, s-butyl, t-butyl, n-pentyl, s-pentyl, n-hexyl, and n-septyl. Examples of C₁-C₄ alkyl groups include, but are not limited to, methyl, ethyl, n-propyl, i-propyl, n-butyl, s-butyl, and t-butyl.

As used herein (unless otherwise specified), the term “alkenyl” means a monovalent, straight or branched hydrocarbon chain that includes one or more (e.g., 1, 2, 3, or 4) double bonds. Examples of alkenyl groups include, but are not limited to, vinyl, allyl, 1-propenyl, 2-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, and 5-hexenyl.

As used herein (unless otherwise specified), the term “alkoxy” means any of the above alkyl groups which is attached to the remainder of the molecule by an oxygen atom (alkyl-O—). Examples of alkoxy groups include, but are not limited to, methoxy (sometimes shown as MeO—), ethoxy, isopropoxy, propoxy, and butyloxy.

As used herein (unless otherwise specified), the term “alkynyl” means a monovalent, straight or branched hydrocarbon chain that includes one or more (e.g., 1, 2, 3, or 4) triple bonds and that also may optionally include one or more (e.g. 1, 2, 3, or 4) double bonds in the chain. Examples of alkynyl groups include, but are not limited to, ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, and 5-hexynyl.

As used herein (unless otherwise specified), the term “aryl” means a monovalent, monocyclic or bicyclic, 5, 6, 7, 8, 9, 10, 11, or 12 membered aromatic hydrocarbon group which, when unsubstituted. Examples of aryl groups include, but are not limited to, phenyl, naphthyl, tolyl, and xylyl. For a bicyclic aryl that is designated as substituted, one or both rings can be substituted.

As used herein (unless otherwise specified), the term “cycloalkyl” means a monovalent, monocyclic or bicyclic, 3, 4, 5, 6, 7, 8, 9, 10, 11, or 12 membered hydrocarbon group. The rings can be saturated or partially unsaturated. Examples of cycloalkyl groups include, but are not limited to, cyclopropyl, cyclobutyl, cyclopentyl, cyclohexyl, cycloheptyl, cyclooctyl, and bicycloalkyls (e.g., bicyclooctanes such as [2.2.2]bicyclooctane or [3.3.0]bicyclooctane, bicyclononanes such as [4.3.0]bicyclononane, and bicyclodecanes such as [4.4.0]bicyclodecane (decalin), or spiro compounds). For a monocyclic cycloalkyl, the ring is not aromatic. For a bicyclic cycloalkyl, if one ring is aromatic, then the other is not aromatic. For a bicyclic cycloalkyl that is designated as substituted, one or both rings can be substituted.

As used herein (unless otherwise specified), the term “halogen” means monovalent Cl, F, Br, or I.

As used herein (unless otherwise specified), the term “heteroaryl” means a monovalent, monocyclic or bicyclic, 5, 6, 7, 8, 9, 10, 11, or 12 membered, hydrocarbon group, where 1, 2, 3, 4, 5, or 6 carbon atoms are replaced by a hetero atom independently selected from nitrogen, oxygen, or sulfur atom, and the monocyclic or bicyclic ring system is aromatic. Examples of heteroaryl groups include, but are not limited to, thienyl (or thiophenyl), furyl, indolyl, pyrrolyl, pyridinyl, pyrazinyl, pyridazinyl, oxazolyl, thiaxolyl, quinolinyl, pyrimidinyl, imidazolyl, triazolyl, tetrazolyl, 1H-pyrazol-4-yl, 1-Me-pyrazol-4-yl, pyridin-3-yl, pyridin-4-yl, 3,5-dimethylisoxazolyl, 1H-pyrrol-3-yl, 3,5-di-Me-pyrazolyl, and 1H-pyrazol-4-yl. For a bicyclic heteroaryl, if one ring is aryl, then the other is heteroaryl. For a bicyclic heteroaryl, one or both rings can have one or more hetero atoms. For a bicyclic heteroaryl that is designated as substituted, one or both rings can be substituted.

As used herein (unless otherwise specified), the term “heterocyclyl” means a monovalent, monocyclic or bicyclic, 5, 6, 7, 8, 9, 10, 11, or 12 membered, hydrocarbon, where 1, 2, 3, 4, 5, or 6 carbon atoms are replaced by a hetero atom independently selected from nitrogen atom, oxygen atom, or sulfur atom, and the monocyclic or bicyclic ring system is not aromatic. Examples of heterocyclyl groups include, but are not limited to, tetrahydropyran, pyrolidinyl (e.g., pyrrolidin-1-yl, pyrrolidin-2-yl, pyrrolidin-3-yl, or pyrrolidin-4-yl), piperazinyl (e.g., piperazin-1-yl, piperazin-2-yl, piperazin-3-yl, or piperazin-4-yl), piperidinyl (e.g., piperadin-1-yl, piperadin-2-yl, piperadin-3-yl, or piperadin-4-yl), and morpholinyl (e.g., morpholin-1-yl, morpholin-2-yl, morpholin-3-yl, or morpholin-4-yl). For a bicyclic heterocyclyl, if one ring is aromatic (e.g., monocyclic aryl or heteroaryl), then the other ring is not aromatic. For a bicyclic heterocyclyl, one or both rings can have one or more hetero atoms. For a bicyclic heterocyclyl that is designated as substituted, one or both rings can be substituted.

As used herein (unless otherwise specified), the term “hetero atom” means an atom selected from nitrogen atom, oxygen atom, or sulfur atom.

As used herein (unless otherwise specified), the terms “hydroxy” or “hydroxyl” indicates the presence of a monovalent —OH group.

As used herein (unless otherwise specified), the term “substituted” (e.g., as in substituted alkyl) means that one or more hydrogen atoms of a chemical group (with one or more hydrogen atoms) can be replaced by one or more non-hydrogen substituents selected from the specified options. The replacement can occur at one or more positions. The term “optionally substituted” means that one or more hydrogen atoms of a chemical group (with one or more hydrogen atoms) can be, but is not required to be, substituted.

Some compounds of the invention can have one or more chiral centers and can exist in and be isolated in optically active and racemic forms, for any of the one or more chiral centers. Some compounds can exhibit polymorphism. The compounds of the present invention (e.g., Formula I) encompass any optically active, racemate, stereoisomer form, polymorphism, or mixtures thereof. If a chiral center does not provide an indication of its configuration (i.e., R or S) in a chemical structure, it should be considered to represent R, S or a racemate.

Compounds and Compositions Including Pharmaceutical Compositions

Some embodiments of the invention include compounds of Formula (I):

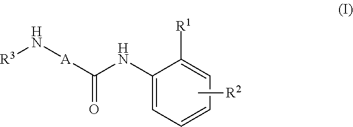

(I) and salts, optical isomers, geometric isomers, salts of isomers, and derivatives thereof.

In some embodiments, R¹ can be —NH₂, hydroxy (—OH), —SH, —CN, methanoyl (—COH), or carboxy (—CO₂H). In other embodiments, R¹ can be —NH₂, hydroxy (—OH), or —SH. In still other embodiments, R¹ can be —NH₂ or hydroxy (—OH). In yet other embodiments, R¹ can be —NH₂.

In some embodiments, R² can be ortho, meta, or para to R₁, can be ortho, meta, or para to the attached amide (i.e., connecting to A), and can be monovalent H, halogen (e.g., F, Cl, Br, or I), hydroxy (—OH), methanoyl (—COH), carboxy (—CO₂H), nitro (—NO₂), —NH₂, —N(CH₃)₂, cyano (—CN), ethynyl (—CCH), propynyl, sulfo (—SO₃H), —CONH₂, —CON(CH₃)₂, C₁-C₃ alkyl, C₁-C₃ perfluorinated alkyl, —CF₃, —OCF₃, or C₁-C₃ alkoxy. In other embodiments, R² can be monovalent H, halogen (e.g., F, Cl, Br, or I), hydroxy (—OH), methanoyl (—COH), carboxy (—CO₂H), nitro (—NO₂), —NH₂, cyano (—CN), ethynyl (—CCH), propynyl, C₁-C₃ alkyl, C₁-C₃ perfluorinated alkyl, —CF₃, —OCF₃, or C₁-C₃ alkoxy. In other embodiments, R² can be monovalent H, halogen (e.g., F, Cl, Br, or I), hydroxy (—OH), methanoyl (—COH), carboxy (—CO₂H), nitro (—NO₂), —NH₂, cyano (—CN), ethynyl (—CCH), propynyl, C₁-C₂ alkyl, C₁-C₂ perfluorinated alkyl, —CF₃, —OCF₃, or C₁-C₂ alkoxy. In other embodiments, R² can be monovalent H, halogen (e.g., F, Cl, Br, or I), hydroxy (—OH), methanoyl (—COH), cyano (—CN), ethynyl (—CCH), C₁-C₂ alkyl, C₁-C₂ perfluorinated alkyl, —CF₃, —OCF₃, or methoxy. In certain embodiments, R² can be monovalent H, halogen (e.g., F, Cl, Br, or I), hydroxy (—OH), cyano (—CN), methyl, ethyl, or methoxy. In some embodiments, R² can be positioned as

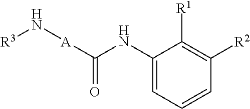

(ortho to R¹ and meta to the amide),

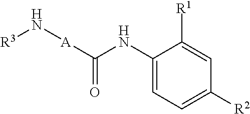

(meta to R¹ and para to the amide),

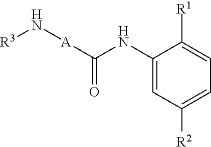

(para to R¹ and meta to the amide), or

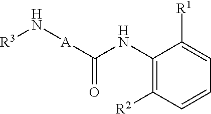

(meta to R¹ and ortho to the amide). In other embodiments, R² can be positioned as

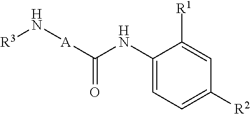

(meta to R¹ and para to the amide).

In some embodiments, A can be cycloalkyl (e.g., a 4-, 5-, 6-, or 7-membered cycloalkyl), heterocyclyl (e.g., a 5-, 6-, or 7-membered heterocyclyl), aryl (e.g., a 5-, 6-, or 7-membered aryl), or heteroaryl (e.g., a 5-, 6-, or 7-membered heteroaryl), which cycloalkyl (e.g., a 4-, 5-, 6-, or 7-membered cycloalkyl), heterocyclyl (e.g., a 5-, 6-, or 7-membered heterocyclyl), aryl (e.g., a 5-, 6-, or 7-membered aryl), or heteroaryl (e.g., a 5-, 6-, or 7-membered heteroaryl) can optionally be substituted with one or more (e.g., 0, 1, 2, 3, 4, 5, or 6) of halogen (e.g., F, Cl, Br, or I), oxo (═O), hydroxy (—OH), methanoyl (—COH), carboxy (—CO₂H), nitro (—NO₂), —NH₂, —N(CH₃)₂, cyano (—CN), ethynyl (—CCH), propynyl, sulfo (—SO₃H), morpholinyl, —CO-morpholin-4-yl, phenyl, —CONH₂, —CON(CH₃)₂, C₁-C₃ alkyl, C₁-C₃ perfluorinated alkyl, —CF₃, —OCF₃, or C₁-C₃ alkoxy. In other embodiments, A is a 5-membered cycloalkyl, heterocyclyl, aryl, or heteroaryl with 0, 1, or 2 nitrogens in the ring. In other embodiments, A is a 6-membered cycloalkyl, heterocyclyl, aryl, or heteroaryl with 0, 1, 2, or 3 nitrogens in the ring. In some embodiments, A is a substituted (e.g., substituted with one or more (e.g., 0, 1, 2, 3, 4, 5, or 6) of halogen (e.g., F, Cl, Br, or I), oxo (═O), hydroxy (—OH), methanoyl (—COH), carboxy (—CO₂H), nitro (—NO₂), —NH₂, —N(CH₃)₂, cyano (—CN), ethynyl (—CCH), propynyl, sulfo (—SO₃H), morpholinyl, —CO-morpholin-4-yl, phenyl, —CONH₂, —CON(CH₃)₂, C₁-C₃ alkyl, C₁-C₃ perfluorinated alkyl, —CF₃, —OCF₃, or C₁-C₃ alkoxy) or unsubstituted

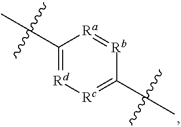

where R^{a}, R^{b}, R^{c}, and R^{d}, can be the same or different from each other and can be CH or N, where the number of Ns in A is 0, 1, 2, or 3. In other embodiments, R^{a} is N, R^{c} is N, R^{b} is CH, and R^{d} is CH. In yet other embodiments, R^{a} is CH, R^{c} is CH, R^{b} is N, and R^{d} is N. In other embodiments, R^{a} is N, R^{c} is CH, R^{b} is N, and R^{d} is CH. In other embodiments, R^{a} is CH, R^{c} is N, R^{b} is CH, and R^{d} is N. In other embodiments, R^{a} is CH, R^{c} is N, R^{b} is N, and R^{d} is CH. In other embodiments, R^{a} is N, R^{c} is CH, R^{b} is CH, and R^{d} is N. In some embodiments, only one of R^{a}, R^{b}, R^{c}, or R^{d} is an N, and the others are CH. In some embodiments, only one of R^{a}, R^{b}, R^{c}, or R^{d} is a CH, and the others are N. In some embodiments, A is a substituted (e.g., substituted with one or more (e.g., 0, 1, 2, 3, 4, 5, or 6) of halogen (e.g., F, Cl, Br, or I), oxo (═O), hydroxy (—OH), methanoyl (—COH), carboxy (—CO₂H), nitro (—NO₂), —NH₂, —N(CH₃)₂, cyano (—CN), ethynyl (—CCH), propynyl, sulfo (—SO₃H), morpholinyl, —CO-morpholin-4-yl, phenyl, —CONH₂, —CON(CH₃)₂, C₁-C₃ alkyl, C₁-C₃ perfluorinated alkyl, —CF₃, —OCF₃, or C₁-C₃ alkoxy) or unsubstituted phenyl, pyridinyl, pyrimidinyl, or pyrazinyl. In some embodiments, A is an unsubstituted phenyl or pyrazinyl. In certain embodiments, A is

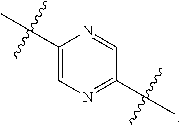 [SMILES: *c1cnc(*)cn1]

In some embodiments, R³ can be methanoyl (—COH), carboxy (—CO₂H), C₁-C₁₀ alkyl (e.g., C₁, C₂, C₃, C₄, C₅, C₆, C₇, C₈, C₉, or C₁₀ alkyl), C₂-C₁₀ alkenyl (e.g., C₂, C₃, C₄, C₅, C₆, C₇, C₈, C₉, or C₁₀ alkenyl), C₂-C₁₀ alkynyl (e.g., C₂, C₃, C₄, C₅, C₆, C₇, C₈, C₉, or C₁₀ alkynyl), C₁-C₉ alkoxy (e.g., C₁, C₂, C₃, C₄, C₅, C₆, C₇, C₈, or C₉ alkoxy), cycloalkyl, heterocyclyl, aryl, heteroaryl, or R^{Pa}, which methanoyl (—COH), carboxy (—CO₂H), C₁-C₁₀ alkyl, C₂-C₁₀ alkenyl, C₂-C₁₀ alkynyl, C₁-C₉ alkoxy, cycloalkyl, heterocyclyl, aryl, heteroaryl, or R^{Pa} can optionally be substituted with one or more (e.g., 0, 1, 2, 3, 4, 5, or 6) of halogen (e.g., F, Cl, Br, or I), oxo (═O), hydroxy (—OH), methanoyl (—COH), carboxy (—CO₂H), nitro (—NO₂), —NH₂, —N(CH₃)₂, cyano (—CN), ethynyl (—CCH), propynyl, sulfo (—SO₃H), morpholinyl, —CO-morpholin-4-yl, —O—CH₂-heteroaryl, —O—CH₂-heterocyclyl, —O—CH₂-phenyl, —O—CH₂-pyridinyl, —O—CH₂-pyrimidinyl, —O—CH₂-pyrazinyl, —CONH₂, —CON(CH₃)₂, C₁-C₅ alkyl, C₁-C₃ alkyl, methyl, ethyl, propyl, C₁-C₃ perfluorinated alkyl, —CF₃, —OCF₃, C₁-C₅ alkoxy, C₁-C₃ alkoxy, —O-phenyl, cycloalkyl, heterocyclyl, aryl, heteroaryl, pyridinyl, pyrimidinyl, pyrazinyl, —O(CO)H, —O(CO)(C₁-C₅ alkyl), —NH(CO)(C₁-C₅ alkyl), —N(C₁-C₅ alkyl)₂, —NH(C₁-C₅ alkyl), —O(CO)(C₁-C₃ alkyl), —NH(CO)(C₁-C₃ alkyl), —N(C₁-C₃ alkyl)₂, —NH(C₁-C₃ alkyl), —O(CO)(phenyl), —NH(CO)(phenyl), —N(C₁-C₃ alkyl)(phenyl), —NH(phenyl), phenyl, or R^{P} (which can be the same or different from R^{Pa}). In some embodiments, R^{P} or R^{Pa} (which can be the same or different) is a substituted phenyl (e.g., a phenyl substituted with one or more (e.g., 1, 2, 3, 4, or 5) of halogen (e.g., F, Cl, Br, or I), hydroxy (—OH), methanoyl (—COH), carboxy (—CO₂H), nitro (—NO₂), —NH₂, —N(CH₃)₂, cyano (—CN), ethynyl (—CCH), propynyl, sulfo (—SO₃H), morpholinyl, —CO-morpholin-4-yl, —O—CH₂-heteroaryl, —O—CH₂-heterocyclyl, —O—CH₂-phenyl, —O—CH₂-pyridinyl, —O—CH₂-pyrimidinyl, —O—CH₂-pyrazinyl, —CONH₂, —CON(CH₃)₂, C₁-C₅ alkyl, C₁-C₃ alkyl, C₁-C₃ perfluorinated alkyl, —CF₃, —OCF₃, C₁-C₅ alkoxy, C₁-C₃ alkoxy, —O-phenyl, methyl, ethyl, propyl, cycloalkyl, heterocyclyl, aryl, heteroaryl, phenyl, pyridinyl, pyrimidinyl, pyrazinyl, —O(CO)H, —O(CO)(C₁-C₅ alkyl), —NH(CO)(C₁-C₅ alkyl), —N(C₁-C₅ alkyl)₂, —NH(C₁-C₅ alkyl), —O(CO)(C₁-C₃ alkyl), —NH(CO)(C₁-C₃ alkyl), —N(C₁-C₃ alkyl)₂, —NH(C₁-C₃ alkyl), —O(CO)(phenyl), —NH(CO)(phenyl), —N(C₁-C₃ alkyl)(phenyl), or —NH(phenyl)). In some embodiments, R^{Pa} (which can be the same or different) is a substituted phenyl (e.g., a phenyl substituted with one or more (e.g., 1, 2, 3, 4, or 5) of halogen (e.g., F, Cl, Br, or I), hydroxy (—OH), methanoyl (—COH), carboxy (—CO₂H), nitro (—NO₂), —NH₂, —N(CH₃)₂, cyano (—CN), ethynyl (—CCH), propynyl, sulfo (—SO₃H), morpholinyl, —CO-morpholin-4-yl, —O—CH₂-heteroaryl, —O—CH₂-heterocyclyl, —O—CH₂-phenyl, —O—CH₂-pyridinyl, —O—CH₂-pyrimidinyl, —O—CH₂-pyrazinyl, —CONH₂, —CON(CH₃)₂, C₁-C₅ alkyl, C₁-C₃ alkyl, C₁-C₃ perfluorinated alkyl, —CF₃, —OCF₃, C₁-C₅ alkoxy, C₁-C₃ alkoxy, —O-phenyl, methyl, ethyl, propyl, cycloalkyl, heterocyclyl, aryl, heteroaryl, phenyl, pyridinyl, pyrimidinyl, pyrazinyl, —O(CO)H, —O(CO)(C₁-C₅ alkyl), —NH(CO)(C₁-C₅ alkyl), —N(C₁-C₅ alkyl)₂, —NH(C₁-C₅ alkyl), —O(CO)(C₁-C₃ alkyl), —NH(CO)(C₁-C₃ alkyl), —N(C₁-C₃ alkyl)₂, —NH(C₁-C₃ alkyl), —O(CO)(phenyl), —NH(CO)(phenyl), —N(C₁-C₃ alkyl)(phenyl), —NH(phenyl)), or R^{P}.

In other embodiments, R³ can be C₁-C₆ alkyl (e.g., C₁, C₂, C₃, C₄, C₅, or C₆ alkyl), C₂-C₆ alkenyl (e.g., C₂, C₃, C₄, C₅, or C₆ alkenyl), C₂-C₆ alkynyl (e.g., C₂, C₃, C₄, C₅, or C₆ alkynyl), C₁-C₅ alkoxy (e.g., C₁, C₂, C₃, C₄, or C₅ alkoxy), cycloalkyl, heterocyclyl, aryl, heteroaryl, or R^{Pa}, which C₁-C₆ alkyl, C₂-C₆ alkenyl, C₂-C₆ alkynyl, C₁-C₅ alkoxy, cycloalkyl, heterocyclyl, aryl, heteroaryl, or R^{Pa} (e.g., R^{Pa} is an optionally substituted phenyl) can optionally be substituted with one or more (e.g., 0, 1, 2, 3, 4, 5, or 6) of halogen (e.g., F, Cl, Br, or I), oxo (═O), hydroxy (—OH), methanoyl (—COH), carboxy (—CO₂H), nitro (—NO₂), —NH₂, —N(CH₃)₂, cyano (—CN), sulfo (—SO₃H), —O—CH₂-heteroaryl, —O—CH₂-heterocyclyl, —O—CH₂-phenyl, —O—CH₂-pyridinyl, —O—CH₂-pyrimidinyl, —O—CH₂-pyrazinyl, —CONH₂, —CON(CH₃)₂, C₁-C₅ alkyl, C₁-C₃ alkyl, methyl, ethyl, propyl, C₁-C₃ perfluorinated alkyl, —CF₃, —OCF₃, C₁-C₅ alkoxy, C₁-C₃ alkoxy, —O-phenyl, heterocyclyl, aryl, heteroaryl, pyridinyl, pyrimidinyl, pyrazinyl, —O(CO)H, —O(CO)(C₁-C₅ alkyl), —NH(CO)(C₁-C₅ alkyl), —N(C₁-C₅ alkyl)₂, —NH(C₁-C₅ alkyl), —O(CO)(C₁-C₃ alkyl), —NH(CO)(C₁-C₃ alkyl), —N(C₁-C₃ alkyl)₂, —NH(C₁-C₃ alkyl), —O(CO)(phenyl), —NH(CO)(phenyl), —N(C₁-C₃ alkyl)(phenyl), —NH(phenyl), phenyl, or R^{P} (which can be the same or different from R^{Pa}). In some embodiments, R^{P} or R^{Pa} (which can be the same or different) is a optionally substituted phenyl (e.g., a phenyl substituted with one or more (e.g., 1, 2, 3, 4, or 5) of halogen (e.g., F, Cl, Br, or I), hydroxy (—OH), methanoyl (—COH), carboxy (—CO₂H), nitro (—NO₂), —NH₂, —N(CH₃)₂, cyano (—CN), sulfo (—SO₃H), —CONH₂, —CON(CH₃)₂, C₁-C₅ alkyl, C₁-C₃ alkyl, C₁-C₃ perfluorinated alkyl, —CF₃, —OCF₃, C₁-C₅ alkoxy, C₁-C₃ alkoxy, —O-phenyl, methyl, ethyl, propyl, heterocyclyl, aryl, heteroaryl, phenyl, pyridinyl, pyrimidinyl, pyrazinyl, pyridazinyl, thienyl, —O(CO)H, —O(CO)(C₁-C₅ alkyl), —NH(CO)(C₁-C₅ alkyl), —N(C₁-C₅ alkyl)₂, —NH(C₁-C₅ alkyl), —O(CO)(C₁-C₃ alkyl), —NH(CO)(C₁-C₃ alkyl), —N(C₁-C₃ alkyl)₂, —NH(C₁-C₃ alkyl), —O(CO)(phenyl), —NH(CO)(phenyl), —N(C₁-C₃ alkyl)(phenyl), or —NH(phenyl)).

In some embodiments, R^{P} is

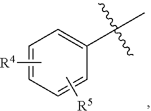

where R⁴ and R⁵ can be the same or different, can be ortho, para, or meta to each other, can each independently be ortho, para, or meta to the attachment point, and can be H, halogen (e.g., F, Cl, Br, or I), hydroxy (—OH), methanoyl (—COH), carboxy (—CO₂H), nitro (—NO₂), —NH₂, —N(CH₃)₂, cyano (—CN), ethynyl (—CCH), propynyl, sulfo (—SO₃H), morpholinyl, —CO-morpholin-4-yl, —O—CH₂-heteroaryl, —O—CH₂-heterocyclyl, —O—CH₂-phenyl, —O—CH₂-pyridinyl, —O—CH₂-pyrimidinyl, —O—CH₂-pyrazinyl, —CONH₂, —CON(CH₃)₂, C₁-C₅ alkyl, C₁-C₃ alkyl, C₁-C₃ perfluorinated alkyl, —CF₃, —OCF₃, C₁-C₅ alkoxy, C₁-C₃ alkoxy, —O-phenyl, methyl, ethyl, propyl, cycloalkyl, heterocyclyl, aryl, heteroaryl, phenyl, pyridinyl, pyrimidinyl, pyrazinyl, —O(CO)H, —O(CO)(C₁-C₅ alkyl), —NH(CO)(C₁-C₅ alkyl), —N(C₁-C₅ alkyl)₂, —NH(C₁-C₅ alkyl), —O(CO)(C₁-C₃ alkyl), —NH(CO)(C₁-C₃ alkyl), —N(C₁-C₃ alkyl)₂, —NH(C₁-C₃ alkyl), —O(CO)(phenyl), —NH(CO)(phenyl), —N(C₁-C₃ alkyl)(phenyl), or —NH(phenyl). In other embodiments, R⁴ and R⁵ can be the same or different, can be ortho, para, or meta to each other, can each independently be ortho, para, or meta to the attachment point, and can be H, halogen (e.g., F, Cl, Br, or I), hydroxy (—OH), methanoyl (—COH), carboxy (—CO₂H), nitro (—NO₂), —NH₂, —N(CH₃)₂, cyano (—CN), sulfo (—SO₃H), —CONH₂, —CON(CH₃)₂, C₁-C₅ alkyl, C₁-C₃ alkyl, C₁-C₃ perfluorinated alkyl, —CF₃, —OCF₃, C₁-C₅ alkoxy, C₁-C₃alkoxy, —O-phenyl, methyl, ethyl, propyl, cycloalkyl, heterocyclyl, aryl, heteroaryl, phenyl, pyridinyl, pyrimidinyl, pyrazinyl, —O(CO)H, —O(CO)(C₁-C₅ alkyl), —NH(CO)(C₁-C₅ alkyl), —N(C₁-C₅ alkyl)₂, —NH(C₁-C₅ alkyl), —O(CO)(C₁-C₃ alkyl), —NH(CO)(C₁-C₃ alkyl), —N(C₁-C₃ alkyl)₂, —NH(C₁-C₃ alkyl), —O(CO)(phenyl), —NH(CO)(phenyl), —N(C₁-C₃ alkyl)(phenyl), or —NH(phenyl). In yet other embodiments, R⁴ and R⁵ can be the same or different, can be ortho, para, or meta to each other, can each independently be ortho, para, or meta to the attachment point, and can be H, halogen (e.g., F, Cl, Br, or I), hydroxy (—OH), methanoyl (—COH), carboxy (—CO₂H), nitro (—NO₂), —NH₂, —N(CH₃)₂, cyano (—CN), sulfo (—SO₃H), —CONH₂, —CON(CH₃)₂, C₁-C₅ alkyl, C₁-C₃ alkyl, C₁-C₅ alkoxy, C₁-C₃ alkoxy, —O— phenyl, methyl, ethyl, propyl, phenyl, pyridinyl, pyrimidinyl, pyrazinyl, —O(CO)H, —O(CO)(C₁-C₅ alkyl), —NH(CO)(C₁-C₅ alkyl), —N(C₁-C₅ alkyl)₂, —NH(C₁-C₅ alkyl), —O(CO)(C₁-C₃ alkyl), —NH(CO)(C₁-C₃ alkyl), —N(C₁-C₃ alkyl)₂, —NH(C₁-C₃ alkyl), —O(CO)(phenyl), —NH(CO)(phenyl), —N(C₁-C₃ alkyl)(phenyl), or —NH(phenyl). In still other embodiments, R⁴ and R⁵ can be the same or different, can be ortho, para, or meta to each other, can each independently be ortho, para, or meta to the attachment point, and can be H, halogen (e.g., F, Cl, Br, or I), hydroxy (—OH), cyano (—CN), C₁-C₅ alkyl, C₁-C₃ alkyl, C₁-C₅ alkoxy, C₁-C₃ alkoxy, methyl, ethyl, propyl, methoxy, ethoxy, —O(CO)H, —O(CO)(C₁-C₅ alkyl), —NH(CO)(C₁-C₅ alkyl), —O(CO)(C₁-C₃ alkyl), —NH(CO)(C₁-C₃ alkyl), —O(CO)CH₃, or —NH—(CO)—CH₃. In yet other embodiments, R⁴ and R⁵ can be the same or different, can be ortho, para, or meta to each other, can each independently be ortho, para, or meta to the attachment point, and can be H, F, C₁, Br, hydroxy (—OH), cyano (—CN), methyl, ethyl, methoxy, ethoxy, —O(CO)CH₃, or —NH—(CO)—CH₃. In some embodiments, R⁴ can be ortho, para, or meta to the attachment point. In certain embodiments, R⁵ can be ortho, para, or meta to the attachment point.

In some embodiments, R^{Pa} is

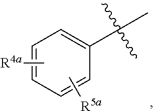

where R^{4a} and R^{5a} can be the same or different, can be ortho, para, or meta to each other, can each independently be ortho, para, or meta to the attachment point, and can be H, halogen (e.g., F, Cl, Br, or I), hydroxy (—OH), methanoyl (—COH), carboxy (—CO₂H), nitro (—NO₂), —NH₂, —N(CH₃)₂, cyano (—CN), ethynyl (—CCH), propynyl, sulfo (—SO₃H), morpholinyl, —CO-morpholin-4-yl, —O—CH₂-heteroaryl, —O—CH₂-heterocyclyl, —O—CH₂-phenyl, —O—CH₂-pyridinyl, —O—CH₂-pyrimidinyl, —O—CH₂-pyrazinyl, —CONH₂, —CON(CH₃)₂, C₁-C₅ alkyl, C₁-C₃ alkyl, C₁-C₃ perfluorinated alkyl, —CF₃, —OCF₃, C₁-C₅ alkoxy, C₁-C₃ alkoxy, —O-phenyl, methyl, ethyl, propyl, cycloalkyl, heterocyclyl, aryl, heteroaryl, phenyl, pyridinyl, pyrimidinyl, pyrazinyl, —O(CO)H, —O(CO)(C₁-C₅ alkyl), —NH(CO)(C₁-C₅ alkyl), —N(C₁-C₅ alkyl)₂, —NH(C₁-C₅ alkyl), —O(CO)(C₁-C₃ alkyl), —NH(CO)(C₁-C₃ alkyl), —N(C₁-C₃ alkyl)₂, —NH(C₁-C₃ alkyl), —O(CO)(phenyl), —NH(CO)(phenyl), —N(C₁-C₃ alkyl)(phenyl), or —NH(phenyl). In other embodiments, R^{4a} and R^{5a} can be the same or different, can be ortho, para, or meta to each other, can each independently be ortho, para, or meta to the attachment point, and can be H, halogen (e.g., F, Cl, Br, or I), hydroxy (—OH), methanoyl (—COH), carboxy (—CO₂H), nitro (—NO₂), —NH₂, —N(CH₃)₂, cyano (—CN), sulfo (—SO₃H), —CONH₂, —CON(CH₃)₂, C₁-C₅ alkyl, C₁-C₃ alkyl, C₁-C₃ perfluorinated alkyl, —CF₃, —OCF₃, C₁-C₅ alkoxy, C₁-C₃alkoxy, —O-phenyl, methyl, ethyl, propyl, cycloalkyl, heterocyclyl, aryl, heteroaryl, phenyl, pyridinyl, pyrimidinyl, pyrazinyl, —O(CO)H, —O(CO)(C₁-C₅ alkyl), —NH(CO)(C₁-C₅ alkyl), —N(C₁-C₅ alkyl)₂, —NH(C₁-C₅ alkyl), —O(CO)(C₁-C₃ alkyl), —NH(CO)(C₁-C₃ alkyl), —N(C₁-C₃ alkyl)₂, —NH(C₁-C₃ alkyl), —O(CO)(phenyl), —NH(CO)(phenyl), —N(C₁-C₃ alkyl)(phenyl), or —NH(phenyl). In yet other embodiments, R^{4a} and R^{5a} can be the same or different, can be ortho, para, or meta to each other, can each independently be ortho, para, or meta to the attachment point, and can be H, halogen (e.g., F, Cl, Br, or I), hydroxy (—OH), methanoyl (—COH), carboxy (—CO₂H), nitro (—NO₂), —NH₂, —N(CH₃)₂, cyano (—CN), sulfo (—SO₃H), —CONH₂, —CON(CH₃)₂, C₁-C₅ alkyl, C₁-C₃ alkyl, C₁-C₅ alkoxy, C₁-C₃ alkoxy, —O— phenyl, methyl, ethyl, propyl, phenyl, pyridinyl, pyrimidinyl, pyrazinyl, —O(CO)H, —O(CO)(C₁-C₅ alkyl), —NH(CO)(C₁-C₅ alkyl), —N(C₁-C₅ alkyl)₂, —NH(C₁-C₅ alkyl), —O(CO)(C₁-C₃ alkyl), —NH(CO)(C₁-C₃ alkyl), —N(C₁-C₃ alkyl)₂, —NH(C₁-C₃ alkyl), —O(CO)(phenyl), —NH(CO)(phenyl), —N(C₁-C₃ alkyl)(phenyl), or —NH(phenyl). In still other embodiments, R^{4a} and R^{5a} can be the same or different, can be ortho, para, or meta to each other, can each independently be ortho, para, or meta to the attachment point, and can be H, halogen (e.g., F, Cl, Br, or I), hydroxy (—OH), cyano (—CN), C₁-C₅ alkyl, C₁-C₃ alkyl, C₁-C₅ alkoxy, C₁-C₃ alkoxy, methyl, ethyl, propyl, methoxy, ethoxy, —O(CO)H, —O(CO)(C₁-C₅ alkyl), —NH(CO)(C₁-C₅ alkyl), —O(CO)(C₁-C₃ alkyl), —NH(CO)(C₁-C₃ alkyl), —O(CO)CH₃, or —NH—(CO)—CH₃. In yet other embodiments, R^{4a} and R^{5a} can be the same or different, can be ortho, para, or meta to each other, can each independently be ortho, para, or meta to the attachment point, and can be H, F, Cl, Br, hydroxy (—OH), cyano (—CN), methyl, ethyl, methoxy, ethoxy, —O(CO)CH₃, or —NH—(CO)—CH₃. In some embodiments, R^{4a} can be ortho, para, or meta to the attachment point. In certain embodiments, R^{5a} can be ortho, para, or meta to the attachment point.

In certain embodiments, R³ is

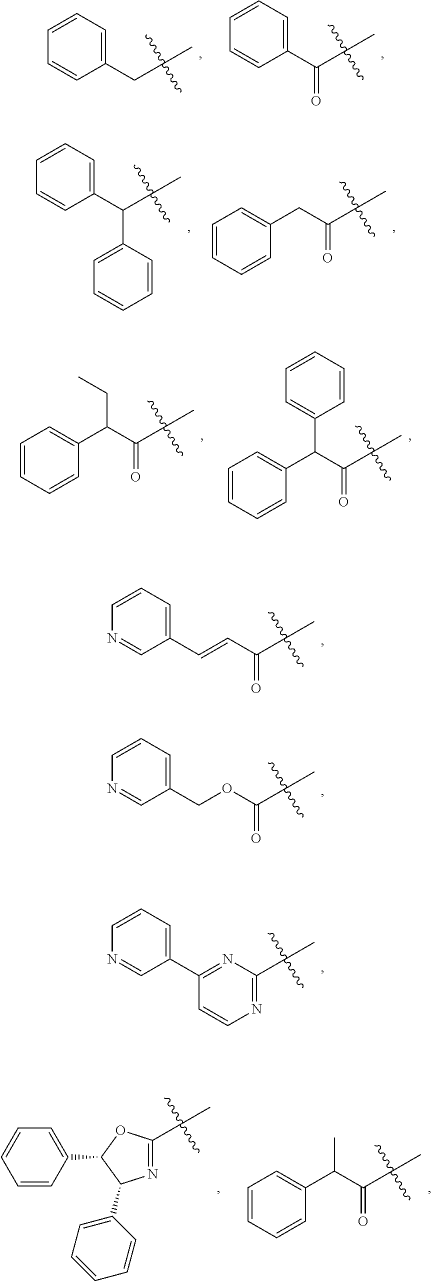

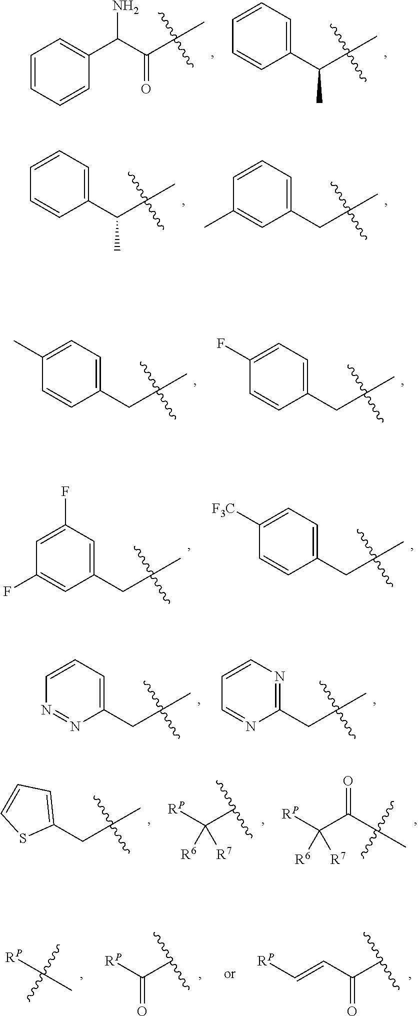

where R⁶ and R⁷ can be the same or different and can be H, methyl, ethyl, phenyl, R^{P}, or pyridinyl.

In some embodiments, R³ is

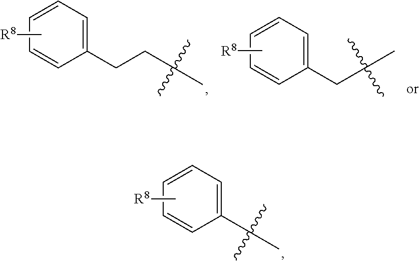

where R⁸ can be ortho, para or meta to the attachment point or to the carbon in the phenyl associated with the attachment point, and can be H, halogen (e.g., F, Cl, Br, or I), hydroxy (—OH), —CF₃, —CF₂CF₃, methanoyl (—COH), carboxy (—CO₂H), nitro (—NO₂), —NH₂, —N(CH₃)₂, cyano (—CN), sulfo (—SO₃H), —CONH₂, —CON(CH₃)₂, C₁-C₅ alkyl, C₁-C₃ alkyl, C₁-C₅ alkoxy, C₁-C₃ alkoxy, —O-phenyl, methyl, ethyl, propyl, phenyl, pyridinyl, pyrimidinyl, pyrazinyl, pyridazinyl, thienyl, —O(CO)H, —O(CO)(C₁-C₅ alkyl), —NH(CO)(C₁-C₅ alkyl), —N(C₁-C₅ alkyl)₂, —NH(C₁-C₅ alkyl), —O(CO)(C₁-C₃ alkyl), —NH(CO)(C₁-C₃ alkyl), —N(C₁-C₃ alkyl)₂, —NH(C₁-C₃ alkyl), —O(CO)(phenyl), —NH(CO)(phenyl), —N(C₁-C₃ alkyl)(phenyl), or —NH(phenyl).

In other embodiments, R³ is no

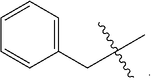 [SMILES: *Cc1ccccc1]

In some embodiments, R³ does not comprise an oxo (i.e., —(CO)—) adjacent to the attachment point (i.e., adjacent to —NH—{ . . . }, to which R³ is attached); that is, there is no

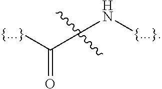

In other embodiments, R³ does comprise an oxo (i.e., —(CO)—) adjacent to the attachment point (i.e., adjacent to —NH—{ . . . }, to which R³ is attached); that is, there is

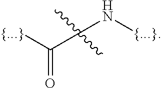

In some embodiments, Formula (I) is Formula (Ia):

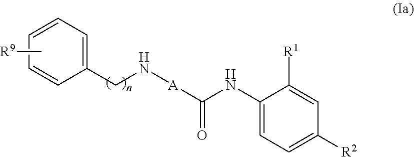

and salts, optical isomers, geometric isomers, salts of isomers, and derivatives thereof.

In some embodiments, R¹ can be any R¹ disclosed herein. In certain embodiments, R¹ can be —NH₂, hydroxy (—OH), —SH, —CN, methanoyl (—COH), or carboxy (—CO₂H). In other embodiments, R¹ can be —NH₂, hydroxy (—OH), or —SH. In still other embodiments, R¹ can be —NH₂ or hydroxy (—OH). In yet other embodiments, R¹ can be —NH₂.

In some embodiments, R² can be any R² disclosed herein. In certain embodiments, R² can be monovalent H, halogen (e.g., F, Cl, Br, or I), hydroxy (—OH), methanoyl (—COH), carboxy (—CO₂H), nitro (—NO₂), —NH₂, —N(CH₃)₂, cyano (—CN), ethynyl (—CCH), propynyl, sulfo (—SO₃H), —CONH₂, —CON(CH₃)₂, C₁-C₃ alkyl, C₁-C₃ perfluorinated alkyl, —CF₃, —OCF₃, or C₁-C₃ alkoxy. In other embodiments, R² can be monovalent H, halogen (e.g., F, Cl, Br, or I), hydroxy (—OH), methanoyl (—COH), carboxy (—CO₂H), nitro (—NO₂), —NH₂, cyano (—CN), ethynyl (—CCH), propynyl, C₁-C₃ alkyl, C₁-C₃ perfluorinated alkyl, —CF₃, —OCF₃, or C₁-C₃ alkoxy. In other embodiments, R² can be monovalent H, halogen (e.g., F, Cl, Br, or I), hydroxy (—OH), methanoyl (—COH), carboxy (—CO₂H), nitro (—NO₂), —NH₂, cyano (—CN), ethynyl (—CCH), propynyl, C₁-C₂ alkyl, C₁-C₂ perfluorinated alkyl, —CF₃, —OCF₃, or C₁-C₂ alkoxy. In other embodiments, R² can be monovalent H, halogen (e.g., F, Cl, Br, or I), hydroxy (—OH), methanoyl (—COH), cyano (—CN), ethynyl (—CCH), C₁-C₂ alkyl, C₁-C₂ perfluorinated alkyl, —CF₃, —OCF₃, or methoxy. In certain embodiments, R² can be monovalent H, halogen (e.g., F, Cl, Br, or I), hydroxy (—OH), cyano (—CN), methyl, ethyl, or methoxy.

In some embodiments, A can be any A disclosed herein. In other embodiments, A can be a substituted (e.g., substituted with one or more (e.g., 0, 1, 2, 3, 4, 5, or 6) of halogen (e.g., F, Cl, Br, or I), oxo (═O), hydroxy (—OH), methanoyl (—COH), carboxy (—CO₂H), nitro (—NO₂), —NH₂, —N(CH₃)₂, cyano (—CN), ethynyl (—CCH), propynyl, sulfo (—SO₃H), morpholinyl, —CO-morpholin-4-yl, phenyl, —CONH₂, —CON(CH₃)₂, C₁-C₃ alkyl, C₁-C₃ perfluorinated alkyl, —CF₃, —OCF₃, or C₁-C₃ alkoxy) or unsubstituted

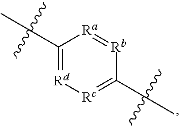

where R^{a}, R^{b}, R^{c}, and R^{d}, can be the same or different from each other and can be CH or N, where the number of Ns in A is 0, 1, 2, or 3. In other embodiments, R^{a} is N, R^{c} is N, R^{b} is CH, and R^{d} is CH. In yet other embodiments, R^{a} is CH, R^{c} is CH, R^{b} is N, and R^{d} is N. In other embodiments, R^{a} is N, R^{c} is CH, R^{b} is N, and R^{d} is CH. In other embodiments, R^{a} is CH, R^{c} is N, R^{b} is CH, and R^{d} is N. In other embodiments, R^{a} is CH, R^{c} is N, R^{b} is N, and R^{d} is CH. In other embodiments, R^{a} is N, R^{c} is CH, R^{b} is CH, and R^{d} is N. In some embodiments, only one of R^{a}, R^{b}, R^{c}, or R^{d} is an N, and the others are CH. In some embodiments, only one of R^{a}, R^{b}, R^{c}, or R^{d} is a CH, and the others are N. In some embodiments, A is a substituted (e.g., substituted with one or more (e.g., 0, 1, 2, 3, 4, 5, or 6) of halogen (e.g., F, Cl, Br, or I), oxo (═O), hydroxy (—OH), methanoyl (—COH), carboxy (—CO₂H), nitro (—NO₂), —NH₂, —N(CH₃)₂, cyano (—CN), ethynyl (—CCH), propynyl, sulfo (—SO₃H), morpholinyl, —CO-morpholin-4-yl, phenyl, —CONH₂, —CON(CH₃)₂, C₁-C₃ alkyl, C₁-C₃ perfluorinated alkyl, —CF₃, —OCF₃, or C₁-C₃ alkoxy) or unsubstituted phenyl, pyridinyl, pyrimidinyl, or pyrazinyl. In some embodiments, A is an unsubstituted phenyl or pyrazinyl. In certain embodiments, A is

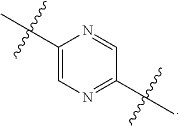 [SMILES: *c1cnc(*)cn1]

In some embodiments, n is 0, 1, 2, or 3. In other embodiments n is 0 or 1.

In some embodiments, R⁹ can be ortho, para or meta to the other connecting carbon on the phenyl, and can be H, halogen (e.g., F, Cl, Br, or I), hydroxy (—OH), methanoyl (—COH), carboxy (—CO₂H), nitro (—NO₂), —NH₂, —N(CH₃)₂, cyano (—CN), ethynyl (—CCH), propynyl, sulfo (—SO₃H), morpholinyl, —CO-morpholin-4-yl, —O—CH₂-heteroaryl, —O—CH₂-heterocyclyl, —O—CH₂-phenyl, —O—CH₂-pyridinyl, —O—CH₂-pyrimidinyl, —O—CH₂-pyrazinyl, —CONH₂, —CON(CH₃)₂, C₁-C₅ alkyl, C₁-C₃ alkyl, C₅-C₃ perfluorinated alkyl, —CF₃, —OCF₃, C₁-C₅ alkoxy, C₁-C₃ alkoxy, —O-phenyl, methyl, ethyl, propyl, cycloalkyl, heterocyclyl, aryl, heteroaryl, phenyl, pyridinyl, pyrimidinyl, pyrazinyl, —O(CO)H, —O(CO)(C₁-C₅ alkyl), —NH(CO)(C₁-C₅ alkyl), —N(C₁-C₅ alkyl)₂, —NH(C₁-C₅ alkyl), —O(CO)(C₁-C₃ alkyl), —NH(CO)(C₁-C₃ alkyl), —N(C₁-C₃ alkyl)₂, —NH(C₁-C₃ alkyl), —O(CO)(phenyl), —NH(CO)(phenyl), —N(C₁-C₃ alkyl)(phenyl), or —NH(phenyl). In other embodiments, R⁹ can be ortho, para or meta to the other connecting carbon on the phenyl, and can be H, halogen (e.g., F, Cl, Br, or I), hydroxy (—OH), methanoyl (—COH), carboxy (—CO₂H), nitro (—NO₂), —NH₂, —N(CH₃)₂, cyano (—CN), sulfo (—SO₃H), —O—CH₂-heteroaryl, —O—CH₂-heterocyclyl, —O—CH₂-phenyl, —O—CH₂-pyridinyl, —O—CH₂-pyrimidinyl, —O—CH₂-pyrazinyl, —CONH₂, —CON(CH₃)₂, C₁-C₅ alkyl, C₁-C₃ alkyl, C₁-C₃ perfluorinated alkyl, —CF₃, —OCF₃, C₁-C₅ alkoxy, C₁-C₃ alkoxy, —O-phenyl, methyl, ethyl, propyl, cycloalkyl, heterocyclyl, aryl, heteroaryl, phenyl, pyridinyl, pyrimidinyl, pyrazinyl, —O(CO)H, —O(CO)(C₁-C₅ alkyl), —NH(CO)(C₁-C₅ alkyl), —N(C₁-C₅ alkyl)₂, —NH(C₁-C₅ alkyl), —O(CO)(C₁-C₃ alkyl), —NH(CO)(C₁-C₃ alkyl), —N(C₁-C₃ alkyl)₂, —NH(C₁-C₃ alkyl), —O(CO)(phenyl), —NH(CO)(phenyl), —N(C₁-C₃ alkyl)(phenyl), or —NH(phenyl). In yet other embodiments, R⁹ can be ortho, para or meta to the other connecting carbon on the phenyl, and can be H, halogen (e.g., F, Cl, Br, or I), hydroxy (—OH), methanoyl (—COH), carboxy (—CO₂H), nitro (—NO₂), —NH₂, —N(CH₃)₂, cyano (—CN), sulfo (—SO₃H), —CONH₂, —CON(CH₃)₂, C₁-C₅ alkyl, C₁-C₃ alkyl, C₁-C₅ alkoxy, C₁-C₃ alkoxy, —O— phenyl, methyl, ethyl, propyl, —CF₃, phenyl, pyridinyl, pyrimidinyl, pyrazinyl, —O(CO)H, —O(CO)(C₁-C₅ alkyl), —NH(CO)(C₁-C₅ alkyl), —N(C₁-C₅ alkyl)₂, —NH(C₁-C₅ alkyl), —O(CO)(C₁-C₃ alkyl), —NH(CO)(C₁-C₃ alkyl), —N(C₁-C₃ alkyl)₂, —NH(C₁-C₃ alkyl), —O(CO)(phenyl), —NH(CO)(phenyl), —N(C₁-C₃ alkyl)(phenyl), or —NH(phenyl). In yet other embodiments, R⁹ can be ortho, para or meta to the other connecting carbon on the phenyl, and can be H, halogen (e.g., F, Cl, Br, or I), hydroxy (—OH), cyano (—CN), C₁-C₅ alkyl, C₁-C₃ alkyl, C₁-C₅ alkoxy, C₁-C₃ alkoxy, methyl, ethyl, propyl, methoxy, ethoxy, —CF₃, —O(CO)H, —O(CO)(C₁-C₅ alkyl), —NH(CO)(C₁-C₅ alkyl), —O(CO)(C₁-C₃ alkyl), —NH(CO)(C₁-C₃ alkyl), —O(CO)CH₃, or —NH—(CO)—CH₃. In still other embodiments, R⁹ can be ortho, para or meta to the other connecting carbon on the phenyl, and can be H, F, Cl, Br, hydroxy (—OH), cyano (—CN), methyl, ethyl, methoxy, ethoxy, —CF₃, —O(CO)CH₃, or —NH—(CO)—CH₃.

In some embodiments, the compounds of Formula (I) can be selected from those specified in Table 1.

TABLE 1

| Compound | |
|---|---|
| Number | Compound Structure |
| I-1 | 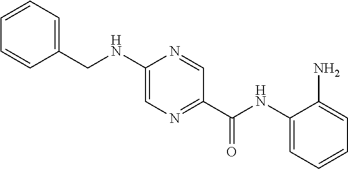 [SMILES: Nc1ccccc1NC(=O)c1cnc(NCc2ccccc2)cn1] |
| I-2 | 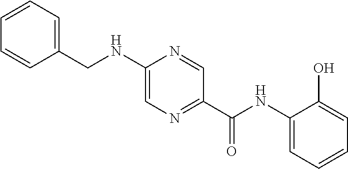 [SMILES: O=C(Nc1ccccc1O)c1cnc(NCc2ccccc2)cn1] |
| I-3 | 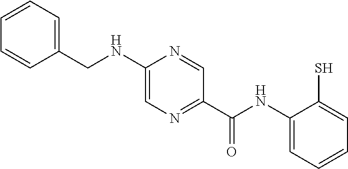 [SMILES: O=C(Nc1ccccc1S)c1cnc(NCc2ccccc2)cn1] |
| I-4 | 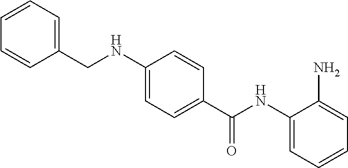 [SMILES: Nc1ccccc1NC(=O)c1ccc(NCc2ccccc2)cc1] |
| I-5 | 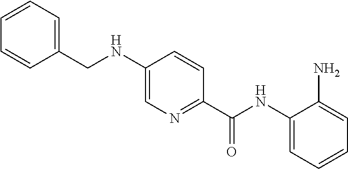 [SMILES: Nc1ccccc1NC(=O)c1ccc(NCc2ccccc2)cn1] |
| I-6 | 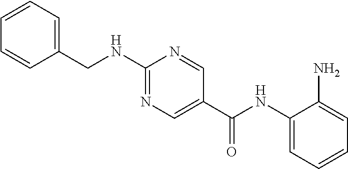 [SMILES: Nc1ccccc1NC(=O)c1cnc(NCc2ccccc2)nc1] |
| I-7 | 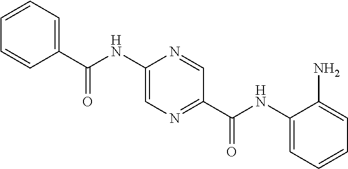 [SMILES: Nc1ccccc1NC(=O)c1cnc(NC(=O)c2ccccc2)cn1] |
| I-8 | 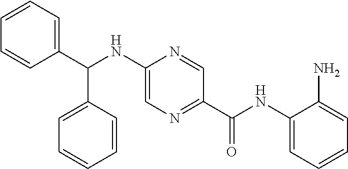 [SMILES: Nc1ccccc1NC(=O)c1cnc(NC(c2ccccc2)c2ccccc2)cn1] |
| I-9 | 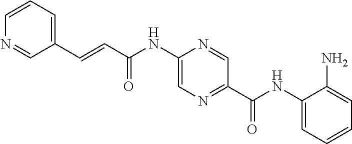 [SMILES: Nc1ccccc1NC(=O)c1cnc(NC(=O)/C=C/c2cccnc2)cn1] |
| I-10 | 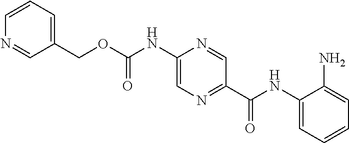 [SMILES: Nc1ccccc1NC(=O)c1cnc(NC(=O)OCc2cccnc2)cn1] |
| I-11 | 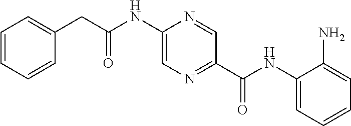 [SMILES: Nc1ccccc1NC(=O)c1cnc(NC(=O)Cc2ccccc2)cn1] |
| I-12 | 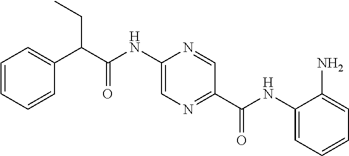 [SMILES: CCC(C(=O)Nc1cnc(C(=O)Nc2ccccc2N)cn1)c1ccccc1] |
| I-13 | 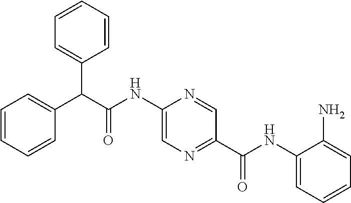 [SMILES: Nc1ccccc1NC(=O)c1cnc(NC(=O)C(c2ccccc2)c2ccccc2)cn1] |
| I-14 | 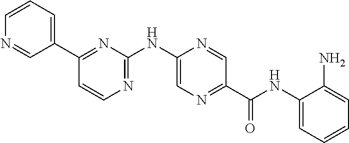 [SMILES: Nc1ccccc1NC(=O)c1cnc(Nc2nccc(-c3cccnc3)n2)cn1] |
| I-15 | 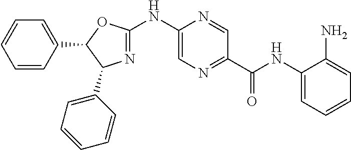 [SMILES: Nc1ccccc1NC(=O)c1cnc(NC2=N[C@H](c3ccccc3)[C@H](c3ccccc3)O2)cn1] |
| I-16 | 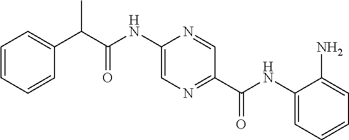 [SMILES: CC(C(=O)Nc1cnc(C(=O)Nc2ccccc2N)cn1)c1ccccc1] |
| I-17 | 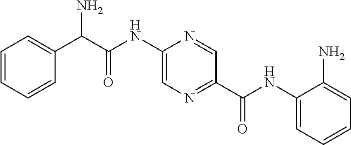 [SMILES: Nc1ccccc1NC(=O)c1cnc(NC(=O)C(N)c2ccccc2)cn1] |
| I-18 | 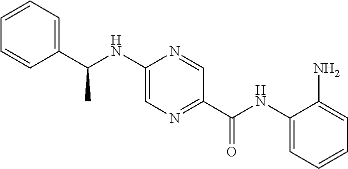 [SMILES: C[C@H](Nc1cnc(C(=O)Nc2ccccc2N)cn1)c1ccccc1] |
| I-19 | 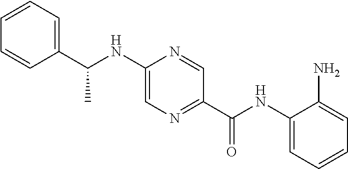 [SMILES: C[C@@H](Nc1cnc(C(=O)Nc2ccccc2N)cn1)c1ccccc1] |
| I-20 | 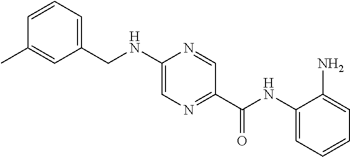 [SMILES: Cc1cccc(CNc2cnc(C(=O)Nc3ccccc3N)cn2)c1] |
| I-21 | 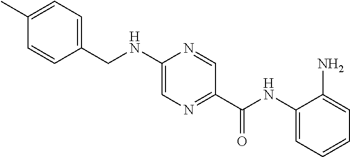 [SMILES: Cc1ccc(CNc2cnc(C(=O)Nc3ccccc3N)cn2)cc1] |
| I-22 | 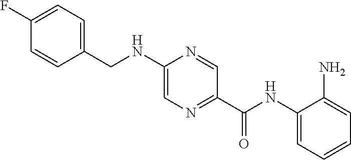 [SMILES: Nc1ccccc1NC(=O)c1cnc(NCc2ccc(F)cc2)cn1] |
| I-23 | 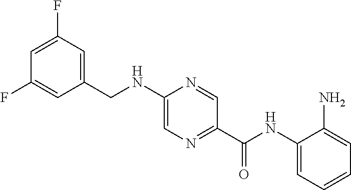 [SMILES: Nc1ccccc1NC(=O)c1cnc(NCc2cc(F)cc(F)c2)cn1] |
| I-24 | 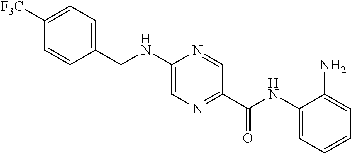 [SMILES: Nc1ccccc1NC(=O)c1cnc(NCc2ccc(C(F)(F)F)cc2)cn1] |
| I-25 | 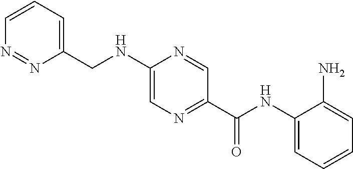 [SMILES: Nc1ccccc1NC(=O)c1cnc(NCc2cccnn2)cn1] |
| I-26 | 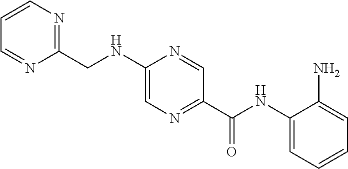 [SMILES: Nc1ccccc1NC(=O)c1cnc(NCc2ncccn2)cn1] |
| I-27 | 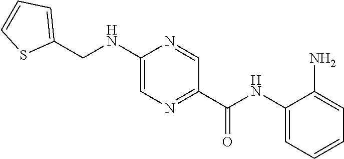 [SMILES: Nc1ccccc1NC(=O)c1cnc(NCc2cccs2)cn1] |

In some embodiments, one or more of compounds I-1, I-2, I-3, I-4, I-5, I-6, I-7, I-8, I-9, I-10, I-11, I-12, I-13, I-14, I-15, I-16, I-17, I-18, I-19, I-20, I-21, I-22, I-23, I-24, I-25, I-26, or I-27 are excluded from the compounds of the invention (e.g., Formula (I) or Formula (Ia)).

In some embodiments, one or more of compounds I-1, I-7, I-9, I-10, I-11, I-12, or I-13 are excluded from the compounds of the invention (e.g., Formula (I) or Formula (Ia)). In some embodiments, one or more of compounds I-7, I-9, I-10, I-11, I-12, or I-13 are excluded from the compounds of the invention (e.g., Formula (I) or Formula (Ia)). In some embodiments, compound I-1 is excluded from the compounds of the invention (e.g., Formula (I) or Formula (Ia)).

In some embodiments, the compounds of the invention include one or more of I-1, I-2, I-3, I-4, I-5, I-6, I-7, I-8, I-9, I-10, I-11, I-12, I-13, I-14, I-15, I-16, I-17, I-18, I-19, I-20, I-21, I-22, I-23, I-24, I-25, I-26, or I-27. In some embodiments, the compounds of the invention include one or more of I-1, I-2, I-3, I-4, I-5, I-6, I-8, I-14, I-15, I-16, I-17, I-18, I-19, I-20, I-21, I-22, I-23, I-24, I-25, I-26, or I-27. In some embodiments, the compounds of the invention include one or more of I-1, I-2, I-3, I-4, I-5, I-6, I-8, I-16, or I-17. In some embodiments, the compounds of the invention include one or more of I-2, I-3, I-4, I-5, I-6, I-8, or I-17.

In some embodiments, the compounds of the invention include I-1, I-2, I-3, I-4, I-5, I-6, I-7, I-8, I-9, I-10, I-11, I-12, I-13, I-14, I-15, I-16, I-17, I-18, I-19, I-20, I-21, I-22, I-23, I-24, I-25, I-26, and I-27. In some embodiments, the compounds of the invention include I-1, I-2, I-3, I-4, I-5, I-6, I-8, I-14, I-15, I-16, and I-17. In some embodiments, the compounds of the invention include I-1, I-2, I-3, I-4, I-5, I-6, I-8, and I-17. In some embodiments, the compounds of the invention include I-2, I-3, I-4, I-5, I-6, I-8, and I-17.

In some embodiments, the compounds of Formula (I) (e.g., Formula (Ia), I-1, I-8, or I-17) can be in the form of salts, optical and geometric isomers, and salts of isomers. In other embodiments, the compounds can be in various forms, such as uncharged molecules, components of molecular complexes, or non-irritating pharmacologically acceptable salts, including but not limited to hydrochloride, hydrobromide, sulphate, phosphate, nitrate, borate, acetate, maleate, tartrate, and salicylate. In some instances, for acidic compounds, salts can include metals, amines, or organic cations (e.g. quaternary ammonium). In yet other embodiments, derivatives of the compounds (e.g., ethers, esters, or amides) which have desirable retention and release characteristics, but which are hydrolyzed (e.g., easily hydrolyzed) by body pH, enzymes, or other suitable means, can be employed.

In some embodiments, the compounds of the invention having a chiral center and can exist in and be isolated in optically active and racemic forms. In other embodiments, compounds may exhibit polymorphism. Some embodiments of the present invention encompass any racemic, optically active, polymorphic, or stereoisomeric form, or mixtures thereof, of a compound described herein. The preparation of optically active forms can be accomplished by any suitable method, including but not limited to, resolution of the racemic form by recrystallization techniques, synthesis from optically-active starting materials, chiral synthesis, or chromatographic separation using a chiral stationary phase.

In other embodiments, compounds of the invention encompass Formula (I) and the salts, optical isomers, geometric isomers, salts of isomers, and derivatives (e.g., ethers, esters, or amides) thereof. In yet other embodiments, compounds of the invention encompass Formula (Ia) and the salts, optical isomers, geometric isomers, salts of isomers, and derivatives (e.g., ethers, esters, or amides) thereof.

In some embodiments, one or more compounds disclosed herein will lead to an increase in myelination (e.g., increasing myelination on the axon sheath). In other embodiments, one or more compounds disclosed herein can lead to axon regrowth. In certain embodiments, one or more compounds disclosed herein can inhibit (e.g., fully inhibit or partially inhibit) one or more of HDAC3, HDAC, demethylase, and methyltransferase by, for example, reducing the activity or expression of an enzyme (e.g., HDAC3, HDAC, demethylase, or methyltransferase). In other embodiments, by one or more compounds disclosed herein (e.g., an HDAC3 inhibitor) can be antagonists (e.g., antagonists of one or more of HDAC3, HDAC, demethylase, and methyltransferase), partial antagonists (e.g., partial antagonists of one or more of HDAC3, HDAC, demethylase, and methyltransferase), inverse agonists (e.g., inverse antagonists of one or more of HDAC3, HDAC, demethylase, and methyltransferase), partial inverse agonists (e.g., partial inverse antagonists of one or more of HDAC3, HDAC, demethylase, and methyltransferase), or combinations thereof. In certain embodiments, inhibition by one or more compounds disclosed herein can occur using any suitable mechanism, such as but not limited to blockading an enzyme (e.g., partially or fully blocking other molecules from accessing one or more receptor sites), an antagonist mechanism, a partial antagonist mechanism, an inverse agonist mechanism, a partial inverse agonist mechanism, or a combination thereof.

Compositions

In certain embodiments, the compounds of the invention (e.g., Formula (I), Formula (Ia), I-1, I-8, or I-17) can be part of a composition and can be in an amount (by weight of the total composition) of at least about 0.0001%, at least about 0.001%, at least about 0.10%, at least about 0.15%, at least about 0.20%, at least about 0.25%, at least about 0.50%, at least about 0.75%, at least about 1%, at least about 10%, at least about 25%, at least about 50%, at least about 75%, at least about 90%, at least about 95%, at least about 99%, at least about 99.99%, no more than about 75%, no more than about 90%, no more than about 95%, no more than about 99%, or no more than about 99.99%, from about 0.0001% to about 99%, from about 0.0001% to about 50%, from about 0.01% to about 95%, from about 1% to about 95%, from about 10% to about 90%, or from about 25% to about 75%.

In some embodiments, the compounds of the invention (e.g., Formula (I), Formula (Ia), I-1, I-8, or I-17) can be purified or isolated in an amount (by weight of the total composition) of at least about 0.0001%, at least about 0.001%, at least about 0.10%, at least about 0.15%, at least about 0.20%, at least about 0.25%, at least about 0.50%, at least about 0.75%, at least about 1%, at least about 10%, at least about 25%, at least about 50%, at least about 75%, at least about 90%, at least about 95%, at least about 99%, at least about 99.99%, no more than about 75%, no more than about 90%, no more than about 95%, no more than about 99%, no more than about 99.99%, from about 0.0001% to about 99%, from about 0.0001% to about 50%, from about 0.01% to about 95%, from about 1% to about 95%, from about 10% to about 90%, or from about 25% to about 75%.

Some embodiments of the present invention include compositions comprising the compounds of the invention (e.g., Formula (I), Formula (Ia), I-1, I-8, or I-17). In certain embodiments, the composition is a pharmaceutical composition, such as compositions that are suitable for administration to animals (e.g., mammals, primates, monkeys, humans, canine, feline, porcine, mice, rabbits, or rats). In some instances, the pharmaceutical composition is non-toxic, does not cause side effects, or both. In some embodiments, there may be inherent side effects (e.g., it may harm the patient or may be toxic or harmful to some degree in some patients).

“Therapeutically effective amount” means an amount effective to achieve a desired and/or beneficial effect. An effective amount can be administered in one or more administrations. For some purposes of this invention, a therapeutically effective amount is an amount appropriate to treat an indication (e.g., to treat disease, such as Multiple Sclerosis (MS), or nerve damage). By treating an indication is meant achieving any desirable effect, such as one or more of palliate, ameliorate, stabilize, reverse, slow, or delay disease (e.g., MS) progression, increase the quality of life, or to prolong life. Such achievement can be measured by any suitable method, such as but not limited to measurement of the extent of myelination (e.g., g ratio), extent of motor function (e.g., toe spreading, latency to fall), action potential, nerve function, nerve conduction velocity, nerve CMAP amplitude, nerve CMAP duration, number of myelinated axons per area, extent of axonal regrowth, clinical EAE score, an MS progression test (e.g., using one or more of Expanded Disability Status Scale, Functional System Score, or Multiple Sclerosis Functional Composite), or any suitable method to assess the progression of the disease, (e.g., MS) or nerve damage (e.g., CNS nerve damage or PNS nerve damage).

In some embodiments, the compounds of the invention (e.g., Formula (I), Formula (Ia), I-1, I-8, or I-17) can be part of a pharmaceutical composition and can be in an amount of at least about 0.0001%, at least about 0.001%, at least about 0.10%, at least about 0.15%, at least about 0.20%, at least about 0.25%, at least about 0.50%, at least about 0.75%, at least about 1%, at least about 10%, at least about 25%, at least about 50%, at least about 75%, at least about 90%, at least about 95%, at least about 99%, at least about 99.99%, no more than about 75%, no more than about 90%, no more than about 95%, no more than about 99%, no more than about 99.99%, from about 0.001% to about 99%, from about 0.001% to about 50%, from about 0.1% to about 99%, from about 1% to about 95%, from about 10% to about 90%, or from about 25% to about 75%. In some embodiments, the pharmaceutical composition can be presented in a dosage form which is suitable for the topical, subcutaneous, intrathecal, intraperitoneal, oral, parenteral, rectal, cutaneous, nasal, vaginal, or ocular administration route. In other embodiments, the pharmaceutical composition can be presented in a dosage form which is suitable for parenteral administration, a mucosal administration, intravenous administration, depot injection (e.g., solid or oil based), subcutaneous administration, topical administration, intradermal administration, oral administration, sublingual administration, intranasal administration, or intramuscular administration. The pharmaceutical composition can be in the form of, for example, tablets, capsules, pills, powders granulates, suspensions, emulsions, solutions, gels (including hydrogels), pastes, ointments, creams, plasters, drenches, delivery devices, suppositories, enemas, injectables, implants, sprays, aerosols or other suitable forms.

In some embodiments, the pharmaceutical composition can include one or more formulary ingredients. A “formulary ingredient” can be any suitable ingredient (e.g., suitable for the drug(s), for the dosage of the drug(s), for the timing of release of the drugs(s), for the disease, for the disease state, or for the delivery route) including, but not limited to, water (e.g., boiled water, distilled water, filtered water, pyrogen-free water, or water with chloroform), sugar (e.g., sucrose, glucose, mannitol, sorbitol, xylitol, or syrups made therefrom), ethanol, glycerol, glycols (e.g., propylene glycol), acetone, ethers, DMSO, surfactants (e.g., anionic surfactants, cationic surfactants, zwitterionic surfactants, or nonionic surfactants (e.g., polysorbates)), oils (e.g., animal oils, plant oils (e.g., coconut oil or arachis oil), or mineral oils), oil derivatives (e.g., ethyl oleate, glyceryl monostearate, or hydrogenated glycerides), excipients, preservatives (e.g., cysteine, methionine, antioxidants (e.g., vitamins (e.g., A, E, or C), selenium, retinyl palmitate, sodium citrate, citric acid, chloroform, or parabens, (e.g., methyl paraben or propyl paraben)), or combinations thereof. For example, parenteral administration, a mucosal administration, intravenous administration, depot injection (e.g., solid or oil based), subcutaneous administration, topical administration, intradermal administration, oral administration, sublingual administration, intranasal administration, or intramuscular administration, could include one or more formulary ingredients.

In certain embodiments, pharmaceutical compositions can be formulated to release the compounds of the invention (e.g., Formula (I), Formula (Ia), I-1, I-8, or I-17) substantially immediately upon the administration or any substantially predetermined time or time after administration. Such formulations can include, for example, controlled release formulations such as various controlled release compositions and coatings. For example, a parenteral administration, a mucosal administration, intravenous administration, depot injection (e.g., solid or oil based), subcutaneous administration, topical administration, intradermal administration, oral administration, sublingual administration, intranasal administration, or intramuscular administration, could be used for a controlled release (e.g., of the compounds of the invention, Formula (I), Formula (Ia), I-1, I-8, or I-17), and in some instances, could be administered once per hour (or once per day, several times per day, more than once per day, once per week, several times per week, once per three months, once per six months, or once per year).

Other formulations (e.g., formulations of a pharmaceutical composition) can, in certain embodiments, include those incorporating the drug (or control release formulation) into food, food stuffs, feed, or drink. For example, the compounds of the invention (e.g., Formula (I), Formula (Ia), I-1, I-8, or I-17) could be administered orally once per day, twice per day, three times per day, more than once per day, once per two days, or once per week.

Some embodiments of the invention can include methods of treating an organism for disease or nerve injury (e.g., CNS demyelinating disease, PNS demyelinating disease, MS, Alzheimer's Disease, amyotrophic lateral sclerosis (ALS), Huntington's Disease, CNS nerve injury, PNS nerve injury, crush nerve injury, or transection nerve injury). In certain embodiments, treating comprises administering the compounds of the invention (e.g., Formula (I), Formula (Ia), I-1, I-8, or I-17). In other embodiments, treating comprises administering the compounds of the invention (e.g., Formula (I), Formula (Ia), I-1, I-8, or I-17) to an animal that is effective to treat disease or nerve injury (e.g., CNS demyelinating disease, PNS demyelinating disease, MS, Alzheimer's Disease, amyotrophic lateral sclerosis (ALS), Huntington's Disease, CNS nerve injury, PNS nerve injury, crush nerve injury, or transection nerve injury). In some embodiments, a composition or pharmaceutical composition comprises the compounds of the invention (e.g., Formula (I), Formula (Ia), I-1, I-8, or I-17) which can be administered to an animal (e.g., mammals, primates, monkeys, or humans) in an amount of about 0.005 to about 100 mg/kg body weight, about 0.005 to about 50 mg/kg body weight, about 0.01 to about 15 mg/kg body weight, about 0.1 to about 10 mg/kg body weight, about 0.5 to about 7 mg/kg body weight, about 0.005 mg/kg, about 0.01 mg/kg, about 0.05 mg/kg, about 0.1 mg/kg, about 0.5 mg/kg, about 1.0 mg/kg, about 1.5 mg/kg, about 2.0 mg/kg, about 2.5 mg/kg, about 3.0 mg/kg, about 3.5 mg/kg, about 4.0 mg/kg, about 4.5 mg/kg, about 5 mg/kg, about 5.5 mg/kg, about 6 mg/kg, about 6.5 mg/kg, about 7 mg/kg, about 7.5 mg/kg, about 8 mg/kg, about 10 mg/kg, about 12 mg/kg, or about 15 mg/kg. In regard to some conditions, the dosage can be about 0.5 mg/kg human body weight, about 1.0 mg/kg human body weight, about 1.5 mg/kg human body weight, about 2.0 mg/kg human body weight, about 2.5 mg/kg human body weight, about 3.0 mg/kg human body weight, about 3.5 mg/kg human body weight, about 4.0 mg/kg human body weight, about 4.5 mg/kg human body weight, about 5.0 mg/kg human body weight, about 6.5 mg/kg human body weight, about 10 mg/kg human body weight, about 50 mg/kg human body weight, about 80 mg/kg human body weight, or about 100 mg/kg human body weight. In some instances, some animals (e.g., mammals, mice, rabbits, feline, porcine, or canine) can be administered a dosage of about 0.005 to about 200 mg/kg body weight, about 0.005 to about 50 mg/kg body weight, about 0.01 to about 15 mg/kg body weight, about 0.1 to about 10 mg/kg body weight, about 0.5 to about 7 mg/kg body weight, about 0.005 mg/kg, about 0.01 mg/kg, about 0.05 mg/kg, about 0.1 mg/kg, about 1.0 mg/kg, about 1.5 mg/kg, about 2.0 mg/kg, about 2.5 mg/kg, about 3.0 mg/kg, about 3.5 mg/kg, about 4.0 mg/kg, about 4.5 mg/kg, about 5.0 mg/kg, about 10 mg/kg, about 20 mg/kg, about 30 mg/kg, about 40 mg/kg, about 50 mg/kg, about 80 mg/kg, about 100 mg/kg, about 150 mg/kg, about 200 mg/kg, or about 250 mg/kg. Of course, those skilled in the art will appreciate that it is possible to employ many concentrations in the methods of the present invention, and using, in part, the guidance provided herein, will be able to adjust and test any number of concentrations in order to find one that achieves the desired result in a given circumstance. In other embodiments, the compounds of the invention (e.g., Formula (I), Formula (Ia), I-1, I-8, or I-17) can be administered in combination with one or more other therapeutic agents to treat a given disease or nerve injury (e.g., CNS demyelinating disease, PNS demyelinating disease, MS, Alzheimer's Disease, amyotrophic lateral sclerosis (ALS), Huntington's Disease, CNS nerve injury, PNS nerve injury, crush nerve injury, or transection nerve injury).

In some embodiments, the compositions can include a unit dose of one or more of the compounds of the invention (e.g., Formula (I), Formula (Ia), I-1, I-8, or I-17) in combination with a pharmaceutically acceptable carrier and, in addition, can include other medicinal agents, pharmaceutical agents, carriers, adjuvants, diluents, and excipients. In certain embodiments, the carrier, vehicle or excipient can facilitate administration, delivery and/or improve preservation of the composition. In other embodiments, the one or more carriers, include but are not limited to, saline solutions such as normal saline, Ringer's solution, PBS (phosphate-buffered saline), and generally mixtures of various salts including potassium and phosphate salts with or without sugar additives such as glucose. Carriers can include aqueous and non-aqueous sterile injection solutions that can contain antioxidants, buffers, bacteriostats, bactericidal antibiotics, and solutes that render the formulation isotonic with the bodily fluids of the intended recipient; and aqueous and non-aqueous sterile suspensions, which can include suspending agents and thickening agents. In other embodiments, the one or more excipients can include, but are not limited to water, saline, dextrose, glycerol, ethanol, or the like, and combinations thereof. Nontoxic auxiliary substances, such as wetting agents, buffers, or emulsifiers may also be added to the composition. Oral formulations can include such normally employed excipients as, for example, pharmaceutical grades of mannitol, lactose, starch, magnesium stearate, sodium saccharine, cellulose, and magnesium carbonate.

Administration Routes and Treatments of Disease and Injury

The compounds of the invention (e.g., Formula (I), Formula (Ia), I-1, I-8, or I-17) can be administered to animals by any number of suitable administration routes or formulations. The compounds of the invention (e.g., Formula (I), Formula (Ia), I-1, I-8, or I-17) can also be used to treat animals for a variety of diseases. Animals include but are not limited to mammals, primates, monkeys (e.g., macaque, rhesus macaque, or pig tail macaque), humans, canine, feline, bovine, porcine, avian (e.g., chicken), mice, rabbits, and rats. As used herein, the term “subject” refers to both human and animal subjects.

The route of administration of the compounds of the invention (e.g., Formula (I), Formula (Ia), I-1, I-8, or I-17) can be of any suitable route. Administration routes can be, but are not limited to the oral route, the parenteral route, the cutaneous route, the nasal route, the rectal route, the vaginal route, and the ocular route. In other embodiments, administration routes can be parenteral administration, a mucosal administration, intravenous administration, subcutaneous administration, topical administration, intradermal administration, oral administration, sublingual administration, intranasal administration, or intramuscular administration. The choice of administration route can depend on the compound identity (e.g., the physical and chemical properties of the compound) as well as the age and weight of the animal, the particular disease or injury (e.g., in the CNS or PNS; transection vs. crushing injury), and the severity of the disease or injury (e.g., stage or severity of disease or injury). Of course, combinations of administration routes can be administered, as desired.

Some embodiments of the invention include a method for providing a subject with a composition comprising one or more compounds of the invention (e.g., Formula (I), Formula (Ia), I-1, I-8, or I-17) described herein (e.g., a pharmaceutical composition) which comprises one or more administrations of one or more such compositions; the compositions may be the same or different if there is more than one administration. Some embodiments of the invention include treatment of disease, nerve injury, or both in an animal comprising administering a compound of the invention (e.g., Formula (I), Formula (Ia), I-1, I-8, or I-17) resulting in inhibiting HDAC3, inhibiting HDAC, inhibiting demethylase, or inhibiting methyltransferase.

Some embodiments of the invention include treatment of disease, treatment of nerve injury (e.g., to the central nervous system (CNS) or the peripheral nervous system (PNS)), or both in an animal comprising administering a compound of the invention (e.g., Formula (I), Formula (Ia), I-1, I-8, or I-17). Administration to the animal can be accomplished by any number of suitable administration routes or formulations. Animals include but are not limited to mammals, primates, monkeys (e.g., macaque, rhesus macaque, or pig tail macaque), humans, canine, feline, bovine, porcine, avian (e.g., chicken), mice, rabbits, and rats. As used herein, the term “subject” refers to both human and animal subjects.

In some embodiments the age of the animal can be young or old. In other embodiments, the age of the animal (e.g., human) can be about 0.1, about 0.5, about 1, about 2, about 3, about 4, about 5, about 10, about 15, about 20, about 25, about 30, about 35, about 40, about 45, about 50, about 55, about 60, about 65, about 70, about 75, about 80, about 85, about 90, about 95, about 100, about 105, about 110, about 115, about 120, about 125, or about 150 years old. In certain embodiments, the animal can be no more than about 2 years old, no more than about 5 years old, no more than about 10 years old, no more than about 20 years old, at least about 40 years old, at least about 50 years old, at least about 65 years old, at least about 80 years old, or at least about 100 years old.

In some embodiments, the amount of a compound of the invention (e.g., Formula (I), Formula (Ia), I-1, I-8, or I-17) administered to an animal (e.g., via a composition or a pharmaceutical composition) can be, but is not limited to about 0.005 mg/kg, about 0.01 mg/kg, about 0.05 mg/kg, about 0.1 mg/kg, about 0.5 mg/kg, about 0.6 mg/kg, about 0.7 mg/kg, about 0.8 mg/kg, about 0.9 mg/kg, about 1.0 mg/kg, about 1.1 mg/kg, about 1.2 mg/kg, about 1.3 mg/kg, about 1.4 mg/kg, about 1.5 mg/kg, about 1.6 mg/kg, about 1.7 mg/kg, about 1.8 mg/kg, about 1.9 mg/kg, about 2.0 mg/kg, about 2.1 mg/kg, about 2.2 mg/kg, about 2.3 mg/kg, about 2.4 mg/kg, about 2.5 mg/kg, about 2.6 mg/kg, about 2.7 mg/kg, about 2.8 mg/kg, about 2.9 mg/kg, about 3.0 mg/kg, about 3.1 mg/kg, about 3.2 mg/kg, about 3.3 mg/kg, about 3.4 mg/kg, about 3.5 mg/kg, about 3.6 mg/kg, about 3.7 mg/kg, about 3.8 mg/kg, about 3.9 mg/kg, about 4.0 mg/kg, about 4.1 mg/kg, about 4.2 mg/kg, about 4.3 mg/kg, about 4.4 mg/kg, about 4.5 mg/kg, about 5.0 mg/kg, about 5.5 mg/kg, about 6.0 mg/kg, about 6.5 mg/kg, about 7.0 mg/kg, about 7.5 mg/kg, about 10 mg/kg, about 20 mg/kg, about 30 mg/kg, about 40 mg/kg, about 50 mg/kg, about 80 mg/kg, about 100 mg/kg, about 150 mg/kg, about 200 mg/kg, about 250 mg/kg, no more than about 20.0 mg/kg, no more than about 10.0 mg/kg, no more than about 5.0 mg/kg, no more than about 4.5 mg/kg, no more than about 4.4 mg/kg, no more than about 4.3 mg/kg, no more than about 4.2 mg/kg, no more than about 4.1 mg/kg, no more than about 4.0 mg/kg, no more than about 3.9 mg/kg, no more than about 3.8 mg/kg, no more than about 3.7 mg/kg, no more than about 3.6 mg/kg, no more than about 3.5 mg/kg, no more than about 3.4 mg/kg, no more than about 3.3 mg/kg, no more than about 3.2 mg/kg, no more than about 3.1 mg/kg, no more than about 3.0 mg/kg, no more than about 2.9 mg/kg, no more than about 2.8 mg/kg, no more than about 2.7 mg/kg, no more than about 2.6 mg/kg, no more than about 2.5 mg/kg, no more than about 2.4 mg/kg, no more than about 2.3 mg/kg, no more than about 2.2 mg/kg, no more than about 2.1 mg/kg, no more than about 2.0 mg/kg, no more than about 1.9 mg/kg, no more than about 1.8 mg/kg, no more than about 1.7 mg/kg, no more than about 1.6 mg/kg, no more than about 1.5 mg/kg, no more than about 1.4 mg/kg, no more than about 1.3 mg/kg, no more than about 1.2 mg/kg, no more than about 1.1 mg/kg, no more than about 1.0 mg/kg, no more than about 0.9 mg/kg, no more than about 0.8 mg/kg, no more than about 0.7 mg/kg, no more than about 0.6 mg/kg, or no more than about 0.5 mg/kg animal body weight. The animal (e.g., human) body weight can be about 2 kg, about 5 kg, about 10 kg, about 15 kg, about 20 kg, about 25 kg, about 30 kg, about 35 kg, about 40 kg, about 45 kg, about 50 kg, about 55 kg, about 60 kg, about 65 kg, about 70 kg, about 75 kg, about 80 kg, about 85 kg, about 90 kg, about 95 kg, about 100 kg, about 150 kg, about 200 kg, from about 2 kg to about 200 kg, from about 10 kg to about 100 kg, from about 10 kg to about 85 kg, from about 45 kg to about 100 kg, or from about 45 kg to about 85 kg. These amounts (e.g., dosages) can be used as an effective amount or a therapeutically effective amount.

Nerve injuries (e.g., from disease, crushing injury, or transection injury) to the CNS or nerve injury to the PNS (e.g., from disease, crushing injury, or transection injury) that can be treated in an animal (e.g., mammals, porcine, canine, avian (e.g., chicken), bovine, feline, primates, rodents, monkeys, rabbits, mice, rats, and humans) using a compound of the invention (e.g., Formula (I), Formula (Ia), I-1, I-8, or I-17) include, but are not limited to repairing nerve damage (e.g., in the CNS or PNS), improving nerve function (e.g., in the CNS or PNS), improving action potential (e.g., in the CNS or PNS), re-connecting axons (e.g., in the CNS or PNS), repairing axons (e.g., in the CNS or PNS), promoting myelination (e.g., in the CNS or PNS), increasing the extent of myelination (e.g., in the CNS or PNS), increasing the extent of myelination on the myelin sheath (e.g., in the CNS or PNS), reducing inflammation (e.g., in the CNS or PNS), reducing inflammation near (e.g., no more than about 1 mm, no more than about 3 mm, no more than about 5 mm, or no more than about 10 mm) or at an axon (e.g., in the CNS or PNS), traumatic brain injury, acquired brain injury (e.g., hypoxic ischemic brain injury), strokes, or periventricular leukomalacia (PVL; e.g., white-matter brain injury).

Diseases that can be treated in an animal (e.g., mammals, porcine, canine, avian (e.g., chicken), bovine, feline, primates, rodents, monkeys, rabbits, mice, rats, and humans) using a compound of the invention (e.g., Formula (I), Formula (Ia), I-1, I-8, or I-17) include, but are not limited to myelopathy (e.g., spinal cord injury, myelitis, vascular myelopathy, cervical spondylotic myelopathy, spondylosis, spinal stenosis), demyelinating disease (e.g., any disease of the nervous system where the myelin sheath of a neuron is damaged), CNS demyelinating disease, PNS demyelinating disease, genetic demyelinating disease, infectious demyelinating disease, autoimmune demyelinating disease, demyelinating myelinoclastic disease, demyelinating leukodystrophic disease, Devic's disease, CNS neuropathies (e.g., diseases resulting in vitamin B12 deficiency), central pontine myelinolysis, myelopathies (e.g., tabes dorsalis), leukoencephalopathies (e.g., progressive multifocal leukoencephalopathy), leukodystrophies, optic neuritis, transverse myelitis, neuromyelitis optica, Guillain-Barré syndrome, chronic inflammatory demyelinating polyneuropathy, anti-MAG peripheral neuropathy, Charcot-Marie-Tooth disease, Hereditary neuropathy with liability to pressure palsy, copper deficiency associated conditions (e.g., peripheral neuropathy, myelopathy, and optic neuropathy), progressive inflammatory neuropathy, multiple sclerosis (MS) (e.g., treating inflammation, remyelination, or both), MS-type clinically isolated syndrome (e.g., treating inflammation, remyelination, or both), relapsing-remitting MS (e.g., treating inflammation, remyelination, or both), primary progressive MS (e.g., treating inflammation, remyelination, or both), secondary progressive MS (e.g., treating inflammation, remyelination, or both), Alzheimer's Disease, amyotrophic lateral sclerosis (ALS), and Huntington's Disease, traumatic brain injury, acquired brain injury (e.g., hypoxic ischemic brain injury), strokes, or periventricular leukomalacia (PVL; e.g., white-matter brain injury).

The route of administration for treatment can be of any suitable route. Administration routes can be, but are not limited to the oral route, the parenteral route, the cutaneous route, the nasal route, the rectal route, the vaginal route, and the ocular route. In other embodiments, the administration route can be parenteral administration, a mucosal administration, intravenous administration, depot injection, subcutaneous administration, topical administration, intradermal administration, oral administration, sublingual administration, intranasal administration, or intramuscular administration. The choice of administration route can depend on the compound identity (e.g., the physical and chemical properties of the compound) as well as the age and weight of the animal, the particular disease or injury (e.g., in the CNS or PNS; transection vs. crushing injury), and the severity of the disease or injury (e.g., stage or severity of disease or injury). Of course, combinations of administration routes can be administered, as desired.

Some embodiments of the invention include a method for providing a subject with a composition comprising a compound of the invention (e.g., Formula (I), Formula (Ia), I-1, I-8, or I-17) (e.g., a pharmaceutical composition) which comprises one or more administrations of one or more such compositions; the compositions may be the same or different if there is more than one administration.

Animals that can be treated include but are not limited to mammals, rodents, primates, monkeys (e.g., macaque, rhesus macaque, pig tail macaque), humans, canine, feline, porcine, avian (e.g., chicken), bovine, mice, rabbits, and rats. As used herein, the term “subject” refers to both human and animal subjects. In some instances, the animal is in need of the treatment (e.g., by showing signs of disease or nerve injury).

In some embodiments, diseases or nerve injuries that can be treated in an animal (e.g., mammals, porcine, canine, avian (e.g., chicken), bovine, feline, primates, rodents, monkeys, rabbits, mice, rats, and humans) using a compound of the invention (e.g., Formula (I), Formula (Ia), I-1, I-8, or I-17) (e.g., by a composition comprising a compound of the invention, such as Formula (I), Formula (Ia), I-1, I-8, or I-17) include, but are not limited to the nerve injuries described herein and the diseases described herein.

As used herein, the term “treating” (and its variations, such as “treatment”) is to be considered in its broadest context. In particular, the term “treating” does not necessarily imply that an animal is treated until total recovery. Accordingly, “treating” includes amelioration of the symptoms, relief from the symptoms or effects associated with a condition, decrease in severity of a condition, or preventing, preventively ameliorating symptoms, or otherwise reducing the risk of developing a particular condition. As used herein, reference to “treating” an animal includes but is not limited to prophylactic treatment and therapeutic treatment. Any of the compositions (e.g., pharmaceutical compositions) described herein can be used to treat an animal.

As related to treating disease or nerve injury (e.g., CNS demyelinating disease, PNS demyelinating disease, MS, Alzheimer's Disease, amyotrophic lateral sclerosis (ALS), Huntington's Disease, CNS nerve injury, PNS nerve injury, crush nerve injury, or transection nerve injury), treating can include but is not limited to prophylactic treatment and therapeutic treatment. As such, treatment can include, but is not limited to: preventing disease or nerve injury (e.g., CNS demyelinating disease, PNS demyelinating disease, MS, Alzheimer's Disease, amyotrophic lateral sclerosis (ALS), Huntington's Disease, CNS nerve injury, PNS nerve injury, crush nerve injury, or transection nerve injury); reducing the risk of disease or nerve injury (e.g., CNS demyelinating disease, PNS demyelinating disease, MS, Alzheimer's Disease, amyotrophic lateral sclerosis (ALS), Huntington's Disease, CNS nerve injury, PNS nerve injury, crush nerve injury, or transection nerve injury); ameliorating or relieving symptoms of disease or nerve injury (e.g., CNS demyelinating disease, PNS demyelinating disease, MS, Alzheimer's Disease, amyotrophic lateral sclerosis (ALS), Huntington's Disease, CNS nerve injury, PNS nerve injury, crush nerve injury, or transection nerve injury); eliciting a bodily response against disease or nerve injury (e.g., CNS demyelinating disease, PNS demyelinating disease, MS, Alzheimer's Disease, amyotrophic lateral sclerosis (ALS), Huntington's Disease, CNS nerve injury, PNS nerve injury, crush nerve injury, or transection nerve injury); inhibiting the development or progression of disease or nerve injury (e.g., CNS demyelinating disease, PNS demyelinating disease, MS, Alzheimer's Disease, amyotrophic lateral sclerosis (ALS), Huntington's Disease, CNS nerve injury, PNS nerve injury, crush nerve injury, or transection nerve injury); inhibiting or preventing the onset of symptoms associated with disease or nerve injury (e.g., CNS demyelinating disease, PNS demyelinating disease, MS, Alzheimer's Disease, amyotrophic lateral sclerosis (ALS), Huntington's Disease, CNS nerve injury, PNS nerve injury, crush nerve injury, or transection nerve injury); reducing the severity of disease or nerve injury (e.g., CNS demyelinating disease, PNS demyelinating disease, MS, Alzheimer's Disease, amyotrophic lateral sclerosis (ALS), Huntington's Disease, CNS nerve injury, PNS nerve injury, crush nerve injury, or transection nerve injury); causing a regression of disease or nerve injury (e.g., CNS demyelinating disease, PNS demyelinating disease, MS, Alzheimer's Disease, amyotrophic lateral sclerosis (ALS), Huntington's Disease, CNS nerve injury, PNS nerve injury, crush nerve injury, or transection nerve injury) or one or more of the symptoms associated with disease or nerve injury (e.g., CNS demyelinating disease, PNS demyelinating disease, MS, Alzheimer's Disease, amyotrophic lateral sclerosis (ALS), Huntington's Disease, CNS nerve injury, PNS nerve injury, crush nerve injury, or transection nerve injury); causing remission of disease or nerve injury (e.g., CNS demyelinating disease, PNS demyelinating disease, MS, Alzheimer's Disease, amyotrophic lateral sclerosis (ALS), Huntington's Disease, CNS nerve injury, PNS nerve injury, crush nerve injury, or transection nerve injury); or preventing relapse of disease or nerve injury (e.g., CNS demyelinating disease, PNS demyelinating disease, MS, Alzheimer's Disease, amyotrophic lateral sclerosis (ALS), Huntington's Disease, CNS nerve injury, PNS nerve injury, crush nerve injury, or transection nerve injury). In some embodiments, treating does not include prophylactic treatment of one or both of disease or nerve injury (e.g., CNS demyelinating disease, PNS demyelinating disease, MS, Alzheimer's Disease, amyotrophic lateral sclerosis (ALS), Huntington's Disease, CNS nerve injury, PNS nerve injury, crush nerve injury, or transection nerve injury).

Treatment of an animal (e.g., human) can occur using any suitable administration method (such as those disclosed herein) and using any suitable amount of a compound of the invention (e.g., Formula (I), Formula (Ia), I-1, I-8, or I-17). In some embodiments, methods of treatment comprise treating an animal for disease or nerve injury (e.g., CNS demyelinating disease, PNS demyelinating disease, MS, Alzheimer's Disease, amyotrophic lateral sclerosis (ALS), Huntington's Disease, CNS nerve injury, PNS nerve injury, crush nerve injury, or transection nerve injury). Some embodiments of the invention include a method for treating a subject (e.g., an animal such as a human or primate) with a composition comprising a compound of the invention (e.g., Formula (I), Formula (Ia), I-1, I-8, or I-17) (e.g., a pharmaceutical composition) which comprises one or more administrations of one or more such compositions; the compositions may be the same or different if there is more than one administration.

In some embodiments, the method of treatment includes administering an effective amount of a composition comprising a compound of the invention (e.g., Formula (I), Formula (Ia), I-1, I-8, or I-17). As used herein, the term “effective amount” refers to a dosage or a series of dosages sufficient to affect treatment (e.g., to treat disease or nerve injury, e.g., CNS demyelinating disease, PNS demyelinating disease, MS, Alzheimer's Disease, amyotrophic lateral sclerosis (ALS), Huntington's Disease, CNS nerve injury, PNS nerve injury, crush nerve injury, or transection nerve injury) in an animal and include dosages disclosed herein (e.g., those disclosed above). In some embodiments, an effective amount can encompass a therapeutically effective amount, as disclosed herein. In certain embodiments, an effective amount can vary depending on the subject and the particular treatment being affected. The exact amount that is required can, for example, vary from subject to subject, depending on the age and general condition of the subject, the particular adjuvant being used (if applicable), administration protocol, and the like. As such, the effective amount can, for example, vary based on the particular circumstances, and an appropriate effective amount can be determined in a particular case. An effective amount can, for example, include any dosage or composition amount disclosed herein. In some embodiments, an effective amount of a compound of the invention (e.g., Formula (I), Formula (Ia), I-1, I-8, or I-17) (which can be administered to an animal such as mammals, primates, monkeys or humans) can be an amount of about 0.005 to about 50 mg/kg body weight, about 0.005 to about 80 mg/kg body weight, about 0.005 to about 100 mg/kg body weight, about 0.01 to about 15 mg/kg body weight, about 0.1 to about 10 mg/kg body weight, about 0.5 to about 7 mg/kg body weight, about 0.005 mg/kg, about 0.01 mg/kg, about 0.05 mg/kg, about 0.1 mg/kg, about 0.5 mg/kg, about 1.0 mg/kg, about 1.5 mg/kg, about 2.0 mg/kg, about 2.5 mg/kg, about 3.0 mg/kg, about 3.5 mg/kg, about 4.0 mg/kg, about 4.5 mg/kg, about 5.0 mg/kg, about 5.5 mg/kg, about 6 mg/kg, about 6.5 mg/kg, about 7.0 mg/kg, about 7.5 mg/kg, about 8.0 mg/kg, about 10 mg/kg, about 12 mg/kg, about 15 mg/kg, about 20 mg/kg, about 30 mg/kg, about 40 mg/kg, about 50 mg/kg, about 80 mg/kg, about 100 mg/kg, or about 150 mg/kg. In regard to some embodiments, the dosage can be about 0.1 mg/kg human body weight, about 0.5 mg/kg human body weight, about 1.0 mg/kg human body weight, about 1.5 mg/kg human body weight, about 2.0 mg/kg human body weight, about 2.5 mg/kg human body weight, about 3.0 mg/kg human body weight, about 3.5 mg/kg human body weight, about 4.0 mg/kg human body weight, about 4.5 mg/kg human body weight, about 5.0 mg/kg human body weight, about 10 mg/kg human body weight, about 50 mg/kg human body weight, about 80 mg/kg human body weight, about 100 mg/kg human body weight, or about 200 mg/kg human body weight. In some instances, an effective amount of a compound of the invention (e.g., Formula (I), Formula (Ia), I-1, I-8, or I-17) (which can be administered to an animal such as mammals, rodents, mice, rabbits, feline, porcine, or canine) can be an amount of about 0.005 to about 50 mg/kg body weight, about 0.005 to about 100 mg/kg body weight, about 0.005 to about 200 mg/kg body weight, about 0.01 to about 15 mg/kg body weight, about 0.1 to about 10 mg/kg body weight, about 0.5 to about 7 mg/kg body weight, about 0.005 mg/kg, about 0.01 mg/kg, about 0.05 mg/kg, about 0.1 mg/kg, about 0.5 mg/kg, about 1.0 mg/kg, about 1.5 mg/kg, about 2.0 mg/kg, about 2.5 mg/kg, about 3.0 mg/kg, about 3.5 mg/kg, about 4.0 mg/kg, about 4.5 mg/kg, about 5.0 mg/kg, about 5.5 mg/kg, about 6 mg/kg, about 6.5 mg/kg, about 7.0 mg/kg, about 7.5 mg/kg, about 8.0 mg/kg, about 10 mg/kg, about 20 mg/kg, about 30 mg/kg, about 40 mg/kg, about 50 mg/kg, about 80 mg/kg, about 100 mg/kg, about 150 mg/kg, about 200 mg/kg, or about 250 mg/kg. The amount of a compound of the invention (e.g., Formula (I), Formula (Ia), I-1, I-8, or I-17) can be any amount disclosed herein (e.g., an amount disclosed in the previous sentences). In some embodiments, an effective amount of a compound of the invention (e.g., Formula (I), Formula (Ia), I-1, I-8, or I-17) (which can be administered to an animal such as mammals, primates, monkeys or humans) can be an amount of about 1 to about 1000 mg/kg body weight, about 5 to about 500 mg/kg body weight, about 10 to about 200 mg/kg body weight, about 25 to about 100 mg/kg body weight, about 0.1 mg/kg, about 0.5 mg/kg, about 1.0 mg/kg, about 1.5 mg/kg, about 2.0 mg/kg, about 2.5 mg/kg, about 3.0 mg/kg, about 3.5 mg/kg, about 4.0 mg/kg, about 4.5 mg/kg, about 5.0 mg/kg, about 5.5 mg/kg, about 6 mg/kg, about 6.5 mg/kg, about 7.0 mg/kg, about 7.5 mg/kg, about 8.0 mg/kg, about 10 mg/kg, about 25 mg/kg, about 50 mg/kg, about 100 mg/kg, about 150 mg/kg, about 200 mg/kg, about 300 mg/kg, about 400 mg/kg, about 500 mg/kg, about 600 mg/kg, about 700 mg/kg, about 800 mg/kg, about 900 mg/kg, or about 1000 mg/kg. In regard to some conditions, the dosage can be about 0.1 mg/kg human body weight, about 0.5 mg/kg human body weight, about 1.0 mg/kg human body weight, about 1.5 mg/kg human body weight, about 2.0 mg/kg human body weight, about 2.5 mg/kg human body weight, about 3.0 mg/kg human body weight, about 3.5 mg/kg human body weight, about 4.0 mg/kg human body weight, about 4.5 mg/kg human body weight, about 5.0 mg/kg human body weight, about 10 mg/kg human body weight, about 20 mg/kg human body weight, about 80 mg/kg human body weight, or about 100 mg/kg human body weight. In some instances, an effective amount of a compound of the invention (e.g., Formula (I), Formula (Ia), I-1, I-8, or I-17) (which can be administered to an animal such as mammals, rodents, mice, rabbits, feline, porcine, or canine) can be an amount of about 1 to about 1000 mg/kg body weight, about 5 to about 500 mg/kg body weight, about 10 to about 200 mg/kg body weight, about 25 to about 100 mg/kg body weight, about 0.1 mg/kg, about 0.5 mg/kg, about 1.0 mg/kg, about 1.5 mg/kg, about 2.0 mg/kg, about 2.5 mg/kg, about 3.0 mg/kg, about 3.5 mg/kg, about 4.0 mg/kg, about 4.5 mg/kg, about 5.0 mg/kg, about 5.5 mg/kg, about 6 mg/kg, about 6.5 mg/kg, about 7.0 mg/kg, about 7.5 mg/kg, about 8.0 mg/kg, about 10 mg/kg, about 25 mg/kg, about 50 mg/kg, about 80 mg/kg, about 100 mg/kg, about 150 mg/kg, about 200 mg/kg, about 300 mg/kg, about 400 mg/kg, about 500 mg/kg, about 600 mg/kg, about 700 mg/kg, about 800 mg/kg, about 900 mg/kg, or about 1000 mg/kg.

“Therapeutically effective amount” means an amount effective to achieve a desired and/or beneficial effect (e.g., enhancing myelination). A therapeutically effective amount can be administered in one or more administrations. For some purposes of this invention, a therapeutically effective amount is an amount appropriate to treat an indication (e.g., to treat disease, such as MS, or nerve damage). By treating an indication is meant achieving any desirable effect, such as one or more of palliate, ameliorate, stabilize, reverse, slow, or delay disease (e.g., MS) progression, increase the quality of life, or to prolong life. Such achievement can be measured by any suitable method, such as but not limited to measurement of the extent of myelination (e.g., g ratio), extent of motor function (e.g., toe spreading, latency to fall), action potential, nerve function, nerve conduction velocity, nerve CMAP amplitude, nerve CMAP duration, number of myelinated axons per area, extent of axonal regrowth, clinical EAE score, an MS progression test (e.g., using one or more of Expanded Disability Status Scale, Functional System Score, or Multiple Sclerosis Functional Composite), or any suitable method to assess the progression of the disease, (e.g., MS) or nerve damage (e.g., CNS nerve damage or PNS nerve damage).

In some embodiments, other treatments are optionally included, and can be used with the inventive treatments described herein (e.g., administering a compound of the invention (e.g., Formula (I), Formula (Ia), I-1, I-8, or I-17)). Other treatments can comprise any known treatment (e.g., MS treatment) that is suitable to treat the disease or nerve injury. Some treatments can include related surgeries.

In some embodiments, additional optional treatments (e.g., as an “other treatment”) can also include one or more of surgical intervention, hormone therapies, immunotherapy, adjuvant systematic therapies, and MS therapies.

Methods for Preparing Compounds of Formula (I)

Some embodiments of the present invention include methods for the preparation of compounds of Formula (I) (e.g., Formula (Ia)). The compounds of Formula (I) (e.g., Formula (Ia)) can be prepared using any suitable method. In certain embodiments, a compound of Formula (I) (e.g., Formula (Ia)) can be prepared comprising the step of reacting a compound of Formula (II) with a compound of Formula (III) to result in Formula (IV), which is later made into Formula (I) (e.g., Formula (Ia)) (e.g., using one or more synthetic steps).

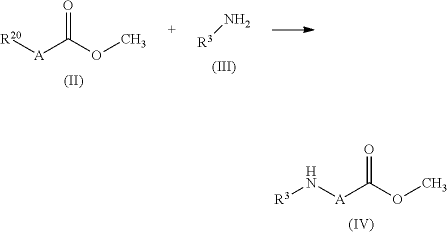

A and R³ of Formulas (II), (III), and (IV) are the same as that defined in Formula (I). R²⁰ is a halogen (e.g., Cl, Br, or I). Formula (II) can be prepared using any suitable method or can be purchased if available. Formula (III) can be prepared using any suitable method or can be purchased where available.

In some embodiments, Formula (II) can be reacted with Formula (III) under the following conditions: Formula (II) can be dissolved in any suitable solvent (e.g., 1,4-dioxane) and then Formula (III) can be added. The mole ratio of Formula (II) to Formula (III) can be any suitable mole ratio (e.g., about 2:1, about 1:1, or about 1:2). The mixture can be refluxed (e.g., at about 100° C.) for any suitable length of time (e.g., about 12 hours). Formula (IV) can then optionally be recovered using any suitable method.

In some embodiments, Formula (II) can be reacted with Formula (III) under the following conditions: Formula (II) (about 0.01 mmol) can be dissolved in 1,4-dioxane and then Formula (III) (about 0.01 mmol) can be added. The mole ratio of Formula (II) to Formula (III) can be about 1:1. The mixture can be refluxed at about 100° C. for about 12 hours. Formula (IV) can then optionally be recovered using any suitable method. Formula (IV) can be recovered (e.g., via extraction with ethyl acetate, dried over Na₂SO₄, filtered and evaporated, followed by further recovery using column chromatography).

In some embodiments, Formula (IV) can be reacted to provide Formula (V).

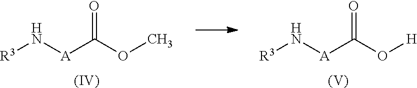

A and R³ of Formulas (II), (III), and (IV) are the same as that defined in Formula (I). Formula (IV) can be prepared using any suitable method (e.g., see above) or can be purchased if available.

In some embodiments, Formula (IV) can be reacted to provide Formula (V) under the following conditions: Formula (IV) can be added to any suitable solvent. An acid or a base (e.g., NaOH, KOH, or LiOH) can be dissolved in water, and then added to Formula (IV). Formula (V) can then optionally be recovered using any suitable method.

In some embodiments, Formula (IV) can be reacted to provide Formula (V) under the following conditions: Formula (IV) can be added to any suitable solvent (e.g., THF) and then stirred (e.g., for about 30 min.). LiOH can be dissolved in water, and then added to Formula (IV). The mole ratio of LiOH to Formula (IV) can be any suitable ratio (e.g., about 1:1, about 2:1, or about 3:1). The mixture can then be stirred for any suitable length of time (e.g., about overnight). The mixture can then optionally be neutralized with dilute HCl. Formula (V) can then optionally be recovered using any suitable method (e.g., extraction with ethyl acetate and dried).

In some embodiments, Formula (V) can be reacted with Formula (VI) to provide Formula (I) (e.g., Formula (Ia)).

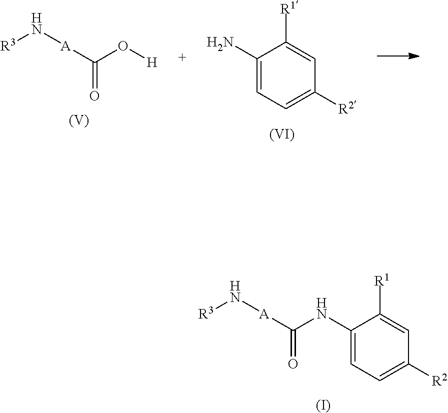

A, R¹, R², and R³ of Formula (V) are the same as that defined in Formula (I). Formula (V) can be prepared using any suitable method (e.g., see above) or can be purchased if available. Formula (VI) can be prepared using any suitable method (e.g., see above) or can be purchased if available. In some embodiments, R^{1′} and R^{2′} are R¹ and R², respectively (i.e., as defined in Formula (I)). In other embodiments, R^{1′} and/or R^{2′} are R¹ and/or R² (respectively) but with protecting groups (e.g., for amines, carbamates such as but not limited to t-butoxycarbonyl (Boc), benzyloxycarbonyl (Cbz), or 9-fluorenylmethoxycarbonyl (Fmoc)) to protect R¹ and/or R² during the reaction conditions with Formula (V). Where protecting group(s) are used, an additional step (or steps) of removing the protecting group(s) is used; any suitable protecting groups can be used and any suitable techniques for removing protecting groups can be used (e.g., using acid, such as about 4 M HCl).

In some embodiments, Formula (V) can be reacted with Formula (VI) to provide Formula (I) under the following conditions: Formula (V) can be dissolved in any suitable solvent (e.g., DCM:pyridine (1:1)). 1-(3-Dimethylaminopropyl)-3-ethylcarbodiimide hydrochloride (e.g., at about 1:3, about 1:2, about 1:1, about 2:1, or about 3:1 mole ratio to Formula (V)) can be added, and a catalytic amount of 4-(dimethylamino) pyridine (DMAP) can be added. Formula (VI) can be added (e.g., at about 1:2, about 1:1, or about 2:1 mole ratio to Formula (V)). Formula (I) (e.g., Formula (Ia)) can then optionally be recovered using any suitable method.

In some embodiments, Formula (V) can be reacted with Formula (VI) to provide Formula (I) under the following conditions: Formula (V) can be dissolved in any suitable solvent (e.g., DCM:pyridine (1:1)). 1-(3-Dimethylaminopropyl)-3-ethylcarbodiimide hydrochloride (e.g., at about 1:3, about 1:2, about 1:1, about 2:1, or about 3:1 mole ratio to Formula (V)) can be added, and a catalytic amount of 4-(dimethylamino) pyridine (DMAP) can be added. Formula (VI) can be added (e.g., at about 1:2, about 1:1, or about 2:1 mole ratio with Formula (V)). The mixture can then be flushed with an inert gas (e.g., nitrogen gas). The mixture can be then be stirred for a suitable amount of time (e.g., about 12 hours) at any suitable temperature (e.g., about room temperature). Formula (I) (e.g., Formula (Ia)) can then optionally be recovered using any suitable method, such as but not limited to evaporation, extraction/fractionation (e.g., with ethyl acetate and sodium bicarbonate) and/or purification with column chromatography.

Formula (I) (e.g., Formula (Ia)) can be optionally or further recovered. Recovery can occur using any suitable method including but not limited to HPLC (e.g., reverse phase), LC, filtration, precipitation, centrifugation, column chromatography (e.g., size exclusion chromatography or ion exchange chromatography), use of silica gel, washings (e.g., one or more time with one or more solvents or solvent mixtures), or combinations thereof.

In some embodiments, a method for the preparation of a compound of Formula (I) (e.g., Formula (Ia)) can comprise one or more of the above-mentioned steps. In certain embodiments, a method for preparing a compound of Formula (I) (e.g., Formula (Ia)) comprises (a) reacting a compound of Formula (II) with a compound of Formula (III) to result in a mixture comprising a compound of Formula (IV), (b) reacting a compound of Formula (IV) (e.g., with a base, such as but not limited to NaOH, KOH, or LiOH) to result in a mixture comprising a compound of Formula (V), and (c) reacting a compound of Formula (V) with a compound of Formula (VI) to result in a mixture comprising a compound of Formula (I) (e.g., Formula Ia). In some embodiments, protecting groups are used (e.g., in (c)), and such protecting groups are removed to result in a mixture comprising a compound of Formula (I) (e.g., Formula Ia). In other embodiments, the method further comprises recovering Formula (I) (e.g., Formula Ia).

The presently-disclosed subject matter is further illustrated by the following specific but non-limiting examples. The following examples may include compilations of data that are representative of data gathered at various times during the course of development and experimentation related to the present invention.

### EXAMPLES

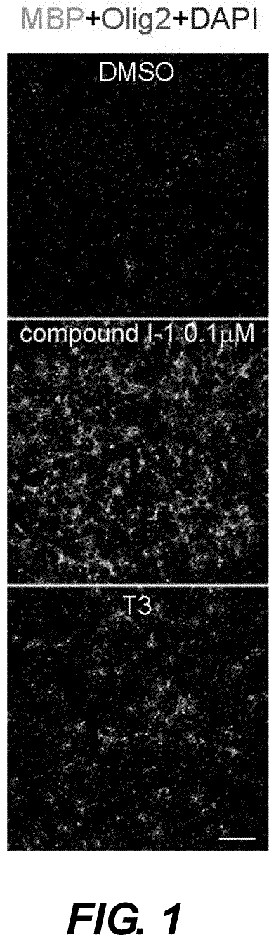

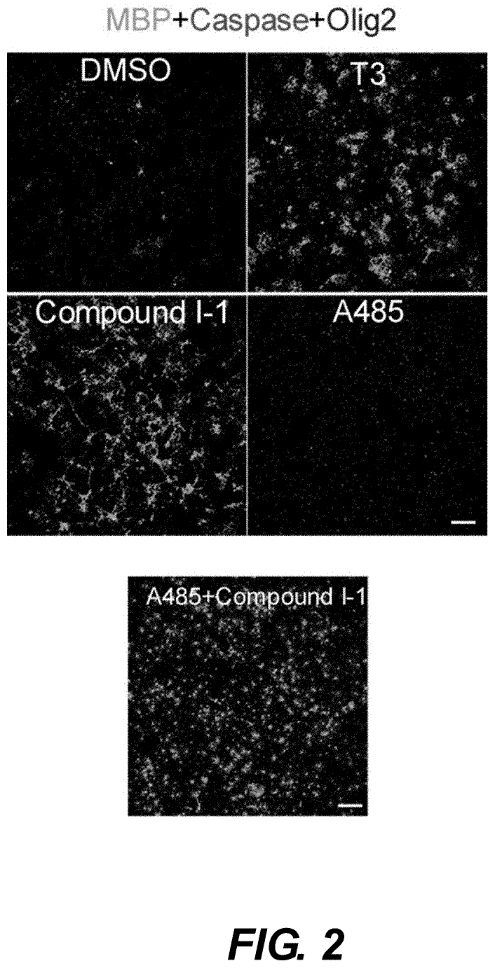

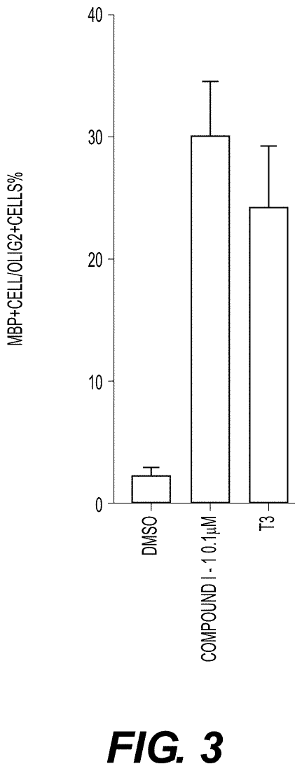

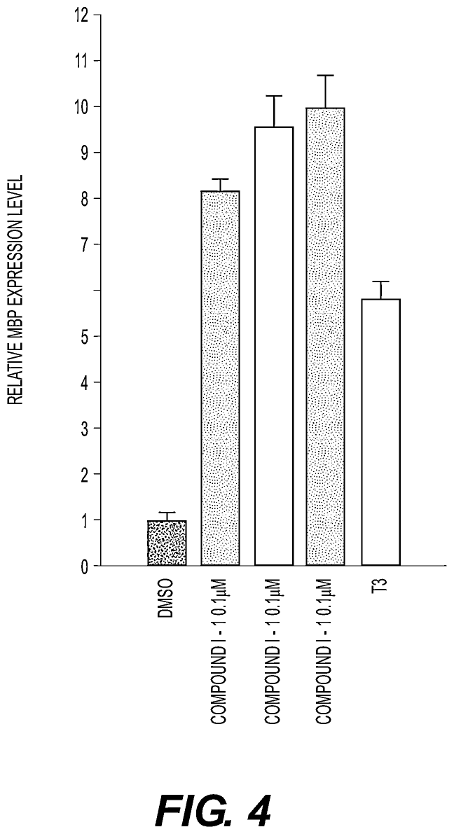

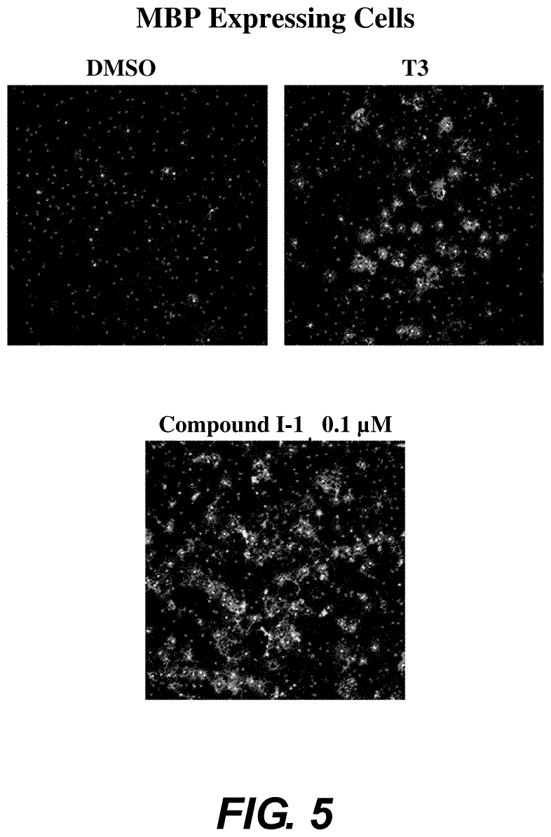

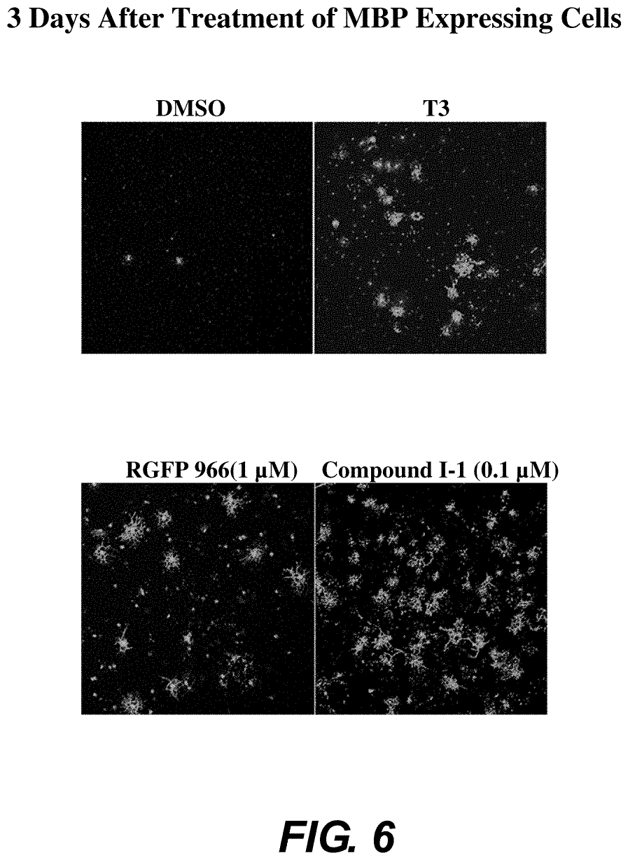

FIGS. 1-6. Primary oligodendrocyte progenitor cells (OPCs) were obtained from newborn rat brains after preparation of mixed glial cultures. This method generally follows that found in ZHAO et al. (2018) “Dual Requirement of CHD8 for Chromatin Landscape Establishment and Histone Methyltransferase Recruitment to Promote CNS Myelination and Repair” Dev Cell, Vol. 45, pp. 753-768. Briefly, mixed glial cells were initially cultured in DMEM-F12 medium supplied with 15% FBS, then switched to B104 conditioned medium for 2 days before isolating OPCs by mechanical detachment in an orbital shaker. Isolated rat OPCs were grown in Sato growth medium supplemented with mitogens 10 ng/ml PDGF-AA and 10 ng/ml bFGF. OPCs were then treated with indicated compounds and immunostained with oligodendrocyte myelination markers MBP and OLIG2, and/or a cell death marker cleaved Caspase 3. T3 ((2S)-2-amino-3-[4-(4-hydroxy-3-iodophenoxy)-3,5-diiodophenyl]propanoic acid; CAS 6893-02-3) was used as a positive control; the T3 concentration used was 15 nm. The results suggest that treatment with compound I-1 promoted oligodendrocyte differentiation and myelination in vitro.

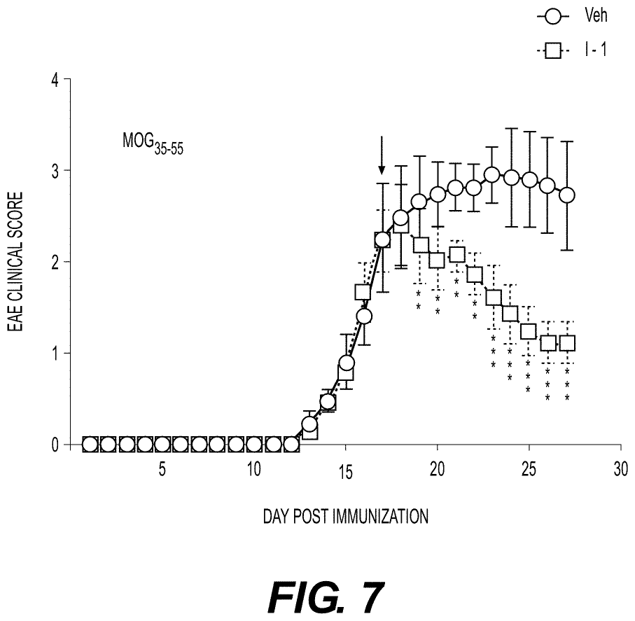

FIG. 7. This method generally follows that found in WANG et al. (2017) “miR-219 Cooperates with miR-338 in Myelination and Promotes Myelin Repair in the CNS” Dev Cell, Vol. 40, pp. 566-582. Briefly, in the experimental autoimmune encephalomyelitis (EAE) demyelinating mouse model induced by myelin peptide MOG35-55 (e.g., see Bittner et al. (2014) “Myelin Oligodendrocyte Glycoprotein (MOG35-55) Induced Experimental Autoimmune Encephalomyelitis (EAE) in C₅₇BL/6 Mice” J Vis Exp. Vol. 86, Article 51275 (5 pages)), compound I-1 administration improved motor function reflected in clinical scores as well as in remyelination.

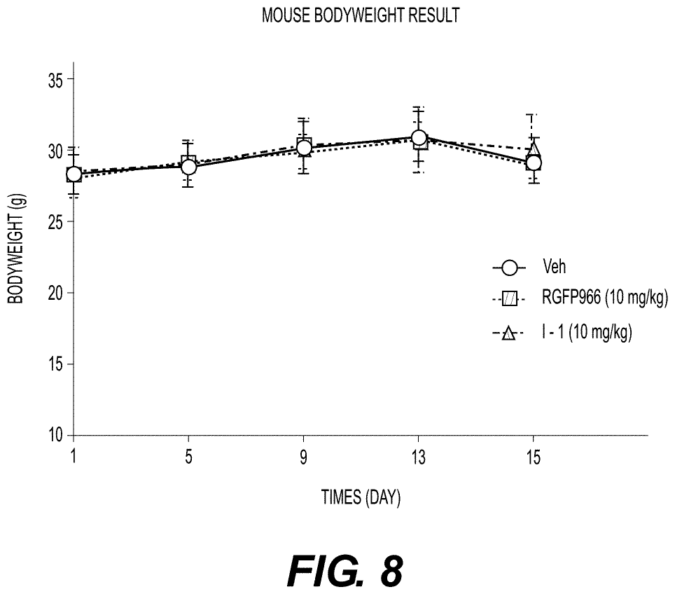

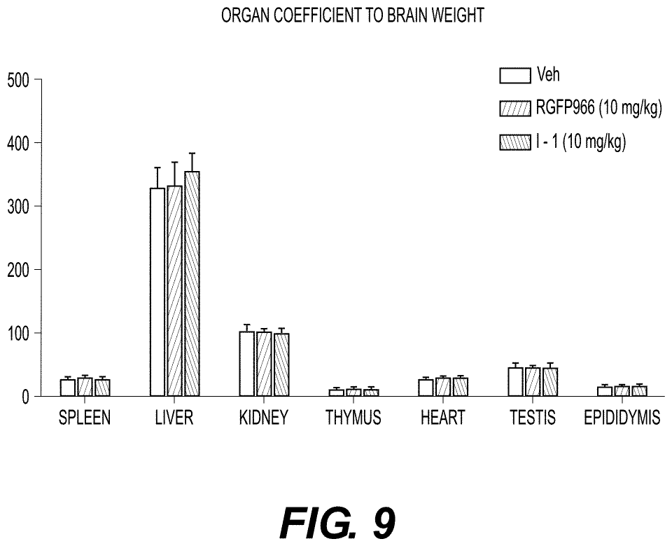

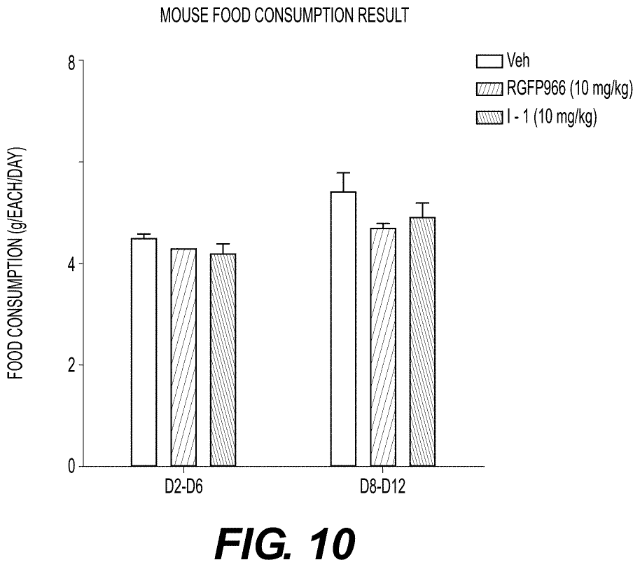

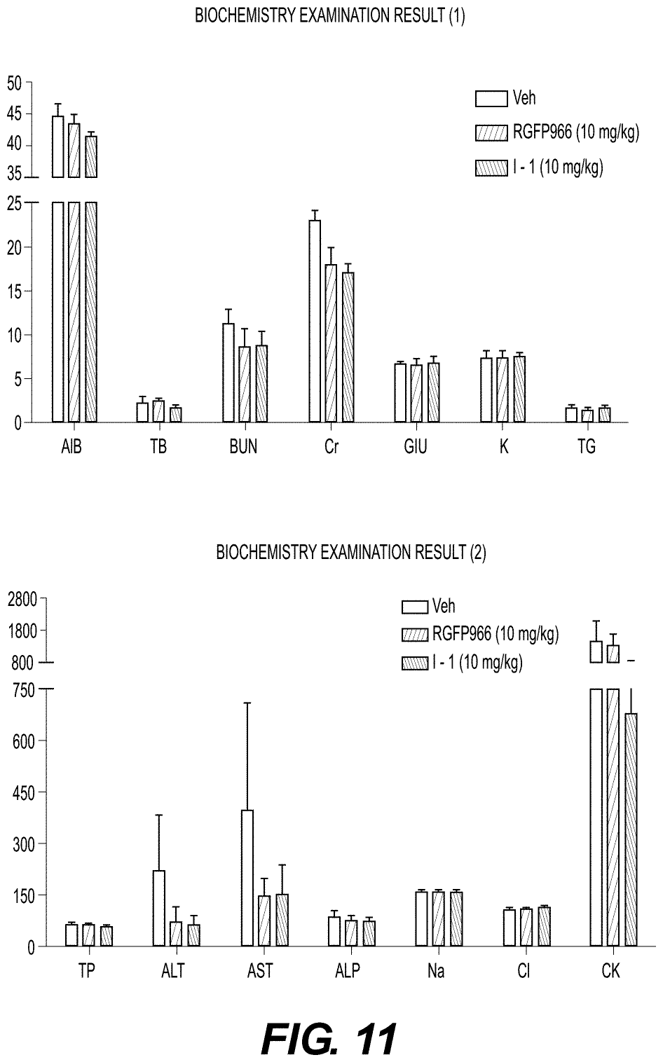

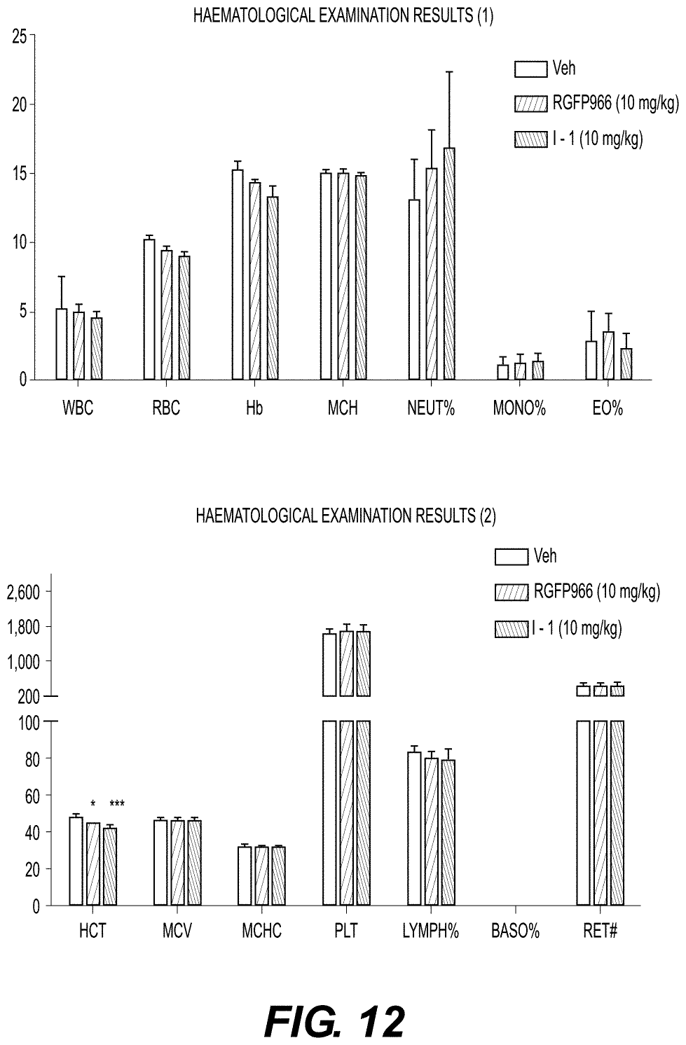

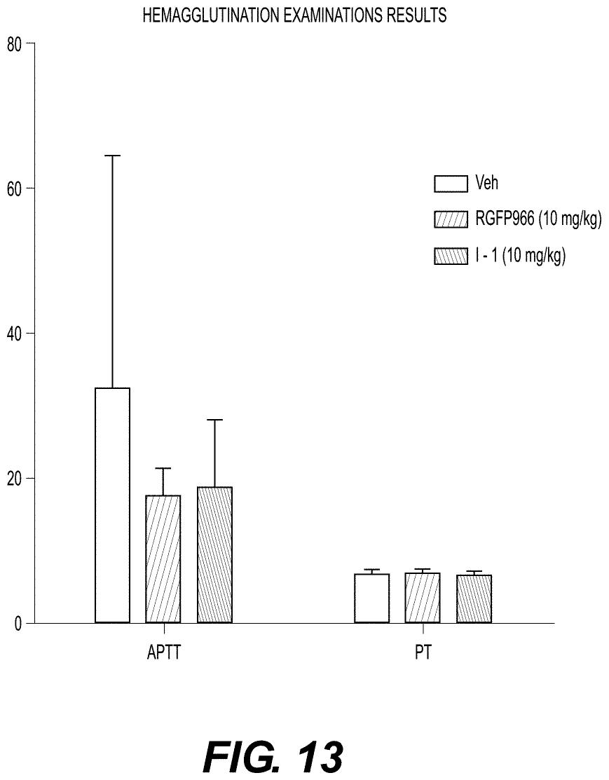

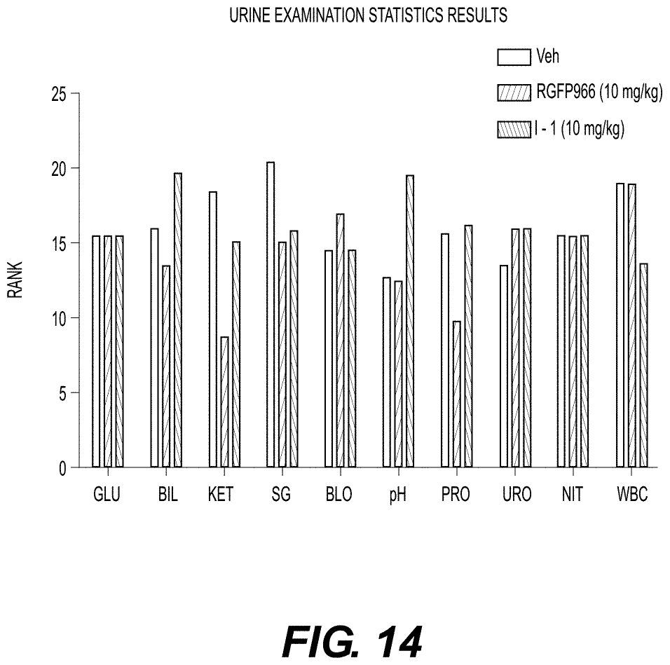

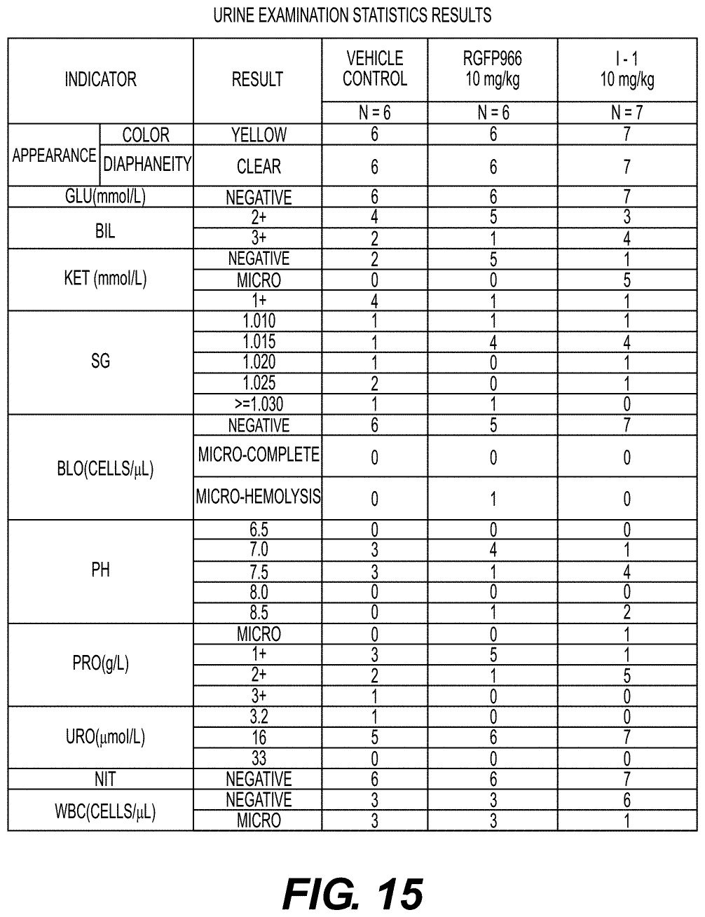

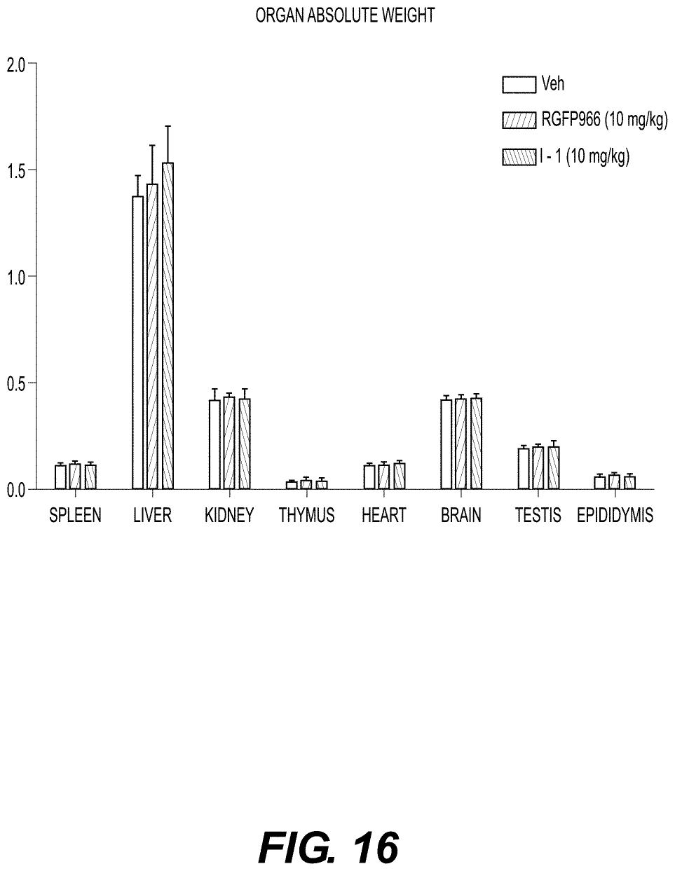

FIGS. 8-16. Adult mice were treated with compound I-1 and RGFP966

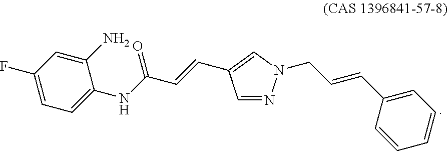 [SMILES: Nc1cc(F)ccc1NC(=O)/C=C/c1cnn(C/C=C/c2ccccc2)c1]

All the serum and urine samples collected were processed for routine biochemical parameters on the same day of the sample collection. The hematology was measured by XT-2000i haematology analyser. The biochemical parameters of plasma and urine were measured using ROCHE cobas c311 automated chemistry analyzer. The results indicate that treatment with the compounds did not exhibit adverse effects on bodyweight; food consumption; biochemical, hematological, hemagglutination indexes in blood; and urine indexes.

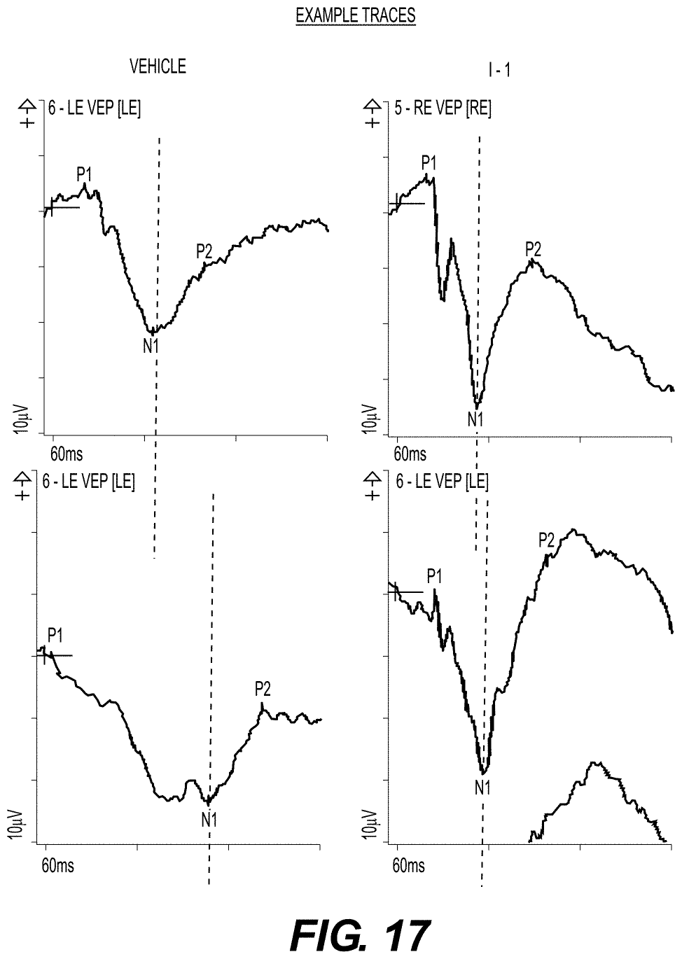

FIG. 17. Visual evoked potential (VEP) analyses were carried out on EAE mice treated with Vehicle and compound I-1 at the peak of disease. This method generally follows that found in YOU et al. (2011) “Latency delay of visual evoked potential is a real measurement of demyelination in a rat model of optic neuritis” Invest Ophthalmol Vis Sci, Vol. 52, pp. 6911-6918. The results indicate that compound I-1 treated animals showed greater and faster conducting VEP N1 signals, suggesting a functional recovery in mice with EAE-induced demyelination.

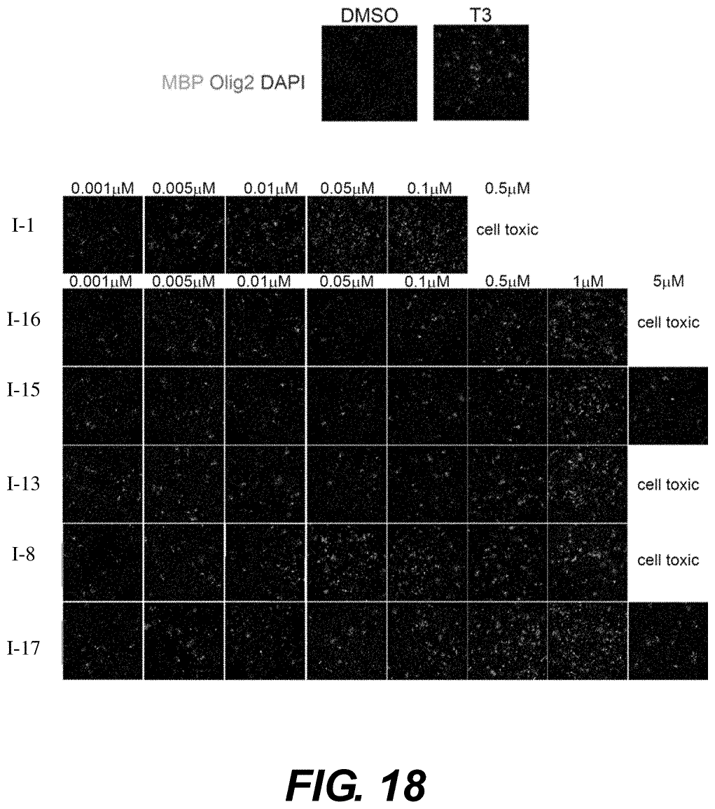

FIG. 18. Methods: This method generally follows that found in WANG et al. (2017) “miR-219 Cooperates with miR-338 in Myelination and Promotes Myelin Repair in the CNS” Dev Cell, Vol. 40, pp. 566-582. Rat oligodendrocyte progenitor cells (OPCs) were seeded on coverslip and treated with compounds, I-1, I-16, I-15, I-13, I-8, and I-17 at the indicated concentration. Cells were immunostained with a mature oligodendrocyte marker MBP and an oligodendrocyte lineage marker Olig2 after 48 h treatment. While all tested compounds showed some activity, compounds I-8 and I-17 show a comparable activity with compound I-1 in promoting OPC into mature oligodendrocytes.

The headings used in the disclosure are not meant to suggest that all disclosure relating to the heading is found within the section that starts with that heading. Disclosure for any subject may be found throughout the specification.

It is noted that terms like “preferably,” “commonly,” and “typically” are not used herein to limit the scope of the claimed invention or to imply that certain features are critical, essential, or even important to the structure or function of the claimed invention. Rather, these terms are merely intended to highlight alternative or additional features that may or may not be utilized in a particular embodiment of the present invention.

As used in the disclosure, “a” or “an” means one or more than one, unless otherwise specified. As used in the claims, when used in conjunction with the word “comprising” the words “a” or “an” means one or more than one, unless otherwise specified. As used in the disclosure or claims, “another” means at least a second or more, unless otherwise specified. As used in the disclosure, the phrases “such as”, “for example”, and “e.g.” mean “for example, but not limited to” in that the list following the term (“such as”, “for example”, or “e.g.”) provides some examples but the list is not necessarily a fully inclusive list. The word “comprising” means that the items following the word “comprising” may include additional unrecited elements or steps; that is, “comprising” does not exclude additional unrecited steps or elements.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

Detailed descriptions of one or more embodiments are provided herein. It is to be understood, however, that the present invention may be embodied in various forms. Therefore, specific details disclosed herein (even if designated as preferred or advantageous) are not to be interpreted as limiting, but rather are to be used as an illustrative basis for the claims and as a representative basis for teaching one skilled in the art to employ the present invention in any appropriate manner. Indeed, various modifications of the invention in addition to those described herein will become apparent to those skilled in the art from the foregoing description and the accompanying figures. Such modifications are intended to fall within the scope of the appended claims.

What is claimed is:

1. A compound selected from Formula (I) 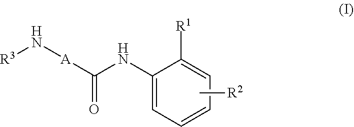
and salts, optical isomers, geometric isomers, salts of isomers, and derivatives thereof;
wherein
R¹ is —NH₂, hydroxy (—OH), —SH, —CN, methanoyl (—COH), or carboxy (—CO₂H);
R² is monovalent H, halogen, hydroxy (—OH), methanoyl (—COH), carboxy (—CO₂H), nitro (—NO₂), —NH₂, —N(CH₃)₂, cyano (—CN), ethynyl (—CCH), propynyl, sulfo (—SO₃H), —CONH₂, —CON(CH₃)₂, C₁-C₃ alkyl, C₁-C₃ perfluorinated alkyl, —CF₃, —OCF₃, or C₁-C₃ alkoxy;
A is a cycloalkyl, heterocyclyl, aryl, or heteroaryl, which cycloalkyl, heterocyclyl, aryl, or heteroaryl is optionally substituted with one or more of halogen, oxo (═O), hydroxy (—OH), methanoyl (—COH), carboxy (—CO₂H), nitro (—NO₂), —NH₂, —N(CH₃)₂, cyano (—CN), ethynyl (—CCH), propynyl, sulfo (—SO₃H), morpholinyl, —CO-morpholin-4-yl, phenyl, —CONH₂, —CON(CH₃)₂, C₁-C₃ alkyl, C₁-C₃ perfluorinated alkyl, —CF₃, —OCF₃, or C₁-C₃ alkoxy;
R³ is methanoyl (—COH), carboxy (—CO₂H), C₁-C₁₀ alkyl, C₂-C₁₀ alkenyl, C₂-C₁₀ alkynyl, C₁-C₉ alkoxy, cycloalkyl, heterocyclyl, aryl, heteroaryl, or R^{Pa}, which methanoyl (—COH), carboxy (—CO₂H), C₁-C₁₀ alkyl, C₂-C₁₀ alkenyl, C₂-C₁₀ alkynyl, C₁-C₉ alkoxy, cycloalkyl, heterocyclyl, aryl, or heteroaryl is optionally substituted with one or more of halogen, oxo (═O), hydroxy (—OH), methanoyl (—COH), carboxy (—CO₂H), nitro (—NO₂), —NH₂, —N(CH₃)₂, cyano (—CN), ethynyl (—CCH), propynyl, sulfo (—SO₃H), morpholinyl, —CO-morpholin-4-yl, —O—CH₂-heteroaryl, —O—CH₂-heterocyclyl, —O—CH₂-phenyl, —O—CH₂-pyridinyl, —O—CH₂-pyrimidinyl, —O—CH₂-pyrazinyl, —CONH₂, —CON(CH₃)₂, C₁-C₅ alkyl, C₁-C₃ alkyl, methyl, ethyl, propyl, C₁-C₃ perfluorinated alkyl, —CF₃, —OCF₃, C₁-C₅ alkoxy, C₁-C₃ alkoxy, —O-phenyl, cycloalkyl, heterocyclyl, aryl, heteroaryl, pyridinyl, pyrimidinyl, pyrazinyl, —O(CO)H, —O(CO)(C₁-C₅ alkyl), —NH(CO)(C₁-C₅ alkyl), —N(C₁-C₅ alkyl)₂, —NH(C₁-C₅ alkyl), —O(CO)(C₁-C₃ alkyl), —NH(CO)(C₁-C₃ alkyl), —N(C₁-C₃ alkyl)₂, —NH(C₁-C₃ alkyl), —O(CO)(phenyl), —NH(CO)(phenyl), —N(C₁-C₃ alkyl)(phenyl), —NH(phenyl), phenyl, or R^{P};
R^{P} is a phenyl substituted with one or more of halogen, hydroxy (—OH), methanoyl (—COH), carboxy (—CO₂H), nitro (—NO₂), —NH₂, —N(CH₃)₂, cyano (—CN), ethynyl (—CCH), propynyl, sulfo (—SO₃H), morpholinyl, —CO-morpholin-4-yl, —O—CH₂-heteroaryl, —O—CH₂-heterocyclyl, —O—CH₂-phenyl, —O—CH₂-pyridinyl, —O—CH₂-pyrimidinyl, —O—CH₂-pyrazinyl, —CONH₂, —CON(CH₃)₂, C₁-C₅ alkyl, C₁-C₃ alkyl, C₁-C₃ perfluorinated alkyl, —CF₃, —OCF₃, C₁-C₅ alkoxy, C₁-C₃ alkoxy, —O-phenyl, methyl, ethyl, propyl, cycloalkyl, heterocyclyl, aryl, heteroaryl, phenyl, pyridinyl, pyrimidinyl, pyrazinyl, —O(CO)H, —O(CO)(C₁-C₅ alkyl), —NH(CO)(C₁-C₅ alkyl), —N(C₁-C₅ alkyl)₂, —NH(C₁-C₅ alkyl), —O(CO)(C₁-C₃ alkyl), —NH(CO)(C₁-C₃ alkyl), —N(C₁-C₃ alkyl)₂, —NH(C₁-C₃ alkyl), —O(CO)(phenyl), —NH(CO)(phenyl), —N(C₁-C₃ alkyl)(phenyl), or —NH(phenyl);
R^{Pa} is a phenyl optionally substituted with one or more of halogen, hydroxy (—OH), methanoyl (—COH), carboxy (—CO₂H), nitro (—NO₂), —NH₂, —N(CH₃)₂, cyano (—CN), ethynyl (—CCH), propynyl, sulfo (—SO₃H), morpholinyl, —CO-morpholin-4-yl, —O—CH₂-heteroaryl, —O—CH₂-heterocyclyl, —O—CH₂-phenyl, —O—CH₂-pyridinyl, —O—CH₂-pyrimidinyl, —O—CH₂-pyrazinyl, —CONH₂, —CON(CH₃)₂, C₁-C₅ alkyl, C₁-C₃ alkyl, C₁-C₃ perfluorinated alkyl, —CF₃, —OCF₃, C₁-C₅ alkoxy, C₁-C₃ alkoxy, —O— phenyl, methyl, ethyl, propyl, cycloalkyl, heterocyclyl, aryl, heteroaryl, phenyl, pyridinyl, pyrimidinyl, pyrazinyl, —O(CO)H, —O(CO)(C₁-C₅ alkyl), —NH(CO)(C₁-C₅ alkyl), —N(C₁-C₅ alkyl)₂, —NH(C₁-C₅ alkyl), —O(CO)(C₁-C₃ alkyl), —NH(CO)(C₁-C₃ alkyl), —N(C₁-C₃ alkyl)₂, —NH(C₁-C₃ alkyl), —O(CO)(phenyl), —NH(CO)(phenyl), —N(C₁-C₃ alkyl)(phenyl), —NH(phenyl), or R^{P}; and
R^{P} and R^{Pa} is the same or different.

2. The compound of claim 1, wherein R¹ is —NH₂, R^{Pa} is a substituted phenyl, or both.

3. The compound of claim 1 or claim 2, wherein R² is (a) (i) meta to R¹ and para to the amide or (ii) para to R¹ and meta to the amide, (b) monovalent H, halogen, hydroxy (—OH), cyano (—CN), methyl, ethyl, or methoxy, or (c) both (a) and (b).

4. The compound of any of claims 1-3, wherein A is a substituted or unsubstituted 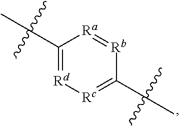
wherein
R^{a}, R^{b}, R^{c}, and R^{d} is CH or N;
R^{a}, R^{b}, R^{c}, and R^{d} is the same or different from each other;
the number of Ns in A is 0, 1, 2, or 3; and
if A is substituted, it is substituted with one or more of halogen, hydroxy (—OH), methanoyl (—COH), carboxy (—CO₂H), nitro (—NO₂), —NH₂, —N(CH₃)₂, cyano (—CN), ethynyl (—CCH), propynyl, sulfo (—SO₃H), morpholinyl, —CO-morpholin-4-yl, phenyl, —CONH₂, —CON(CH₃)₂, C₁-C₃ alkyl, C₁-C₃ perfluorinated alkyl, —CF₃, —OCF₃, or C₁-C₃ alkoxy.

5. The compound of any of claims 1-4, wherein A is

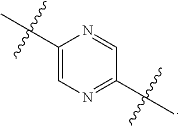 [SMILES: *c1cnc(*)cn1]

6. The compound of any of claims 1-5, wherein R^{P} is 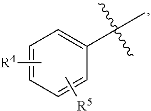
wherein R⁴ and R⁵ is the same or different, is ortho, para, or meta to each other, is each independently ortho, para, or meta to the attachment point, and is H, halogen, hydroxy (—OH), methanoyl (—COH), carboxy (—CO₂H), nitro (—NO₂), —NH₂, —N(CH₃)₂, cyano (—CN), ethynyl (—CCH), propynyl, sulfo (—SO₃H), morpholinyl, —CO-morpholin-4-yl, —O—CH₂-heteroaryl, —O—CH₂-heterocyclyl, —O—CH₂-phenyl, —O—CH₂-pyridinyl, —O—CH₂-pyrimidinyl, —O—CH₂-pyrazinyl, —CONH₂, —CON(CH₃)₂, C₁-C₅ alkyl, C₁-C₃ alkyl, C₁-C₃ perfluorinated alkyl, —CF₃, —OCF₃, C₁-C₅ alkoxy, C₁-C₃ alkoxy, —O-phenyl, methyl, ethyl, propyl, cycloalkyl, heterocyclyl, aryl, heteroaryl, phenyl, pyridinyl, pyrimidinyl, pyrazinyl, —O(CO)H, —O(CO)(C₁-C₅ alkyl), —NH(CO)(C₁-C₅ alkyl), —N(C₁-C₅ alkyl)₂, —NH(C₁-C₅ alkyl), —O(CO)(C₁-C₃ alkyl), —NH(CO)(C₁-C₃ alkyl), —N(C₁-C₃ alkyl)₂, —NH(C₁-C₃ alkyl), —O(CO)(phenyl), —NH(CO)(phenyl), —N(C₁-C₃ alkyl)(phenyl), or —NH(phenyl).

7. The compound of any of claims 1-6, wherein R⁴ and R⁵ is the same or different, is ortho, para, or meta to each other, is each independently ortho, para, or meta to the attachment point, and is H, F, Cl, Br, hydroxy (—OH), cyano (—CN), —CF₃, methyl, ethyl, methoxy, ethoxy, —O(CO)CH₃, or —NH—(CO)—CH₃.

8. The compound of any of claims 1-7, wherein R^{Pa} 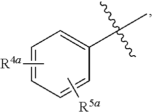
wherein R^{4a} and R^{5a} is the same or different, is ortho, para, or meta to each other, is each independently ortho, para, or meta to the attachment point, and is H, halogen, hydroxy (—OH), methanoyl (—COH), carboxy (—CO₂H), nitro (—NO₂), —NH₂, —N(CH₃)₂, cyano (—CN), ethynyl (—CCH), propynyl, sulfo (—SO₃H), morpholinyl, —CO-morpholin-4-yl, —O—CH₂-heteroaryl, —O—CH₂-heterocyclyl, —O—CH₂-phenyl, —O—CH₂-pyridinyl, —O—CH₂-pyrimidinyl, —O—CH₂-pyrazinyl, —CONH₂, —CON(CH₃)₂, C₁-C₅ alkyl, C₁-C₃ alkyl, C₁-C₃ perfluorinated alkyl, —CF₃, —OCF₃, C₁-C₅ alkoxy, C₁-C₃ alkoxy, —O-phenyl, methyl, ethyl, propyl, cycloalkyl, heterocyclyl, aryl, heteroaryl, phenyl, pyridinyl, pyrimidinyl, pyrazinyl, —O(CO)H, —O(CO)(C₁-C₅ alkyl), —NH(CO)(C₁-C₅ alkyl), —N(C₁-C₅ alkyl)₂, —NH(C₁-C₅ alkyl), —O(CO)(C₁-C₃ alkyl), —NH(CO)(C₁-C₃ alkyl), —N(C₁-C₃ alkyl)₂, —NH(C₁-C₃ alkyl), —O(CO)(phenyl), —NH(CO)(phenyl), —N(C₁-C₃ alkyl)(phenyl), or —NH(phenyl).

9. The compound of any of claims 1-8, wherein R^{4a} and R^{5a} is the same or different, is ortho, para, or meta to each other, is each independently ortho, para, or meta to the attachment point, and is H, F, Cl, Br, hydroxy (—OH), cyano (—CN), —CF₃, methyl, ethyl, methoxy, ethoxy, —O(CO)CH₃, or —NH—(CO)—CH₃.

10. The compound of any of claims 1-9, wherein R³ is 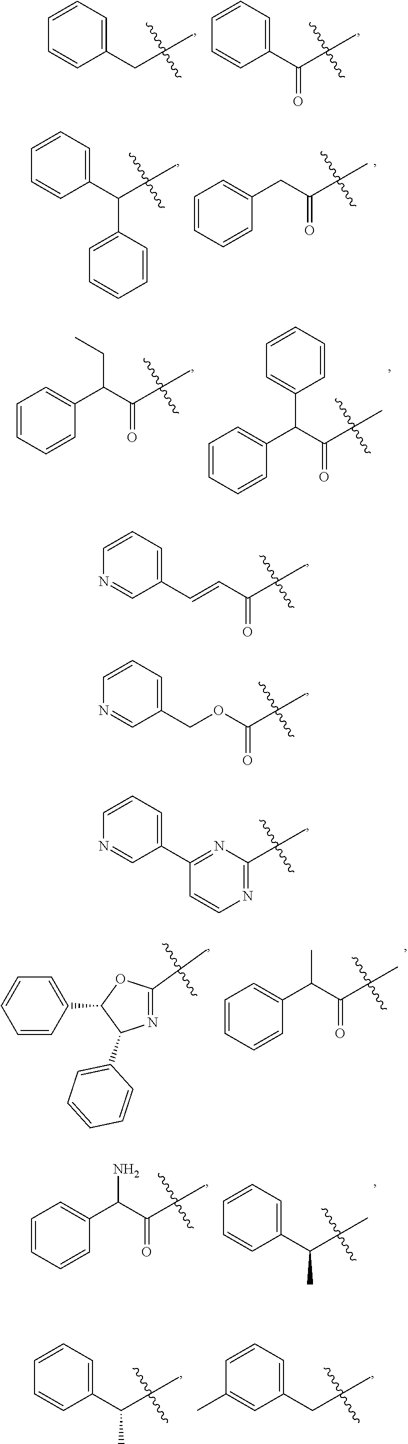 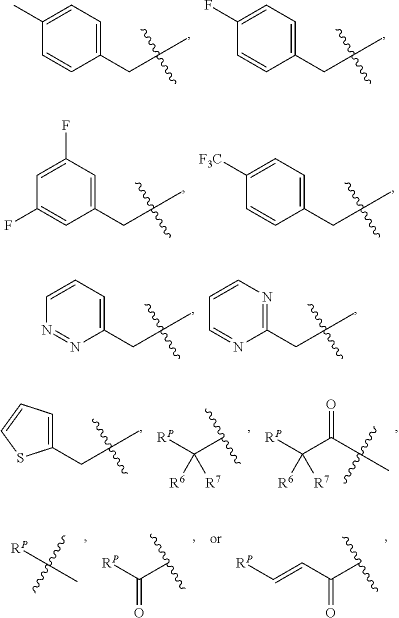
where R⁶ and R⁷ is the same or different and is H, methyl, ethyl, phenyl, R^{P}, or pyridinyl.

11. The compound of any of claims 1-10, wherein R³ is 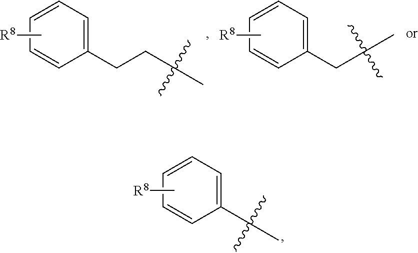
where R⁸ is ortho, para or meta to the attachment point or to the carbon in the phenyl associated with the attachment point, and is H, halogen (e.g., F, Cl, Br, or I), hydroxy (—OH), —CF₃, —CF₂CF₃, methanoyl (—COH), carboxy (—CO₂H), nitro (—NO₂), —NH₂, —N(CH₃)₂, cyano (—CN), sulfo (—SO₃H), —CONH₂, —CON(CH₃)₂, C₁-C₅ alkyl, C₁-C₃ alkyl, C₁-C₅ alkoxy, C₁-C₃ alkoxy, —O— phenyl, methyl, ethyl, propyl, phenyl, pyridinyl, pyrimidinyl, pyrazinyl, pyridazinyl, thienyl, —O(CO)H, —O(CO)(C₁-C₅ alkyl), —NH(CO)(C₁-C₅ alkyl), —N(C₁-C₅ alkyl)₂, —NH(C₁-C₅ alkyl), —O(CO)(C₁-C₃ alkyl), —NH(CO)(C₁-C₃ alkyl), —N(C₁-C₃ alkyl)₂, —NH(C₁-C₃ alkyl), —O(CO)(phenyl), —NH(CO)(phenyl), —N(C₁-C₃ alkyl)(phenyl), or —NH(phenyl).

12. The compound of any of claims 1-11, wherein R³ does not comprise an oxo adjacent to the attachment point.

13. The compound of any of claims 1-12, wherein the compound is selected from Formula (Ia) 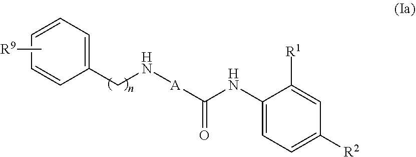
and salts, optical isomers, geometric isomers, salts of isomers, and derivatives thereof;
wherein
n is 0, 1, 2, or 3; and
R⁹ is ortho, para, or meta to the other connecting carbon on the phenyl, and is H, halogen, hydroxy (—OH), methanoyl (—COH), carboxy (—CO₂H), nitro (—NO₂), —NH₂, —N(CH₃)₂, cyano (—CN), ethynyl (—CCH), propynyl, sulfo (—SO₃H), morpholinyl, —CO-morpholin-4-yl, —O—CH₂-heteroaryl, —O—CH₂-heterocyclyl, —O—CH₂-phenyl, —O—CH₂-pyridinyl, —O—CH₂-pyrimidinyl, —O—CH₂-pyrazinyl, —CONH₂, —CON(CH₃)₂, C₁-C₅ alkyl, C₁-C₃ alkyl, C₁-C₃ perfluorinated alkyl, —CF₃, —OCF₃, C₁-C₅ alkoxy, C₁-C₃ alkoxy, —O-phenyl, methyl, ethyl, propyl, cycloalkyl, heterocyclyl, aryl, heteroaryl, phenyl, pyridinyl, pyrimidinyl, pyrazinyl, —O(CO)H, —O(CO)(C₁-C₅ alkyl), —NH(CO)(C₁-C₅ alkyl), —N(C₁-C₅ alkyl)₂, —NH(C₁-C₅ alkyl), —O(CO)(C₁-C₃ alkyl), —NH(CO)(C₁-C₃ alkyl), —N(C₁-C₃ alkyl)₂, —NH(C₁-C₃ alkyl), —O(CO)(phenyl), —NH(CO)(phenyl), —N(C₁-C₃ alkyl)(phenyl), or —NH(phenyl).

14. The compound of claim 13, wherein R⁹ is ortho, para, or meta to the other connecting carbon on the phenyl, and is H, F, Cl, Br, hydroxy (—OH), cyano (—CN), methyl, ethyl, methoxy, ethoxy, —CF₃, —O(CO)CH₃, or —NH—(CO)—CH₃.

15. The compound of any of claims 1-14, wherein the compound of Formula (I) is I-1, I-2, I-3, I-4, I-5, I-6, I-7, I-8, I-9, I-10, I-11, I-12, I-13, I-14, I-15, I-16, I-17, I-18, I-19, I-20, I-21, I-22, I-23, I-24, I-25, I-26, or I-27.

16. A composition comprising a compound of any of claims 1-15.

17. The composition of claim 16, wherein the amount of the compound is from about 0.0001% (by weight total composition) to about 99%.

18. The composition of claim 16 or claim 17, further comprising a formulary ingredient, an adjuvant, or a carrier.

19. A pharmaceutical composition comprising a compound of any of claims 1-15.

20. The pharmaceutical composition of claim 19, wherein the amount of the compound is from about 0.0001% (by weight total composition) to about 50%.

21. The pharmaceutical composition of claim 19 or claim 20, further comprising a formulary ingredient, an adjuvant, or a carrier.

22. A method for providing an animal with a compound comprising one or more administrations of one or more compositions comprising the compound of any of claims 1-15, wherein the compositions may be the same or different if there is more than one administration.

23. The method of claim 22, wherein at least one of the one or more compositions further comprises a formulary ingredient.

24. The method of claim 22 or claim 23, wherein at least one of the one or more compositions comprises the composition of any of claims 16-18 or the pharmaceutical composition of any of claims 19-21.

25. The method of any of claims 22-24, wherein at least one of the one or more administrations comprises parenteral administration, a mucosal administration, intravenous administration, subcutaneous administration, topical administration, intradermal administration, oral administration, sublingual administration, intranasal administration, or intramuscular administration.

26. The method of any of claims 22-25, wherein if there is more than one administration at least one composition used for at least one administration is different from the composition of at least one other administration.

27. The method of any of claims 22-26, wherein the compound of at least one of the one or more compositions is administered to the animal in an amount of from about 0.01 mg/kg animal body weight to about 15 mg/kg animal body weight.

28. The method of any of claims 22-27, wherein the animal is a human, a rodent, or a primate.

29. A method for treating an animal for a disease or a nerve injury, comprising one or more administrations of one or more compositions comprising the compound of any of claims 1-15, wherein the compositions may be the same or different if there is more than one administration.

30. The method of claim 29, wherein the composition further comprises a formulary ingredient.

31. The method of claim 29 or claim 30, wherein at least one of the one or more compositions comprises the composition of any of claims 16-18 or the pharmaceutical composition of any of claims 19-21.

32. The method of any of claims 29-31, wherein the composition is a pharmaceutical composition.

33. The method of any of claims 29-32, wherein the administration comprises parenteral administration, mucosal administration, intravenous administration, depot injection, subcutaneous administration, topical administration, intradermal administration, oral administration, sublingual administration, intranasal administration, or intramuscular administration.

34. The method of any of claims 29-33, wherein the administration comprises a depot injection or an oral administration.

35. The method of any of claims 29-34, wherein if there is more than one administration at least one composition used for at least one administration is different from the composition of at least one other administration.

36. The method of any of claims 29-35, wherein the amount of the compound is from about 0.0001% (by weight total composition) to about 99%.

37. The method of any of claims 29-36, wherein the compound is administered to the animal in an amount of from about 0.005 mg/kg animal body weight to about 100 mg/kg animal body weight.

38. The method of any of claims 29-37, wherein the animal is a human, a rodent, or a primate.

39. The method of any of claims 29-38, wherein the animal is in need of treatment of a disease or a nerve injury.

40. The method of any of claims 29-39, wherein the method is for treating myelopathy, spinal cord injury, myelitis, vascular myelopathy, cervical spondylotic myelopathy, spondylosis, spinal stenosis, demyelinating disease, any disease of the nervous system where the myelin sheath of a neuron is damaged, CNS demyelinating disease, PNS demyelinating disease, genetic demyelinating disease, infectious demyelinating disease, autoimmune demyelinating disease, demyelinating myelinoclastic disease, demyelinating leukodystrophic disease, Devic's disease, CNS neuropathies, diseases resulting in vitamin B12 deficiency, central pontine myelinolysis, myelopathies, tabes dorsalis, leukoencephalopathies, progressive multifocal leukoencephalopathy, leukodystrophies, optic neuritis, transverse myelitis, neuromyelitis optica, Guillain-Barré syndrome, chronic inflammatory demyelinating polyneuropathy, anti-MAG peripheral neuropathy, Charcot-Marie-Tooth disease, Hereditary neuropathy with liability to pressure palsy, copper deficiency associated conditions, peripheral neuropathy, myelopathy, optic neuropathy, progressive inflammatory neuropathy, multiple sclerosis (MS), MS-type clinically isolated syndrome, relapsing-remitting MS, primary progressive MS, secondary progressive MS, Alzheimer's Disease, amyotrophic lateral sclerosis (ALS), and Huntington's Disease, traumatic brain injury, acquired brain injury, hypoxic ischemic brain injury, strokes, periventricular leukomalacia (PVL), white-matter brain injury, CNS nerve injury, PNS nerve injury, crush nerve injury, or transection nerve injury.

41. The method of any of claims 29-40, wherein the method is for treating MS, MS-type clinically isolated syndrome, relapsing-remitting MS, primary progressive MS, or secondary progressive MS.

42. The method of any of claims 29-41, wherein the method is for treating inflammation, remyelination, or both in MS, MS-type clinically isolated syndrome, relapsing-remitting MS, primary progressive MS, or secondary progressive MS.

43. The method of any of claims 29-42, wherein the method is for treating inflammation and remyelination in MS, MS-type clinically isolated syndrome, relapsing-remitting MS, primary progressive MS, or secondary progressive MS.

44. The method of any of claims 29-43, wherein the method is for treating CNS demyelinating disease, PNS demyelinating disease, MS, Alzheimer's Disease, amyotrophic lateral sclerosis (ALS), and Huntington's Disease, traumatic brain injury, acquired brain injury, hypoxic ischemic brain injury, strokes, periventricular leukomalacia (PVL), white-matter brain injury, CNS nerve injury, PNS nerve injury, crush nerve injury, or transection nerve injury.

45. The method of any of claims 29-44, wherein the method is for treating CNS demyelinating disease, PNS demyelinating disease, MS, Alzheimer's Disease, amyotrophic lateral sclerosis (ALS), and Huntington's Disease, CNS nerve injury, PNS nerve injury, crush nerve injury, or transection nerve injury.

46. The method of any of claims 29-45, wherein the method is for treating CNS nerve injury, PNS nerve injury, crush nerve injury, or transection nerve injury.

47. The method of any of claims 29-46, wherein the method further comprises one or more other treatments.

48. A method for treating an animal for MS or nerve injury, comprising administration to the animal of a composition comprising a compound selected from Formula (Ia) and salts, optical isomers, geometric isomers, salts of isomers, and derivatives thereof.

49. A method for treating an animal for MS or nerve injury, comprising administration to the animal of a composition comprising a compound selected from compound I-1 and salts, optical isomers, geometric isomers, salts of isomers, and derivatives thereof.

50. A method for preparing a compound of any of claims 1-15 comprising, (a) reacting a compound of Formula (II) with a compound of Formula (III) to result in a mixture comprising a compound of Formula (IV),
(b) reacting a compound of Formula (IV) to result in a mixture comprising a compound of Formula (V),
(c) reacting a compound of Formula (V) with a compound of Formula (VI), and
(d) recovering a compound of Formula (I);
wherein Formula (II) is 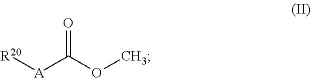
Formula (III) is 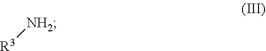
Formula (IV) is 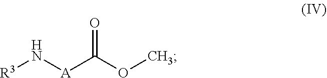
Formula (V) is 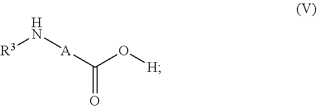
Formula (VI) is 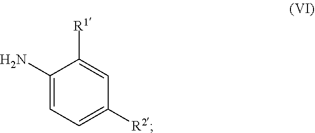
R²⁰ is a halogen;
R^{1′} is R¹ or R¹ with a protecting group; and
R^{2′} is R² or R² with a protecting group.

51. The method of claim 50, wherein the compound is selected from Formula (Ia).

52. The method of claim 50 or claim 51, wherein in step (b) Formula (IV) is reacted with a base.

53. The method of any of claims 50-52, wherein in step (b) Formula (IV) is reacted with a base, and the base is LiOH.

54. The method of any of claims 50-53, wherein one or both of R^{1′} or R^{2′} is a protected R¹ or a protected R².

55. The method of any of claims 50-54, wherein (i) one or both of R^{1′} or R^{2′} is a protected R¹ or a protected R² and (ii) one or both protecting groups are a carbamate, t-butoxycarbonyl (Boc), benzyloxycarbonyl (Cbz), or 9-fluorenylmethoxycarbonyl (Fmoc).

56. The method of any of claims 50-55, wherein (i) one or both of R^{1′} or R^{2′} is a protected R¹ or a protected R² and (ii) after (c), the protecting group(s) are removed from one or both of R^{1′} or R^{2′.}
